(19) United States
(12) Patent Application Publication
(10) Pub. No.: US 2003/0228675 A1
(43) Pub. Date: Dec. 11, 2003

(54) ATM related kinase ATX, nucleic acids encoding same and methods of use

(76) Inventors: Robert T. Abraham, San Diego, CA (US); Diane M. Otterness, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/165,216

(22) Filed: Jun. 6, 2002

(51) Int. Cl. C12N 9/12; C12N 9/22; C07H 21/04; A61K 31/522; C12P 21/02; C12N 5/06

(52) U.S. Cl. 435/199; 435/69.1; 435/320.1; 435/325; 536/23.2; 514/263.3

(57) ABSTRACT

The invention provides an isolated nucleic acid molecule having substantially the same nucleotide sequence as SEQ ID NO:1. Also provided is an isolated oligonucleotide having at least 15 contiguous nucleotides of a nucleotide sequence referenced as SEQ ID NO:11. An isolated polypeptide having substantially the same amino acid sequence as SEQ ID NO:2 is further provided as well as an antibody, or antigen binding fragment thereof, which specifically binds to an ATX polypeptide and has an amino acid sequence as referenced in SEQ ID NO:2. A method for identifying an ATX-modulatory compound is additionally provided. The method consists of measuring the level of an ATX polypeptide in the presence of a test compound, wherein a difference in the level of said ATX polypeptide in the presence of said test compound compared to in the absence of said test compound indicating that said test compound is an ATX-modulatory compound, and wherein said ATX-modulatory compound is not caffeine or wortmannin.

This invention was made with government support under grant number CA76193 awarded by the National Institutes of Health. The United States Government has certain rights in this invention.

### BACKGROUND OF THE INVENTION

This invention relates generally to the fields of molecular biology and molecular medicine and more specifically to the identification of phosphoinositide 3-kinase related kinases (PIKKS) involved in cell cycle control and mRNA surveillance pathways.

The mitotic cell cycle is the process by which a cell creates an exact copy of its chromosomes and then segregates each copy into two cells. The sequence of events of the cell cycle is regulated such that cell division does not occur until the cell has completed accurate DNA replication. To ensure that cells pass accurate copies of their genomes on to the next generation, evolution has overlaid the core cell cycle machinery with a series of surveillance pathways termed cell cycle checkpoints. The overall function of these checkpoints is to detect damaged or abnormally structured DNA, and to coordinate cell cycle progression with DNA repair.

Members of the phosphoinositide 3-kinase related kinases (PIKK) family of kinases are involved in cell cycle checkpoints and DNA damage repair. The PIKK family members identified to date express a carboxylterminal domain that displays significant sequence homology to the catalytic domains of phosphoinositide (PI) 3-kinases. Indeed, many, but not all of the PIKKs have been shown to possess protein serine-threonine kinase activities (McMahon et al., Cell 94:363-374 (1998); Vassilev et al., Cell 2:869-875 (1998); Grant et al., Cell 2:863-867 (1998); Hunter, Cell 83:1-4 (1995); Tibbetts and Abraham, Signaling Networks and Cell Cycle: Themolecular Basis of Cancer and Other Diseases pp. 267-301 (2000)). In mammalian cells, three PIKK family members, ATM, ATR, and DNA-dependent protein kinase (DNA-PK), serve as proximal signal transducers in cell-cycle checkpoint and DNA repair pathways (Abraham, Genes & Dev. 15:2177-2196 (2001); Durocher and Jackson, Curr. Opin. Cell Biol. 13:2225-231 (2001)). The critical roles of ATM in orchestrating cellular responses to various forms of stress are underscored by the diverse pathologies associated with the hereditary disorder, ataxiatelangiectasia (A-T) (Crawford, Seminarsin Ped. Neuro. 5:287-294 (1998); Rotman and Shiloh, Human Mol. Gen. 7:1555-1563 (1998); Rotman and Shiloh, Oncogene 18:6135-6144 (1999)). A-T patients lack functional ATM and develop symptoms including extreme sensitivity to irradiation, cerebellar degeneration, oculocutaneous telangiectasias, gonadal deficiencies, immunodeficiencies, and increased risk of cancer (Lehman and Carr, Trends in Genet. 11:375-377 (1995)). Fibroblasts derived from these patients show defects in cell cycle checkpoints and are defective in their response to irradiation (Painter and Young, Proc. Natl. Acad. Sci. (USA) 77:7315-7317 (1980)).

In general, the proteins in the PIKK family of kinases play important roles in mRNA surveillance and cell cycle progression in order to insure genetic integrity from generation to generation. Compounds that modulate PIKK polypeptides can result in altered progression through the cell cycle leading to increased or decreased cell survival. For example, a PIKK-modulatory compound can make a cell more or less susceptible to cell death in the presence of radiation or a cytotoxic agent.

All cancer cells have a dysfunctional cell cycle and continue through the cell cycle in an inappropriate manner, either by failing to respond to negative growth signals or by failing to die in response to the appropriate signal. In addition, most cancer cells lack genomic integrity and often have an increased chromosome count compared to normal cells. Therefore, compounds that inhibit cell cycle checkpoints or DNA damage repair, in combination with the cytotoxic agents, can cause cancer cell death by forcing cancer cells to progress through the cell cycle in the presence of DNA damaging agents such that they undergo events that lead to cell death.

Thus, there exists a need to identify additional members of the PIKK family of kinases and compounds that modulate these kinases. The present invention satisfies this need and provides related advantages as well.

### SUMMARY OF THE INVENTION

The invention provides an isolated nucleic acid molecule having substantially the same nucleotide sequence as SEQ ID NO:1. Also provided is an isolated oligonucleotide having at least 15 contiguous nucleotides of a nucleotide sequence referenced as SEQ ID NO:11. An isolated polypeptide having substantially the same amino acid sequence as SEQ ID NO:2 is further provided as well as an antibody, or antigen binding fragment thereof, which specifically binds to an ATX polypeptide and has an amino acid sequence as referenced in SEQ ID NO:2. A method for identifying an ATX-modulatory compound is additionally provided. The method consists of measuring the level of an ATX polypeptide in the presence of a test compound, wherein a difference in the level of said ATX polypeptide in the presence of said test compound compared to in the absence of said test compound indicating that said test compound is an ATX-modulatory compound, and wherein said ATX-modulatory compound is not caffeine or wortmannin.

### BRIEF DESCRIPTION OF THE DRAWINGS

FIG. 1A shows the genomic structure of the ATX locus along with clones isolated to date. The black diamonds denote the locations of translational stop codons and black bars indicate open reading frames that give rise to various ATX polypeptides. The lines and symbols below exon 15 indicate an allelic variant that contains a 27 bp insertion having two in-frame stop codons. FIG. 1B shows the location of N-terminal homology 1 (NH1), NH2, PI3-K catalytic (PI3-Kc), PKC-λ/L-interacting protein (LIP), and FAT-C (C) domains. The numbers shown indicate identity/similarity and shading highlights amino acid identity with ATX. A sequence alignment of the PI 3-Kc domains of ATX, CeSMG-1, mTOR, and ATM is shown. FIG. 1C shows immune complex kinase assays with GST-p53₁₋₇₀, GST-p53₁₋₇₀ (S15A), or with GSThUpf11019-1118 as substrates. The reaction products were immunoblotted with α-HA (lower panel). FIG. 1D shows immune complex assays with cells or α-HA-ATX immunoprecipitates treated with wortmannin.

FIG. 2A shows clonogenic survival assays after UV exposure. FIG. 2B shows clonogenic survival assays after IR exposure. The upper panel displays colony outgrowth results from cells transfected with the indicated plasmids, and not exposed to IR. FIG. 2C shows clonogenic survival assays of cells treated with ATX-directed antisense oligonucleotides (AS). The right panel displays colony survival results from non-irradiated cells treated with the indicated oligonucleotides.

FIG. 3A shows whole cell extracts resolved by SDS-PAGE and sequentially immunblotted with the indicated antibodies. FIG. 3B shows extracts of transfected cells separated by SDS-PAGE and sequentially immunoblotted with the indicated antibodies. The p53 phosphoserine-15 specific antibody is designated α-pSer15. FIG. 3C shows extracts of cells treated with S or AS oligonucleotides and analyzed as described in panel A. FIG. 3D shows cell cycle progression in AS-transfected cells examined by flow cytometry. The table shows the percentages of cells in each cell cycle phase. The right panel shows immunoblot analyses from the same cell population. FIG. 3E shows an effect of caffeine on AS-induced cell cycle defects. The table shows percentages of cells in each cell-cycle phase, plus the ratio of G2/M to G1 cells for each sample. The right panel shows immunoblotting results from the same cell populations.

FIG. 4A shows an effect of ATX overexpression on basal viability. Cell densities of the scanned images from each sample were obtained with the ImagePro Plus software program. FIG. 4B shows an effect of ATX overexpression on radiosensitivity. Surviving cells were quantitated as described in panel except that arbitrary unit values for each group were normalized to the corresponding nonirradiated control.

FIG. 5A shows cells transfected with GAL4 or GAL4-hUpf11019-1118 expression constructs with the indicated samples treated with wortmannin. The right panel shows phosphatase treatment. The soluble proteins were separated by SDS-PAGE and immunoblotted with α-GAL4 mAb. The arrow indicates the uppermost band of the phosphorylated GAL4-hUpf11019-1118 reporter protein. FIG. 5B shows an effect of ATMKI or ATXKI expression on UV stimulation of GAL4-hUpf1 phosphorylation.

FIG. 6A shows an effect of HA-ATXKI or HAATMKI expression on NMD. Nuclear RNA was isolated from transfected cells and β-globin and MUP mRNAs were quantitated by RT-PCR and PhosphorImaging. For each pair of transfections, the level of Globin mRNA was normalized to the level of MUP mRNA and expressed below each lane as a percentage of the normalized level of Globin Norm mRNA, which was defined as 100. FIG. 6B shows an effect of ATX AS oligonucleotide on NMD.

### DETAILED DESCRIPTION OF THE INVENTION

This invention is directed to isolated ATX nucleic acids and polypeptides. ATX is a novel PIKK kinase family member that participates in stress-induced p53 and cell cycle checkpoint activation in cells exposed to DNA damaging agents. In addition, ATX can activate the intrinsic non-sense mediated mRNA decay (NMD) pathway in these cells. The invention is also directed to methods of identifying ATX-modulatory compounds and using these compounds to modulate cell survival. Compounds that modulate cellular survival can be useful in the treatment of diseases characterized by excessive cell growth or excessive cell death.

In one embodiment, an expressed sequence tag (EST) with homology to a conserved region in the catalytic domains of PIKK family members was used to isolate a full-length cDNA encoding a novel member of the PIKK family, termed ATX. The ATX polypeptide was detected in both the nucleus and cytoplasm of human cells, and formed nuclear foci upon exposure to UV light. In addition, the cell cycle regulatory proteins p53 and hUpf1 were found to be phosphorylated by ATX. Furthermore, the reduction of endogenous ATX in a cell using anti-sense oligonucleotides resulted in decreased survival of cells, and decreased phosphorylation and stabilization of p53 in cells exposed to UV light. Similar to other PIKK family members, ATX activity was inhibited by wortmannin and caffeine.

As used herein, the term “ATX polypeptide” refers to a polypeptide with substantially the same amino acid sequence as that shown in SEQ ID NO:2 (human ATX). “Substantially the same amino acid sequence” is intended to mean an amino acid sequence contains a considerable degree of sequence identity or similarity, such as at least 70%, 80%, 90%, 95%, 98%, or 100% sequence identity or similarity, to a reference amino acid sequence. Substantially the same amino acid sequence includes conservative and non-conservative amino acid changes, gaps, and insertions to an amino acid sequence. Conservative and non-conservative amino acid changes, gaps, and insertions to an amino acid sequence can be compared to a reference sequence using available algorithms and programs such as the Smith-Waterman algorithm and the BLAST homology search program (Altschul et al., J. Mol. Biol. 215:403-410 (1990)).

It is understood that a fragment of ATX can be sufficient in order to produce an ATX activity. Activities associated with ATX include, for example, kinase activity, cell cycle checkpoint activity, and NMD activity. For example, fragments of ATX which retain substantially an activity of the entire polypeptide are included within the definition. Fragments can include, for example, amino terminal, carboxyl terminal, or internal deletions of a full length ATX polypeptide. In addition, fragments can include domains of a full length ATX polypeptide, such as for example, a kinase domain, NH1 domain, NH2 domain, or LIP domain. A fragment can contain, for example, at least about 10, 100, 500, 1,000, 1,500, 2,000, 2,500, 3,000, 3,500 or more contiguous or non-contiguous amino acid residues of a full-length ATX polypeptide. ATX polypeptide fragments include the fragments described above, but excludes fragments KIAA0421 (Accession number AB007881), KIAA0220 (Accession number D86974), and LIP (Accession number U32581), which are present in databases. Polypeptide fragments can be generated using a variety of methods. For example, polypeptide fragments can be generated using recombinant DNA methods, enzymatic cleavage, or chemical cleavage of larger polypeptides.

It is understood that limited modifications to the ATX polypeptide can be made without destroying an activity of ATX. For example, ATX is intended to include other ATX family members such as those polypeptides that are found to exhibit the above sequence homologies. Such members include, for example, homologs of ATX that can be cloned from other organisms such as monkeys, cows, rats, mice, chickens, frogs, flies or worms. The sequence of possible homologs of human ATX are available in nucleotide databases.

Various modifications of the ATX primary amino acid sequence can result in polypeptides having substantially equivalent, decreased, or enhanced function as compared to the sequence set forth as SEQ ID NO:2. Those skilled in the art recognize that such modifications can be desirable at times in order to enhance the bioactivity, bioavailability or stability of ATX, or to facilitate its synthesis or purification. Contemplated amino acid substitutions to the native sequence of ATX can include, for example, conservative changes, wherein a substituted amino acid has similar structural or chemical properties such as replacement of a polar amino acid with another polar amino acid or replacement of a charged amino acid with a similarly charged amino acid. Those skilled in the art also recognize that nonconservative changes such as replacement of an uncharged polar amino acid with an non-polar amino acid or replacement of a charged amino acid with an uncharged polar amino acid, can also be made without affecting a function of ATX. In addition, a variety of polypeptide modifications are known in the art for constraining the structure of polypeptides to enhance stability or binding (Cabezas and Satterthwait, J. Am. Chem. Soc. 121:3862-3875 (1999); Stanfield et al., Structure 7:131-142 (1999)).

A polypeptide can be modified by naturally occurring modifications such as post-translational modifications, including phosphorylation, lipidation, prenylation, sulfation, hydroxylation, acetylation, addition of carbohydrate, addition of prosthetic groups or cofactors, formation of disulfide bonds, proteolysis, assembly into macromolecular complexes, and the like. Chemical modifications of the polypeptide such as, for example, alkylation, acylation, carbamylation, and iodination can also be used to modify an ATX polypeptide. In addition, various molecules, such as other polypeptides, carbohydrates, or lipids, or small molecules can be attached to ATX including fragments of ATX. For example, ATX can contain a label moiety, a sequence such as a FLAG epitope, or be fused to another polypeptide such as a DNA binding domain.

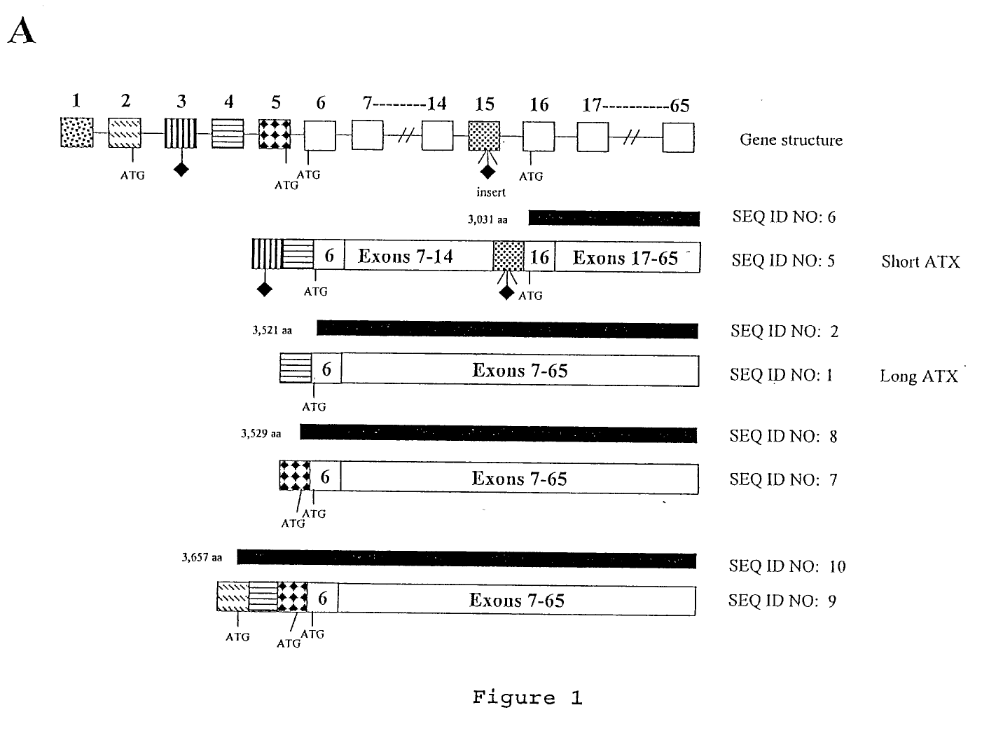

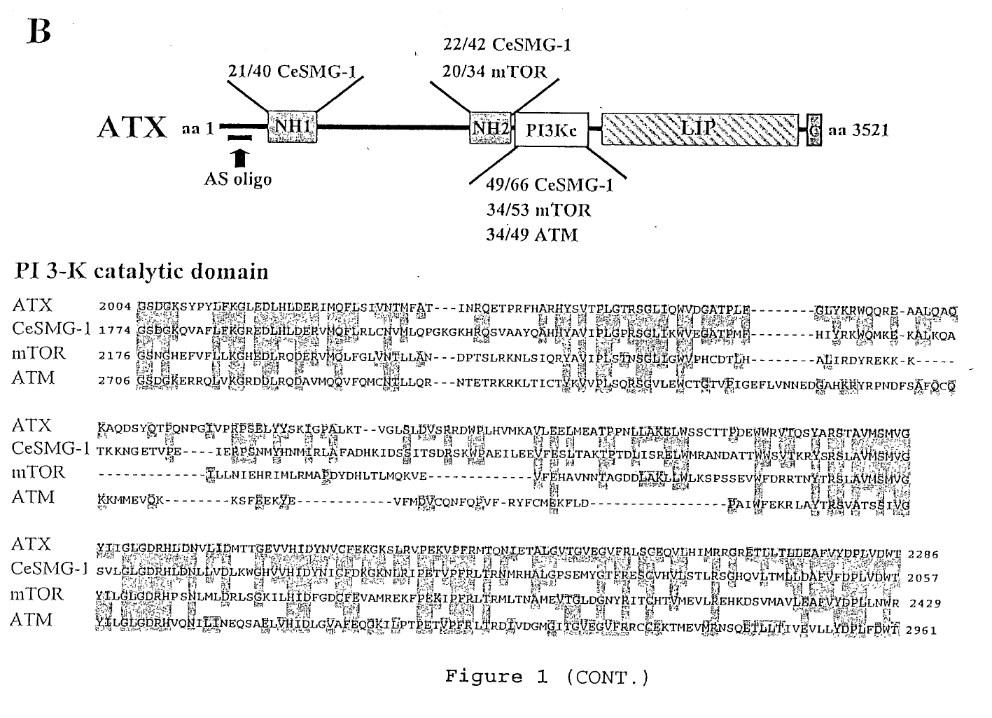

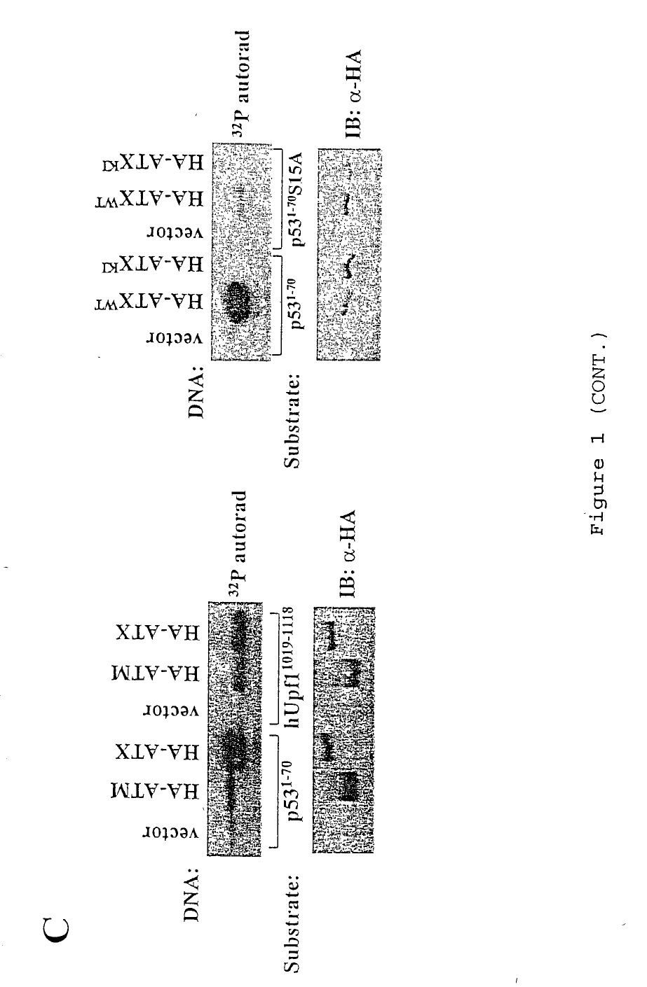

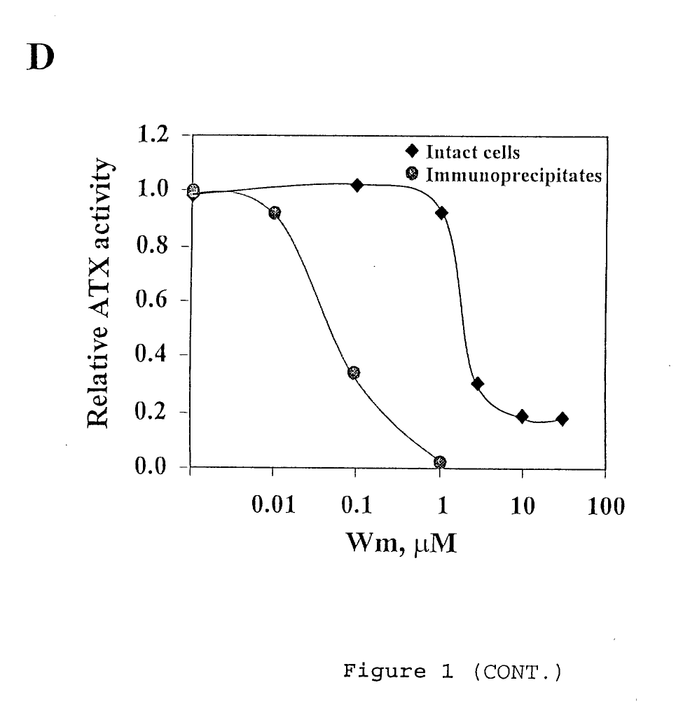

Those skilled in the art can determine which residues and which regions of a ATX sequence are likely to be tolerant of modification and still retain an activity associated with ATX. For example, amino acid substitutions or chemical or enzymatic modifications at residues that are less well conserved between species are more likely to be tolerated than substitutions at highly conserved residues. Accordingly, an alignment can be performed among ATX sequences of various species to determine residues and regions in which modifications are likely to be tolerated (FIG. 1B). Additional guidance for determining residues and regions of ATX likely to be tolerant of modification is provided by studies of ATX fragments and variants. In addition, it can be useful to modify ATX in a way that destroys an activity associated with ATX. For example, as disclosed herein, the mutation of an aspartic acid to an alanine at conserved residue Asp-2195 in the ATX kinase domain generates a kinase-inactive version of ATX.

As used herein, the term “level” in reference to a level of an ATX nucleic acid or polypeptide refers to the amount, accumulation, or rate of synthesis of a molecule or to the amount or rate of an activity associated with the molecule. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a molecule accumulated in a cell, including, for example, the amount of particular post-synthetic modifications of a molecule such as a polypeptide or nucleic acid. In addition, a level can be represented, for example, by the extent of phosphorylation of a substrate molecule or by the amount of an activity such as cell cycle checkpoint activity, NMD activity or ability to induce cell death or cell survival. The term can be used to refer to an absolute amount of a molecule or activity in a sample or to a relative amount of the molecule or activity, including amounts and activities determined under steady-state or non-steady-state conditions. For example, the expression level of a molecule can be determined relative to a control component molecule in a sample.

As used herein, the term “p53” is intended to mean a polypeptide with substantially the same amino acid sequence as that shown in SEQ ID NO:4 (human p53). As described above for ATX, it is understood that p53 includes fragments of the full length p53 polypeptide. For example, the amino terminal 70 amino acids of p53 (p53 1-70) can be used in the methods of the invention as a substrate for ATX kinase activity. Also, for example, a fragment of p53 that includes the LSQE sequence located at amino acids 14 to 17 of p53 can be used as a substrate for ATX kinase activity. In addition, as described above for ATX, a p53 polypeptide includes p53 from species other than humans, and includes modifications to the p53 polypeptide including conservative and non-conservative amino acid changes, post-translational modifications and chemical modification. Also, as described for ATX, a p53 polypeptide can contain additional sequences such as a known epitope or a label moiety.

The term “specifically binds” is intended to mean the molecule will have an affinity for the target molecule that is measurably higher than its affinity for a non-specific interaction. For example, a nucleic acid can specifically bind to another nucleic acid by complementary base pairing between the nucleotides. In addition, a polypeptide such as an antibody that specifically binds another polypeptide will have an affinity for the target polypeptide or antigen that is measurably higher than its affinity for a non-specific interaction. Furthermore, a compound such as a small organic molecule can specifically bind to a target molecule with an affinity that is measurably higher than its affinity for a non-specific interaction. Binding affinity can be low or high affinity so long as the binding is sufficient to be detectable. For example, a compound can bind ATX with a binding affinity (Kd) of about 10⁻⁴ M or less, 10⁻⁵ M or less, 10⁻⁶ M or less, about 10⁻⁷ M or less, including about 10⁻⁸ M or less, such as 10⁻⁹ M or less. Several methods for detecting or measuring nucleotide, polypeptide, and other compound binding are well known in the art and disclosed herein.

As used herein, the term “compound” is intended to mean an isolated macromolecule of natural or synthetic origin that can be assayed using the methods of the invention. A compound includes, for example, a polypeptide, peptidomimetic, non-peptidyl compound, carbohydrate, lipid, an antibody or antibody fragment, a small organic or inorganic molecule, or a nucleotide sequence including an aptamer, antisense oligonucleotide, interfering RNA or ribozyme. For example, a compound can be an isolated cDNA sequence. A compound can have a known or unknown structure. A compound can be isolated or be part of a population of compounds such as a library. For example, a compound can be a small organic compound obtained from a combinatorial chemical library. A library of compounds can be a random collection of compounds or can be rationally designed based on a physical characteristic. A compound which is assayed in the methods of the invention can be called a “test compound” and if the test compound has the ability to modulate the level of ATX it can be called an “ATX-modulatory compound.” One compound or more than one compound can be used in the methods of the invention.

As used herein, a “stressor agent” is any agent that can induce a stress response pathway within a cell. Several stressor agents are known in the art such as UV light, ionizing radiation, reactive oxygen intermediates, cytotoxic agents, and replicational stress imposed by DNA replication inhibitors including, for example, hydroxyurea and aphidicolin. In addition, environmental conditions such as excessive heat can induce a stress response pathway within a cell resulting in, for example, the induction of heat shock proteins. Stress response pathways include DNA repair pathways, non-sense mediated mRNA decay (NMD), heat shock pathways, the induction of apoptosis, activation of the NFkB transcription factor, activation of the stress-activated MAP kinase pathways including, for example, JNK and p38 pathways, and activation of ubiquitin-dependent proteolysis.

As used herein, the term “non-sense mediated messenger RNA (mRNA) decay (NMD)” is intended to mean the surveillance mechanism within cells whereby imperfect mRNAs that contain premature translation termination codons are preferentially degraded. These imperfect mRNAs can result in polypeptides that are nonfunctional or have altered function such as gain-of function or dominant negative mutations.

As used herein, the term an “amount effective” or “effective amount” when used in reference to a compound that modulates cell survival or growth is intended to mean an amount of the compound or molecule sufficient to increase or decrease cell survival or growth. Modulation also includes induction of cell survival or growth or complete blockage of cell survival or growth. In addition, an effective amount of a compound is intended to mean an amount of the compound that is sufficient to treat or reduce the severity of a condition in an affected subject.

The invention provides an isolated nucleic acid molecule having substantially the same nucleotide sequence as SEQ ID NO:1. In addition, the invention provides an isolated nucleic acid molecule having substantially the same nucleotide sequence as SEQ ID NO:1 where the nucleic acid molecule encodes an ATX polypeptide containing an amino acid sequence shown in SEQ ID NO:2. For example, the invention provides an isolated nucleic acid molecule containing the sequence shown in SEQ ID NO:1.

Substantially the same nucleic acid sequence is intended to mean a nucleic acid sequence contains a considerable degree of sequence identity or similarity, such as at least 70%, 80%, 90%, 95%, 98%, or 100% sequence identity or similarity, to a reference nucleic acid sequence. Substantially the same nucleic acid sequence includes nucleic acid changes, gaps, and insertions to an nucleic sequence. Nucleic acid changes, gaps, and insertions to a nucleic acid sequence can be compared to a reference sequence using available algorithms and programs such as the Smith-Waterman algorithm and the BLAST homology search program (Altschul et al., J. Mol. Biol. 215:403-410 (1990)).

Isolated nucleic acid molecules include DNA sequences and RNA transcripts, both sense and complementary anti-sense strands, including splice variants thereof encoding ATX polypeptides. An isolated nucleic acid molecule can contain a double stranded molecules or single stranded molecules, including RNA as well as coding and noncoding DNA. DNA sequences of the invention include genomic and cDNA sequences as well as wholly or partially chemically synthesized DNA sequences. Genomic DNA of the invention comprises the protein coding region for a polypeptide of the invention and includes allelic variants of the preferred nucleic acid of the invention. Genomic DNA of the invention is distinguishable from genomic DNAs encoding polypeptides other than ATX in that it includes an ATX protein coding region found in ATX-encoding cDNA of the invention. Genomic DNA of the invention can be transcribed into RNA, and the resulting RNA transcript can undergo one or more splicing events wherein one or more introns of the transcript are removed, or “spliced out.” Peptide nucleic acids (PNAS) encoding a polypeptide of the invention are also contemplated (Corey, TIBTech 15:224-229 (1997)). PNAs are DNA analogs containing neutral amide backbone linkages that are resistant to DNA degradation enzymes and which bind to complementary sequences at higher affinity than analogous DNA sequences as a result of the neutral charge on the backbone of the molecule.

RNA transcripts that can be spliced by alternative mechanisms, and therefore be subject to removal of different RNA sequences but still encode an ATX polypeptide, are referred to in the art as splice variants which are embraced by the invention. Splice variants comprehended by the invention therefore are encoded by the same DNA sequences but arise from distinct mRNA transcripts. Allelic variants are known in the art to be modified forms of a wild type gene sequence, the modification resulting from recombination during chromosomal segregation or exposure to conditions which give rise to genetic mutation. Allelic variants, like wild type genes, are inherently naturally occurring sequences (as opposed to non-naturally occurring variants which arise from in vitro manipulation).

An allelic variant of ATX is disclosed herein as SEQ ID NO:5. This form of ATX is produced as the result of allelic variation in exon 15 which leads to the insertion of 27 nucleotides beginning at nucleotide 1427 (FIG. 1A). This sequence alteration causes the insertion of two in-frame stop codons and the use of the next available ATG codon in exon 16 as the translational stat site, resulting in an amino-terminally truncated or short form of ATX. A form of ATX that is similar to the long form of ATX disclosed herein (SEQ ID NO:1) is referenced as SEQ ID NO:7. This form of ATX has exon 5 spliced to exon 6 which results in a different N-terminus and 8 additional amino acids in the resulting polypeptide (FIG. 1A). In the experiments disclosed herein clones that were isolated with exon 5 frequently contained exon 3 which place an in-frame stop codon at the 3′ end of this DNA (Example 1). The longest form of ATX (SEQ ID NO:9) was isolated, however the exon 3 associated stop codon was present in this transcript as well.

In addition to genomic DNA, isolated nucleic acids include cDNA. cDNA can be obtained through reverse transcription of an RNA nucleic acid encoding ATX, followed by second strand synthesis of a complementary strand to provide a double stranded DNA. In addition, nucleic acid molecules can be chemically synthesized meaning produced by purely chemical, as opposed to enzymatic, methods. Wholly chemically synthesized DNA sequences are produced entirely by chemical means, and partially synthesized DNAs are those where only portions of the resulting DNA were produced by chemical means.

ATX nucleic acid molecules include homologs of the human ATX sequence. Species homologs in general share at least 50%, at least 60%, at least 70%, at least 80%, at least 90%, at least 95% or at least 99% homology with a human DNA of the invention. ATX nucleic acids include species homologs of the human ATX sequence, but exclude a mouse EST that contains a sequence homologous to the 3′ part of ATX is (GenBank Accession Number BC024431) and a Macaca fascicularis brain cDNA clone Qf1A-15747 (accession number AB056380).

The invention also provides anti-sense oligonucleotides based on SEQ ID NO:1. For example, the invention provides an isolated oligonucleotide having at least 15 contiguous nucleotides of the nucleotide sequence 5′-AGCAAGCTCCCTCCTGTCTC-3′ (SEQ ID NO:11). The oligonucleotide shown in SEQ ID NO:11 is an ATX anti-sense oligonucleotide that has been shown herein to decrease the level of ATX in a cell (Example 5).

Nucleic acids of the invention also permit identification and isolation of nucleic acid encoding related ATX polypeptides by well known techniques including Southern hybridization, Northern hybridization, and polymerase chain reaction (PCR). Examples of related nucleic acids include human and non-human nucleic acid sequences, including allelic variants, as well as nucleic acids encoding polypeptides homologous to ATX and structurally related polypeptides sharing one or more biological, immunological, or physical properties of ATX.

The invention provides a method for detecting an ATX nucleic acid molecule in a sample, by contacting the sample with an ATX nucleic acid molecule under conditions that allow specific hybridization to ATX nucleic acid, and detecting the specific hybridization. In addition, the invention provides a method for detecting an ATX nucleic acid molecule in a sample, by contacting a nucleic acid fraction derived from the sample with a PCR primer pair set under conditions that allow amplification of an ATX nucleic acid, and detecting amplified ATX nucleic acid. Kits for detecting ATX nucleic acids based on these methods are provided as well.

Fragments of ATX nucleic acid molecules are useful in the invention, for example, as probes for detection of full length or other fragment ATX nucleic acids. A nucleic acid fragment can include for example 5′, 3′, or internal deletions of a full length ATX nucleic acid sequence. For example, the invention provides an isolated ATX nucleic acid molecule as referenced in SEQ ID NO:5. Alternatively, the invention provides ATX nucleic acid fragments other than the fragment as referenced in SEQ ID NO:5. For example, the invention provides ATX nucleic acid fragments that contain carboxyl terminal deletions of a full length ATX polypeptide. In addition, fragments can include domains of a full length ATX nucleic acid sequence, for example, a kinase domain, NH1 domain, NH2 domain, or LIP domain. A fragment can contain, for example, at least about 10, 100, 1,000, 2,500, 5,000, 7,500, 10,000, 12,500 or more contiguous or non-contiguous nucleic acid residues of a full-length ATX nucleic acid sequence. ATX nucleic acid fragments include the fragments described above, but excludes fragments KIAA0421 (Accession number AB007881), KIAA0220 (Accession number D86974), and LIP (Accession number U32581), which are present in databases. One or more fragment nucleic acids can be included in kits that are used to detect the presence of a nucleic acids encoding ATX, or used to detect variations in a nucleic acid sequence encoding ATX, including polymorphisms, for example, single nucleotide polymorphisms.

The nucleic acids of the invention can contain heterologous sequences that are not part of the ATX-encoding sequences in nature. The heterologous nucleic acid sequence can be separated from the ATX-coding sequence by an encoded cleavage site that will permit removal of non-ATX polypeptide sequences from the expressed fusion protein. Heterologous nucleic acids sequences can include sequences encoding epitopes, such as poly-histidine sequences, FLAG tags, glutathione-S-transferase, thioredoxin, and maltose binding protein domains, that facilitate purification of the fusion protein. In addition heterologous nucleic acids can encode domains, such as leucine zipper motifs, that promote multimer formation between the fusion protein and itself or other proteins or immunoglobulins or fragments thereof that can enhance circulatory half-life of the encoded protein.

The nucleic acid molecules of the invention also include DNA sequences encoding ATX species that hybridize under highly or moderately stringent conditions to the non-coding strand, or complement, of the nucleic acid in SEQ ID NO: 1. ATX-encoding nucleic acids of the invention include a) the nucleic acid sequence set out in SEQ ID NO: 1; b) nucleic acids encoding a polypeptide encoded by the nucleic acid of (a), and c) nucleic acids that hybridize to the complement of the nucleic acids of (a) or (b) under moderately or highly stringent conditions. Exemplary high stringency conditions include a final wash in 0.2×SSC/0.1% SDS at 65° C. to 75° C., and exemplary moderate stringency conditions include a final wash at 2× to 3×SSC/0.1% SDS at 65° C. to 75° C. It is understood in the art that conditions of equivalent stringency can be achieved through variation of temperature and buffer, or salt concentration as described in Ausubel, et al. (Eds.), Protocols in Molecular Biology, John Wiley & Sons (1994). Modifications in hybridization conditions can be empirically determined or precisely calculated based on the length and the percentage of guanosine/cytosine (GC) base pairing of the probe.

The invention also provides a vector containing the isolated ATX nucleic acid molecules described above. For example, the invention provides a vector containing an isolated nucleic acid molecule having substantially the same nucleotide sequence as SEQ ID NO:1.

Vectors include autonomously replicating recombinant expression constructs such as plasmid and viral DNA vectors. The invention includes vectors where ATX-encoding nucleic acids are operatively linked to an endogenous or exogenous promoter, enhancer, or operator sequence and a transcription terminator sequence. Promoter and enhancer sequences are generally selected for the ability to increase gene expression, while operator sequences are generally selected for the ability to regulate gene expression. It is understood in the art that the choice of host cell is relevant to selection of an appropriate regulatory sequence. Vectors used in the invention can also include sequences encoding one or more selectable markers that permit identification of host cells bearing the construct. Vectors can also include sequences that facilitate homologous recombination in a host cell.

Suitable vectors for expression in prokaryotic or eukaryotic cells are well known to those skilled in the art (see, for example, Ausubel et al., supra, 1999). Vectors useful for expression in eukaryotic cells can include, for example, regulatory elements including the SV40 early promoter, the cytomegalovirus (CMV) promoter, the mouse mammary tumor virus (MMTV) steroid-inducible promoter, Moloney murine leukemia virus (MMLV) promoter, and the like. A vector can include, for example, viral vectors such as a bacteriophage, a baculovirus or a retrovirus; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art. One skilled in the art will know or can readily determine an appropriate promoter for expression in a particular host cell. For example, as disclosed herein, the long form of ATX can be sub-cloned into pcDNA 3.1 with an HA tag and transfected using Fugene 6 into human embryonic kidney 293T cells (Example 2 and Example 5).

Vectors useful for expression of an ATX polypeptide can contain a regulatory element that provides tissue specific or inducible expression of an operatively linked nucleic acid. Such inducible systems, include, for example, tetracycline inducible system (Gossen & Bizard, Proc. Natl. Acad. Sci. USA, 89:5547-5551 (1992); Gossen et al., Science, 268:1766-1769 (1995); Clontech, Palo Alto, Calif.)); metallothionein promoter induced by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse mammory tumor virus (MMTV) induced by steroids such as glucocortocoid and estrogen (Lee et al., Nature, 294:228-232 (1981); and heat shock promoters inducible by temperature changes.

In addition, viral vectors such as retroviral, adenovirus, adeno-associated virus, lentivirus, and herpesvirus vectors can be used to express ATX polypeptides into a cell. Viral based systems provide the advantage of being able to introduce relatively high levels of a heterologous nucleic acid into a variety of cells. Additionally, such viruses can introduce heterologous DNA into nondividing cells. Viral vectors include, for example, Herpes simplex virus vectors (U.S. Pat. No. 5,501,979), Vaccinia virus vectors (U.S. Pat. No. 5,506,138), Cytomegalovirus vectors (U.S. Pat. No. 5,561,063), Modified Moloney murine leukemia virus vectors (U.S. Pat. No. 5,693,508), adenovirus vectors (U.S. Pat. Nos. 5,700,470 and 5,731,172), adeno-associated virus vectors (U.S. Pat. No. 5,604,090), constitutive and regulatable retrovirus vectors (U.S. Pat. Nos. 4,405,712; 4,650,764 and 5,739,018, respectively), papilloma virus vectors (U.S. Pat. Nos. 5,674,703 and 5,719,054), and the like.

The invention further provides a host cell containing an ATX-encoding vector as described above. For example, the invention provides a host cell that contains a vector which contains an isolated nucleic acid molecule having substantially the same nucleotide sequence as SEQ ID NO:1. Host cells include prokaryotic and eukaryotic cells. Nucleic acids of the invention can be introduced into the host cell as part of a circular plasmid, or as linear DNA having an isolated protein coding region or a viral vector. Methods for introducing DNA into the host cell are well known in the art and include transformation, transfection, electroporation, nuclear injection, or fusion with carriers such as liposomes, micelles, ghost cells, protoplasts, and other transformed cells. Detailed procedures for these methods can be found in Sambrook et al., Molecular Cloning: A Laboratory Manual (Cold Spring Harbor Laboratory Press, 1989) and the references cited therein). Expression systems of the invention include bacterial, yeast, fungal, plant, insect, invertebrate, and mammalian cells systems.

Useful mammalian expression vectors and methods of introducing such vectors into mammalian cells either ex vivo or in vivo, for expression of the encoded polypeptide, are well known in the art. For example, a plasmid expression vector can be introduced into a cell by calcium-phosphate mediated transfection, DEAE-Dextran-mediated transfection, lipofection, polybrene- or polylysine-mediated transfection, electroporation, or by conjugation to an antibody, gramacidin S, artificial viral envelopes or other intracellular carriers. A viral expression vector can be introduced into a cell in an expressible form by infection or transduction, for example, or by encapsulation in a liposome.

The invention also provides a method of producing an ATX polypeptide by a) growing the host cell described above under conditions appropriate for expression of the ATX polypeptide, and b) isolating the ATX polypeptide from the host cell or host cell growth medium. This method can be used to produce ATX polypeptide, for example, as a source of immunogen for the development of antibodies specifically reactive with ATX.

ATX polypeptide isolated from the cells or from the medium in which the cells are grown by purification methods known in the art, for example, conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those wherein the desired protein is expressed and purified as a fusion protein having a specific tag, label, or chelating moiety that is recognized by a specific binding partner or agent. The purified protein can be cleaved to yield the desired protein, or be left as an intact fusion protein.

The DNA sequence information provided by the present invention also makes possible the development through, for example, homologous recombination or “knock-out” strategies of animals that fail to express functional ATX or that express a variant of ATX (Capecchi, Science 244:1288-1292 (1989)). Such animals are useful as models for studying the in vivo activities of ATX and modulators of ATX.

The invention provides an isolated polypeptide containing substantially the same amino acid sequence as SEQ ID NO:2. For example, the invention provides a polypeptide containing an amino acid sequence as referenced in SEQ ID NO:2. The sequence shown in SEQ ID NO:2 corresponds to the “long form” of ATX (FIG. 1A).

As described further above, an isolated ATX polypeptide includes conservative and non-conservative amino acid changes to the sequence shown in SEQ ID NO:2. In addition, an isolated ATX polypeptide includes species homologs and fragments of ATX. For example, the invention provides an isolated ATX polypeptide fragment as referenced in SEQ ID NO:6. Alternatively, the invention provides ATX polypeptide fragments other than the fragment as referenced in SEQ ID NO:6. For example, the invention provides ATX polypeptide fragments that contain carboxyl terminal deletions of a full length ATX polypeptide. Furthermore, an ATX polypeptide can contain polypeptide modifications or heterologous sequences such as an epitope tag. Polypeptides of the invention can be isolated from natural cell sources, chemically synthesized, or produced by recombinant procedures involving the host cells of the invention.

The invention provides an antibody, or antigen binding fragment thereof, which specifically binds to an ATX polypeptide containing an amino acid sequence as referenced in SEQ ID NO:2. Antibodies include, for example, monoclonal and polyclonal antibodies, single chain antibodies, chimeric antibodies, bifunctional or bispecific antibodies, humanized antibodies, human antibodies, and complementary determining region (CDR)-grafted antibodies, including compounds which include CDR or antigen-binding sequences, which specifically bind to a polypeptide of the invention. Antibody fragments, including Fab, Fab′, F(ab′)₂, and Fv, are also provided by the invention. Screening assays to determine binding specificity or exclusivity of an antibody of the invention are well known in the art (see Harlow et al. (Eds), Antibodies A Laboratory Manual; Cold Spring Harbor Laboratory; Cold Spring Harbor, N.Y. (1988)).

Antibodies that recognize and bind fragments of the ATX polypeptides of the invention are also contemplated, provided that the antibodies specifically bind ATX polypeptides. As with antibodies that are specific for full length ATX polypeptides, antibodies of the invention that recognize ATX fragments are those which can distinguish ATX polypeptides from other PIKK polypeptides despite inherent sequence identity, homology, or similarity found in the family of proteins.

Antibodies of the invention can be produced using any method well known in the art, using any polypeptide, or immunogenic fragment thereof, of the invention. Immunogenic polypeptides can be isolated from natural sources, from recombinant host cells, or can be chemically synthesized. For example, as disclosed herein, antibodies specifically reactive with ATX were generated using glutathione S-transferase (GST) fusion proteins containing ATX amino acids 2281-2339 (anti-ATX-Ab-1) or amino acids 1691-1790 (anti-ATX-Ab-2) (Example 2). Polypeptide of the invention can also be conjugated to a hapten such as keyhole limpet hemocyanin (KLH) in order to increase immunogenicity. Methods for synthesizing such peptides are known in the art, for example, as in R. P. Merrifield, J. Amer. Chem. Soc. 85: 2149-2154 (1963); J. L. Krstenansky, et al., FEBS Lett. 211:10 (1987). Antibodies to a polypeptide of the invention can also be prepared through immunization using a nucleic acid of the invention, as described in Fan et al., Nat. Biotech. 17:870-872 (1999). DNA encoding a polypeptide can be used to generate antibodies against the encoded polypetide following topical administration of naked plasmid DNA or following injection, for example, intramuscular injection, of the DNA.

Non-human antibodies can be humanized by any methods known in the art. In one method, the non-human CDRs are inserted into a human antibody or consensus antibody framework sequence. Further changes can then be introduced into the antibody framework to modulate affinity or immunogenicity. Antibodies of the invention further include plastic antibodies or molecularly imprinted polymers (MIPs) (Haupt and Mosbauch, TIBTech 16:468-475 (1998)). Antibodies of this type can be useful in immunoaffinity separation, chromatography, solid phase extraction, immunoassays, for use as immunosensors, and for screening chemical or biological libraries. Advantages of antibodies of this type are that no animal immunization is required, the antibodies are relatively inexpensive to produce, they are resistant to organic solvents, and they are reusable over long period of time.

The invention provides a method for detecting ATX polypeptide in a sample by contacting the sample with an ATX antibody under conditions that allow specific binding of the antibody to the polypeptide and detecting the bound antibody. Antibodies of the invention can also include one or more labels that permit detection of the antibody and antibody binding. Labels can include, for example, radioactivity, fluorescence (or chemiluminescence), one of a high affinity binding pair (such as biotin/avidin), enzymes, or combinations of one or more of these labels. Antibodies of the invention are also useful, for example, for therapeutic purposes (by modulating activity of ATX), diagnostic purposes to detect or quantitate ATX, as well as purification of ATX. Kits containing an antibody or antibodies of the invention are also provided.

The DNA and amino acid sequence information provided by the present invention also makes possible the systematic analysis of the structure and function of ATX. DNA and amino acid sequence information for ATX also permits identification of compounds with which an ATX polypeptide or nucleic acid will interact. Methods to identify compounds that bind to ATX include solution assays, in vitro assays where ATX polypeptides are immobilized, and cell based assays. Identification of compounds that bind ATX polypeptides provides potential targets for therapeutic or prophylactic intervention in pathologies associated with ATX biological activity.

The invention provides a method for identifying a compound that specifically binds to an ATX polypeptide of the invention, by a) contacting the ATX polypeptide with a compound, and b) determining specific binding of the compound to said ATX polypeptide. As described further above, the term compound includes macromolecules of natural or synthetic origin including, for example, a polypeptide, peptidomimetic, non-peptidyl compound, carbohydrate, lipid, and antibody or antibody fragment, a small organic or inorganic molecule, or a nucleic acid including an aptamer.

Identification of compounds that bind the ATX polypeptide can be achieved by isolating the ATX polypeptide/binding complex, and separating the ATX polypeptide from the binding compound. An additional step of characterizing the physical, biological, or biochemical properties of the binding compound can also be performed. In one embodiment, the ATX polypeptide/binding complex can be isolated using a antibody immunospecific for either the ATX polypeptide or the candidate binding compound. In another embodiment, the complex can be isolated using a second binding compound that interacts with either the ATX polypeptide or the candidate binding compound. In still another embodiment, either the polypeptide ATX or the candidate binding compound comprises a label or tag that facilitates its isolation, and methods of the invention to identify binding compounds include a step of isolating the ATX polypeptide/binding complex through interaction with the label or tag. An exemplary tag of this type is a poly-histidine sequence, generally around six histidine residues, that permits isolation of a compound so labeled using nickel chelation. Other labels and tags, such as the FLAG tag, thioredoxin, and GST, each of which is well known in the art.

An in vitro assay can be performed where the ATX polypeptide can be immobilized and then contacted with a candidate binding compound. In an alternative embodiment, the candidate binding compound can be immobilized and binding of the ATX polypeptide is detected. Immobilization can be accomplished using any of the methods well known in the art, including covalent bonding to a support, a bead, or a chromatographic resin, as well as non-covalent, high affinity interaction such as antibody binding, or use of streptavidin/biotin binding wherein the immobilized compound includes a biotin or streptavidin moiety. Detection of binding can be accomplished, for example, (i) using a radioactive label on the compound that is not immobilized, (ii) using of a fluorescent label on the non-immobilized compound, (iii) using an antibody immunospecific for the non-immobilized compound, (iv) using a label on the non-immobilized compound that excites a fluorescent support to which the immobilized compound is attached, as well as other techniques well known in the art.

A cell based assay that can be used in the method of the invention for detecting an ATX binding compound is a yeast or mammalian two-hybrid assay (Fields and Song, Nature 340:245-246 (1989); Fields, Methods: A Companion to Methods in Enzymology 5:116-124 (1993); U.S. Pat. No. 5,283,173 issued Feb. 1, 1994 to Fields, et al.). Modifications and variations on the two-hybrid assay have previously been described (Colas and Brent, TIBTECH 16:355-363 (1998)).

The invention also provides a method for identifying an ATX-modulatory compound by measuring the level of an ATX polypeptide in the presence of a test compound, where a difference in the level of the ATX polypeptide in the presence of the test compound compared to in the absence of the test compound indicating that the test compound is an ATX-modulatory compound. In addition, the invention provides a method for identifying an ATX-modulatory compound by measuring the level of an ATX polypeptide in the presence of a test compound, where a difference in the level of the ATX polypeptide in the presence of the test compound compared to in the absence of the test compound indicating that the test compound is an ATX-modulatory compound, and where the ATX-modulatory compound is not caffeine or wortmannin. The ATX-modulatory compound can decrease or increase the level of ATX polypeptide.

Agents that modulate, for example, increase, decrease, or block the level of ATX can be identified by incubating a test compound with an ATX polypeptide or nucleic acid and determining the effect of the test compound on ATX activity or expression. The level of ATX can include the expression level of ATX or an activity level of ATX. The selectivity, or specificity, of an ATX-modulatory compound can be evaluated by comparing its effects on ATX or an ATX-encoding nucleic acids to its effect on other polypeptides or compounds. Cell based methods, such as two-hybrid assays to identify DNAs encoding binding compounds and split hybrid assays to identify inhibitors of ATX polypeptide interaction with a known binding polypeptide, as well as in vitro methods, including assays where an ATX polypeptide, ATX-encoding nucleic acid, or a binding compound are immobilized, and solution assays are included in this method of the invention.

As understood by those of skill in the art, assay methods for identifying compounds that modulate an activity generally require comparison to a “control.” One type of a control is a reaction or cell that is treated substantially the same as the test reaction or cell exposed to the compound, with the distinction that the control reaction or cell is not exposed to the compound.

As disclosed herein, the compounds wortmannin and caffeine can modulate (inhibit) the level of ATX (Example 3 and Example 7). Wortmannin is known to inhibit ATM kinase and is an irreversible inhibitor of PIKKs. Caffeine is a known inhibitor of the G2 cell cycle checkpoint. As disclosed herein, caffeine reversed the accumulation of G2/M phase cells induced by ATX anti-sense treatment, indicating that ATX deficiency can trigger the activation of a caffeine-sensitive G2 checkpoint (Example 7).

The invention provides a method for identifying an ATX-modulatory compound where the level of ATX polypeptide is measured by determining the kinase activity of the ATX polypeptide. The kinase activity of ATX can be measured using methods well known in the art such as kinase assays and immune complex kinase assays as performed herein in Example 3. These assays contain ATX, a substrate, and a suitable buffer including [g-32 P]ATP and Mn²⁺. Phosphorylated substrates can also be detected using phospho-specific antibodies.

In addition, the invention provides a method for identifying an ATX-modulatory compound where the level of ATX polypeptide is measured by determining the phosphorylation of a p53 polypeptide or fragment. For example, a GST fusion protein containing the first 70 amino acids of p53 (GST-p53₁₋₇₀) can be used as a substrate to measure the level of ATX polypeptide by its kinase activity (Example 3). In addition to p53, the phosphorylation of hUpf1, a helicase, can be used to measure the level of ATX polypeptide (Example 3).

The invention also provides a method for identifying an ATX-modulatory compound where the level of ATX polypeptide is measured by determining the level of p53 polypeptide accumulation. As shown herein, a decrease in ATX polypeptide, such as results from the use of an anti-sense oligonucleotide, leads to a reduction in p53 polypeptide accumulation (Example 6). Thus, the level of p53 can be used as a measure of ATX polypeptide level.

The invention further provides a method for identifying an ATX-modulatory compound where the level of ATX polypeptide is measured by determining the level of non-sense mediated messenger RNA (mRNA) decay (NMD). NMD is a surveillance mechanism within cells whereby imperfect mRNAs that contain premature translation termination codons are preferentially degraded. As disclosed herein, treatment of cells with an ATX anti-sense oligonucleotide, which reduced endogenous ATX expression, demonstrated that ATX expression is required for maximal NMD activity (Example 9). The level of NMD is correlated to the level of ATX in the cell and so the level of NMD can be used as a measure of ATX polypeptide level.

ATX-modulatory compounds can be identified that decrease or increase the level of ATX polypeptide or nucleic acid. A decrease in the level of ATX can be a partial reduction or a total blockage of the level of ATX, and an increase in the level of ATX can be a partial increase or an induction of the level of ATX from a previously undetectable level. For example, an ATX-modulatory compound can increase the level of NMD activity in a cell. It can be desirable to increase the level of NMD activity in a cell in order to protect the cell from deleterious gain-of-function mutations caused by truncated polypeptides resulting from the translation of imperfect mRNAs that contain premature translation termination. Alternatively, an ATX-modulatory compound can decrease the level of NMD activity in a cell. It can be desirable to decrease the level of NMD activity in a cell in some cases where the truncated polypeptide does not have a deleterious effect but instead retains some activity that can compensate for the normal gene function.

ATX-modulatory compounds can include, for example, antibodies and other proteins or peptides which specifically bind to an ATX polypeptide or an ATX-encoding nucleic acid, oligonucleotides which bind to an ATX polypeptide or an ATX gene sequence, and other non-peptide compounds, for example, isolated or synthetic organic and inorganic molecules, which specifically react with an ATX polypeptide or underlying nucleic acid. ATX-modulatory compounds of the invention can interact specifically or exclusively to an ATX polypeptide or ATX-encoding nucleic acid, however, modulators that interact with an ATX polypeptide or an ATX-encoding nucleic acid with higher affinity or avidity compared to other compounds are also included in the invention. Mutant ATX polypeptides which affect the enzymatic activity or cellular localization of the wild-type ATX polypeptides are also contemplated by the invention. Targets for the development of ATX-modulatory compounds can include, for example: (1) regions of an ATX polypeptide which contact other proteins, (2) regions that localize an ATX polypeptide within a cell, (3) regions of an ATX polypeptide which bind substrate, (4) allosteric regulatory binding site(s) of an ATX polypeptide, (5) phosphorylation site(s) of an ATX polypeptide as well as other regions of the protein where covalent modification regulates biological activity and (6) regions of an ATX polypeptide which are involved in multimerization of subunits. Still other ATX-modulatory compounds include those that recognize specific ATX-encoding and regulatory nucleic acid sequences. ATX-modulatory compounds that modulate the level of ATX can be therapeutically useful in treatment of diseases and physiological conditions in which ATX is known or suspected to be involved.

Methods of the invention to identify ATX-modulatory compounds include variations on any of the methods described above to identify ATX binding compounds, the variations including techniques where a binding compound has been identified and the binding assay is carried out in the presence and absence of a candidate ATX-modulatory compound. A modulatory compound is identified in those instances where the level of binding between an ATX polypeptide and a binding compound changes in the presence of the candidate modulatory compound compared to the level of binding in the absence of the candidate modulatory compound. An ATX-modulatory compound that increases binding between an ATX polypeptide and the binding compound is described as an enhancer or activator, and a modulatory compound that decreases binding between the ATX polypeptide and the binding compound is described as an inhibitor. In vitro methods of the invention are amenable to high throughput assays as described below.

In addition to the assays described above which can be modified to identify binding compounds, other methods are contemplated to identify modulatory compounds. In one embodiment, methods of the invention can include use of the split hybrid assay as generally described in WO98/13502 and variations on this method as described in WO95/20652.

The methods of the invention can also utilize high throughput screening (HTS) assays to identify compounds that interact with or inhibit biological activity of an ATX polypeptide. HTS assays permit screening of large numbers of compounds in an efficient manner. Cell-based HTS systems include melanophore assays, yeast-based assay systems, and mammalian cell expression systems (Jayawickreme and Kost, Curr. Opin. Biotechnol. 8:629-634 (1997)). Automated (robotic) and miniaturized HTS assays are also embraced (Houston and Banks, Curr. Opin. Biotechnol. 8:734-740 (1997)). HTS assays are designed to identify “hits” or “lead compounds” having the desired property, from which modifications can be designed to improve the desired property. Chemical modification of the “hit” or “lead compound” is often based on an identifiable structure/activity relationship (SAR) between the “hit” and the ATX polypeptide.

There are a number of different libraries used for the identification of small molecule modulators, including, (1) chemical libraries, (2) natural product libraries, and (3) combinatorial libraries comprised of random peptides, oligonucleotides or organic molecules.

Chemical libraries consist of structural analogs of known compounds or compounds that are identified as “hits” or “leads” via natural product screening. Natural product libraries are collections from microorganisms, animals, plants, or marine organisms which are used to create mixtures for screening by, for example, (1) fermentation and extraction of broths from soil, plant or marine microorganisms or (2) extraction of plants or marine organisms. Natural product libraries include polyketides, non-ribosomal peptides, and variants (non-naturally occurring) variants thereof. Combinatorial libraries are composed of large numbers of peptides, oligonucleotides or organic compounds as a mixture. They can be prepared by traditional automated synthesis methods, PCR, cloning or proprietary synthetic methods. Libraries that can be utilized by the invention include peptide and oligonucleotide combinatorial libraries. Still other libraries of interest include protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Anti-sense oligonucleotides which recognize and hybridize to nucleic acid encoding ATX can also be utilized by the methods of the invention. Full length and fragment anti-sense oligonucleotides are provided. One skilled in the art of will appreciate that fragment anti-sense molecules of the invention include (i) those which specifically or exclusively recognize and hybridize to ATX-encoding RNA (as determined by sequence comparison of DNA encoding ATX to DNA encoding other molecules) as well as (ii) those which recognize and hybridize to RNA encoding variants of the ATX family of proteins. Antisense oligonucleotides that hybridize to RNA encoding other members of the PIKK family of proteins are also identifiable through sequence comparison to identify characteristic, or signature, sequences for the family of molecules. Identification of sequences unique to ATX-encoding nucleic acids, as well as sequences common to the family of PIKK-encoding nucleic acids, can be deduced through use of any publicly available sequence database, or through use of commercially available sequence comparison programs. After identification of the desired sequences, isolation through restriction digestion or amplification using any of the various polymerase chain reaction techniques well known in the art can be performed. Anti-sense oligonucleotides can be used for regulating expression of ATX by those cells expressing ATX mRNA. Antisense molecules are generally from about 5 to about 100 nucleotide in length, and preferably are about 10 to 20 nucleotides in length. Antisense nucleic acids capable of specifically binding to ATX expression control sequences or ATX RNA are introduced into cells, for example, by a viral vector or colloidal dispersion system such as a liposome.

The anti-sense nucleic acid binds to the ATX-encoding target nucleotide sequence in the cell and prevents transcription or translation of the target sequence. Phosphorothioate and methylphosphonate anti-sense oligonucleotides are specifically contemplated for therapeutic use by the invention. The anti-sense oligonucleotides may be further modified by poly-L-lysine, transferrin polylysine, or cholesterol moieties at their 5′ end.

The invention also provides methods to modulate ATX expression through the use of RNA interference (RNAi) (Brummelkamp et al., Science 296:550-553 (2002); Elbashir et al., Nature 411:494-498 (2002)). RNAi is a process of sequence-specific gene silencing by post-transcriptional RNA degradation, which is initiated by double-stranded RNA (dsRNA) homologous in sequence to the silenced gene. A double-stranded RNA (dsRNA) that is used for RNAi is referred to herein as an “interfering RNA.” For example, a suitable dsRNA for RNAi can contain sense and antisense strands of about 21 contiguous nucleotides corresponding to the gene to be targeted that form 19 RNA base pairs, leaving overhangs of two nucleotides at each 3′ end (Elbashir et al., supra; Bass, Nature 411:428-429 (2001); Zamore, Nat. Struct. Biol. 8:746-750 (2001)). dsRNAs of about 25-30 nucleotides have also been used successfully for RNai (Karabinos et al., Proc. Natl. Acad. Sci. 98:7863-7868 (2001). dsRNA can be synthesized in vitro and introduced into a cell by methods known in the art. By using RNAi methods, the targeted RNA is degraded, and translation of the target polypeptide is decreased or abolished.

The invention further provides methods to modulate ATX expression through the use of ribozymes (Gibson and Shillitoe, Mol. Biotech. 7:125-137 (1997)). Ribozyme technology can be utilized to inhibit translation of ATX mRNA in a sequence specific manner through (i) the hybridization of a complementary RNA to a target mRNA and (ii) cleavage of the hybridized mRNA through nuclease activity inherent to the complementary strand. Ribozymes can be identified by empirical methods or be specifically designed based on accessible sites on the target mRNA (Bramlage, et al., Trends in Biotech 16:434-438 (1998)). Delivery of ribozymes to target cells can be accomplished using either exogenous or endogenous delivery techniques well known in the art. Exogenous delivery methods can include use of targeting liposomes or direct local injection. Endogenous methods include use of viral vectors and non-viral plasmids. Ribozymes can be ATX-modulatory compounds and specifically modulate expression of ATX when designed to be complementary to regions unique to a nucleic acid encoding ATX. Specifically modulate means that ribozymes of the invention exclusively recognize a nucleic acid encoding ATX.

The invention further provides methods to modulate transcription of ATX through use of oligonucleotide-directed triple helix formation (Lavrovsky, et al., Biochem. Mol. Med. 62:11-22 (1997)). Triple helix formation is accomplished using sequence specific oligonucleotides which hybridize to double stranded DNA in the major groove as defined in the Watson-Crick model. Hybridization of a sequence specific oligonucleotide can thereafter modulate activity of DNA-binding proteins, including, for example, transcription factors and polymerases. Target sequences for hybridization include promoter and enhancer regions to permit transcriptional regulation of ATX expression. In addition to use of oligonucleotides, triple helix formation techniques of the invention also include use of peptide nucleic acids as described in Corey, TIBTECH 15:224-229 (1997). Oligonucleotides which are capable of triple helix formation are also useful for site-specific covalent modification of target DNA sequences. Oligonucleotides useful for covalent modification can be coupled to various DNA damaging agents as described in Lavrovsky, et al. (supra)

Mutations in the ATX gene can result in loss of normal function of the ATX gene product and underlie ATX-related human disease states. The invention therefore provides gene therapy methods to restore ATX activity in treating those disease states described herein. Delivery of a functional ATX gene to appropriate cells is effected ex vivo, in situ, or in vivo by use of vectors, for example, viral vectors such as adenovirus, adeno-associated virus, or a retrovirus, or ex vivo by use of physical DNA transfer methods such as liposomes or chemical treatments (Anderson, Nature, supplement to vol. 392, no. 6679, pp.25-20 (1998)). Alternatively, in some human disease states, preventing the expression of, or inhibiting the activity of, ATX can be useful in treating the disease states. In this case, anti-sense therapy or gene therapy, for example, where a dominant negative ATX mutant is introduced into a target cell type, can be applied to negatively regulate the expression of ATX.

The invention provides a method for modulating cell survival by introducing an ATX-modulatory compound identified by the methods described above into a cell in an amount effective to modulate survival of the cell. For example, the ATX-modulatory compound can decrease or increase cell survival.

A level of cell death or cell survival can be measured by any of a variety of methods known to one skilled in the art. For example, trypan blue staining can be used to measure the level of cell death in a cell. In addition, clonogenic assays, as described herein, can be used (Example 5). Other staining methods, for example, propidium iodide and Alomar Blue, also can be used to measure cell death. The stained cells can be visualized in any way that is convenient, for example, by microscopy or flow cytometry (FACS). In addition, cell viability and cell proliferation assays such as the lactose dehydrogenase (LDH) assay and the MTT (3-(4,5-dimethylthiazolyl-2)-2,5-diphenyltetrazolium bromide) assay are commercially available and can be used to measure cell viability. In addition, the uptake of 3H thymidine can be used to access the viability of cells.

The invention further provides a method for modulating cell survival by introducing an ATX-modulatory compound into a cell where the cell is exposed to a stressor agent. As described further above, a stressor agent is any agent that can induce a stress response pathway within a cell. A stressor agent can include, for example, UV light, ionizing radiation, reactive oxygen intermediates, or a chemical agent such as a cytotoxic or chemotherapeutic agent. In addition, environmental conditions such as excessive heat can induce a stress response pathway within a cell resulting in, for example, the induction of heat shock proteins. Stress response pathways include DNA repair pathways, non-sense mediated mRNA decay (NMD), heat shock pathways, the induction of apoptosis, activation of the NFkB transcription factor, activation of the stress-activated MAP kinase pathways including, for example, JNK and p38 pathways, and activation of ubiquitin-dependent proteolysis.

An example of an ATX-modulatory compound of the invention is an antisense oligonucleotide. The invention provides a method for decreasing cell survival by introducing an antisense oligonucleotide, such as SEQ ID NO: 11 into a cell in an amount effective to decrease survival of the cell.

Association of ATX with cell cycle progression makes compositions of the invention, including for example an ATX polypeptide, an inhibitor thereof, an antibody, or other modulator of ATX expression or biological activity, useful for treating a number of conditions. For example, the invention provides a method for treating a condition characterized by excessive cell survival or cell growth by administering to a patient having such a condition an effective amount of an ATX-modulatory compound where the effective amount of the compound increases cell death. For example, an ATX-modultory compound can be given to a patient with a neoplastic condition.

An ATX-modulatory compound that decreases the level of ATX can enhance the radiosensitivity or chemosensitivity of neoplastic cells. Therefore, it is contemplated that an ATX-modulatory compound can be given alone or in combination with another agent such as a cytotoxic or chemotherapeutic agent. Several cytotoxic agents, such as radiation, and chemotherapeutic agents, such as cis-platin, are well known in the art. An appropriate agent can be chosen based on several factors, such as the particular type of neoplastic condition at issue or the ability of the patient to tolerate the agent. For example, focused radiation therapy, including brachytherapy, can be used in conjunction with an ATX inhibitory compound in order to induce tumor cell death while minimizing cytotoxic effects on normal tissue.

A “neoplastic condition,” refers to a condition associated with hyperproliferation of cells and includes benign and malignant expanding lesions of proliferating cells. Neoplastic conditions include benign and malignant hyperproliferative disorders. A benign neoplasm grows in an expansile manner, displacing or compressing surrounding tissues rather than invading them. A malignant neoplasm refers to a large group of diseases characterized by uncontrolled growth and spread of abnormal cells. Cancer, for example, is a malignant neoplastic condition that encompasses many sub-conditions that are characterized by insufficient death of abnormal cells. Tumors of the colon, prostate, lung, cervix, stomach, breast and skin are examples of neoplastic conditions.

Aberrant ATX activity can be associated with various forms of cancer in, for example, adult and pediatric oncology, including growth of solid tumors/malignancies, myxiod and round cell carcinoma, locally advanced tumors, metastatic cancer, human soft tissue sarcomas, cancer metastases, including lymphatic metastases, squamous cell carcinoma of the head and neck, esophageal squamous cell carcinoma, oral carcinoma, blood cell malignancies, including multiple myeloma, leukemias, effusion lymphomas (body cavity based lymphomas), thymic lymphoma lung cancer, including small cell carcinoma, non-small cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, and solid tumors in the ovarian follicle, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas, skin cancers, including malignant melanoma, tumor progression of human skin keratinocytes, and squamous cell cancer, hemangiopericytoma, and Kaposi's sarcoma.

Aberrant ATX activity also can be associated with other conditions which include aberrant apoptotic mechanisms, including abnormal caspase activity; aberrant enzyme activity associated with cell cycle progression, including for example cyclins A, B, D and E; alterations in viral (such as Epstein-Barr virus, papillomavirus) replication in latently infected cells; chromosome structure abnormalities, including genomic stability in general, unrepaired chromosome damage, telomere erosion (and telomerase activity), breakage syndromes including for example, Sjogren's syndrome and Nijimegen breakage syndrome; embryonic stem cell lethality; abnormal embyonic development; sensitivity to ionizing radiation; acute immune complex alveolitis; and Fanconi anemia. ATX-modulatory compounds can be used alone or in combination with another agent in the treatment of these conditions.

The invention also provides a method for treating a condition characterized by excessive cell death by administering to a patient having such a condition an effective amount of an ATX-modulatory compound where the effective amount of the compound increases cell survival. For example, an ATX-modultory compound can be given to a patient with a neurodegnerative condition in order to increase neuronal cell survival. In addition the invention provides a method of prolonging the in vivo survival of transplanted cells for the treatment of a disease or pathological condition. Also, for example, a compound that increases the level of ATX can be given to a patient who is exposed to stressors such as UV light in order to protect against genetic mutations.

The effective compounds of the invention described herein can optionally be formulated together with a pharmaceutically acceptable carrier for delivery to a cultured cell or to a subject. Suitable pharmaceutically acceptable carriers are well known in the art and include, for example, aqueous or organic solvents such as physiologically buffered saline, glycols, glycerol, oils or injectable organic esters. A pharmaceutically acceptable carrier can also contain a physiologically acceptable compound that acts, for example, to stabilize or increase the solubility of a pharmaceutical composition. Such a physiologically acceptable compound can be, for example, a carbohydrate, such as glucose, sucrose or dextrans; an antioxidant, such as ascorbic acid or glutathione; a chelating agent; a low molecular weight polypeptide; or another stabilizer or excipient. Pharmaceutically acceptable carriers, including solvents, stabilizers, solubilizers and preservatives, are described, for example, in Martin, Remington's Pharm. Sci., 15th Ed. (Mack Publ. Co., Easton, 1975).

Those skilled in the art can formulate the therapeutic molecules to ensure proper distribution in vivo. For example, the blood-brain barrier (BBB) excludes many highly hydrophilic compounds. To ensure that the effective compounds of the invention cross the BBB, if desired, they can be formulated, for example, in liposomes, or chemically derivatized. For a review of strategies for increasing bioavailability of polypeptide drugs in the brain, and of methods for determining the permeability of polypeptides through the BBB using in vitro and in vivo assays, see Engleton et al., Peptides 9:1431-1439 (1997). Strategies that have been successfully used to increase the permeability of other neuropeptides through the BBB are particularly contemplated. Modifications to a polypeptide of the invention that can increase its BBB penetration include conjugating the peptide to a lipophilic moiety, such as a lipophilic amino acid or methyldihydropyridine. Similar modifications to invention polypeptides or peptidomimetics are likewise expected to be advantageous.

Methods of ensuring appropriate distribution in vivo can also be provided by rechargeable or biodegradable devices, particularly where gradients of concentrations of drug in a tissue are desired. Various slow release polymeric devices are known in the art for the controlled delivery of drugs, and include both biodegradable and non-degradable polymers and hydrogels. Those skilled in the art understand that the choice of the pharmaceutical formulation and the appropriate preparation of the composition will depend on the intended use and mode of administration.

The effective compounds of the invention can be administered to a subject by any effective route. Suitable routes for delivering the therapeutic molecules of the invention include topically, intraocularly, intradermally, parenterally, orally, intranasally, intravenously, intramuscularly, intraspinally, intracerebrally and subcutaneously. The present invention also provides compounds containing an acceptable carrier such as any of the standard pharmaceutical carriers, including phosphate buffered saline solution, water and emulsions such as an oil and water emulsion, and various types of wetting agents.

An effective dose of an effective compound of the invention can be determined, for example, by extrapolation from the concentration required in the ATX binding or ATX activity assays described herein; or from the dose required to modulate cell proliferation. An effective dose of an effective compound of the invention for the treatment of a pathology can also be determined from appropriate animal models, such as transgenic mice. Animal models for pathologies such as tumors are well-known in the art. An effective dose for treating this disease is a dose that results in either partial or complete regression of the tumor, reduction in metastasis, reduced discomfort, or prolonged life span. The appropriate dose for treatment of a human subject with a therapeutic molecule of the invention can be determined by those skilled in the art, and is dependent on the nature and bioactivity of the particular compound, the desired route of administration, the gender, age and health of the individual, the number of doses and duration of treatment, and the particular condition being treated.

It is understood that modifications which do not substantially affect the activity of the various embodiments of this invention are also included within the definition of the invention provided herein. Accordingly, the following examples are intended to illustrate but not limit the present invention.

### EXAMPLE 1

### Molecular Cloning of ATX

This example shows the cloning of ATX nucleic acids. During a BLAST search for mTOR-related proteins, it was noted that an expressed sequence tag (EST) (KIAA0421) contained a 5′-terminus with an open reading frame (ORF) that bore clear homology to a conserved region in the catalytic domains of PIKK family members. To access the full-length cDNA, the EST was used to generate a primer for 5′-RACE with human brain cDNA as the template. The initial 5′-RACE product extended the region of homology with the PIKK catalytic domain. Sequential screens of human brain (Clontech #HL1128a) and Jurkat T cell cDNA libraries (Stratagene #936219), combined with 5′-RACE of brain and Jurkat cDNA, resulted in the isolation of several overlapping DNA fragments that were assembled into approximately 12 kb of contiguous nucleotide sequence. This cDNA contains an ORF of 10,563 nucleotides with an additional 1.8 kb of 3′-UTR, and encodes a 3,521 amino acid polypeptide with a deduced molecular mass of 395 kDa. The first nucleotide of the ATG translation initiation codon in exon 6 as has been designated as nucleotide “1”, and nucleotides upstream of this ATG are identified in the 3′ to 5′ direction with negative numbers. The conclusion that this sequence was derived from a single mRNA transcript was confirmed by PCR with primers that were complementary to the extreme 5′-terminus (nucleotides −90 to −67) and 3′-terminus (nucleotides 10,553 to 10,570) of the corresponding cDNA. The cloned cDNA sequence is contained in a genomic BAC clone (AC020716), which allowed localization of the gene encoding this putative PIKK family member to human chromosome 16. Based on its functional overlap with ATM, this new PIKK family member was named “ATX”.

The collective results of the 5′-RACE and RT-PCR assays of mRNA derived from Jurkat T cell, human brain, and other human cell lines indicated that the ATX locus gives rise to several mRNA transcripts (FIG. 1A). One repetitively isolated ATX cDNA clone contains exon 4 spliced directly to exon 6, and yields the 3,521 amino acid polypeptide described above. This mRNA transcript and encoded polypeptide has been designated “long ATX”, to distinguish it from a “short ATX” polypeptide (3,031 amino acids) produced as a result of allelic variation in exon 15, which leads to the insertion of 27 nucleotides beginning at nucleotide 1427 (FIG. 1A). This sequence alteration causes the insertion of two in-frame stop codons, and use of the next available ATG codon (in exon 16) as the translational start site gives rise to the amino-terminally truncated, short form of ATX. The 5′ end of the ATX allele that encodes short ATX is contained within a second genomic BAC clone (AC003007) derived from human chromosome 16. Yamashita et al. have identified two human cDNA clones, both designated hSMG-1, one of which (FIG. 1A, second from bottom, SEQ ID NO: 7) was similar to the long ATX cDNA clone (SEQ ID NO:1) (Yamashita et al., Genes and Development 15:2215-2228 (2001)). Exon 5 was not included in our long cDNA clone due to the infrequent appearance of this exon during our 5′-RACE and RT-PCR analyses of human cell line-derived mRNA. Furthermore, the minority of cDNAs that did include the exon 5 sequence frequently contained exon 3, which placed an in-frame stop codon at the 5′-end of this cDNA (FIG. 1A). The longest ATX cDNA clone (ORF beginning at exon 2, SEQ ID NO:9) identified by Yamashita et al. (Yamashita et al., supra, 2001) was also isolated in our screening procedure. However, it was repeatedly found that the exon 3-associated stop codon was present in this transcript.

### EXAMPLE 2

### Expression of Endoqenous and Recombinant ATX

In order to examine the expression of ATX mRNA in various tissues, a multiple tissue Northern blot was hybridized with a 32P-labelled, ATX cDNA probe that spanned exons 38-39(nucleotides 5,071-5,370). The ATX probe detected a major mRNA species that, based on its electrophoretic mobility, was significantly larger the 9.5 kb calibration marker, and could reasonably accommodate the predicted ORF (10.5 kb) of long ATX (data not shown). This ATX transcript was widely expressed in human tissues, with the highest levels observed in heart and skeletal muscle. These results are consistent with those obtained in immunoblot analyses with ATX-specific antibodies, which showed that ATX protein was uniformly expressed in hematopoietic, mesenchymal, and epithelial cell lines (data not shown). Database searches with the ATX amino acid sequence revealed the highest degree of homology to C. elegans SMG1, a protein required for NMD in the worm. Both ATX and CeSMG1 contain the PI 3-kinase related catalytic domain, which identifies these proteins as members of the PIKK family (FIG. 1B). Outside of the catalytic domain, the regional sequence homology between ATX and other PIKK family members was limited to the FKBP-12●rapamycin binding (FRB, designated NH2 in FIG. 1B) domain of mTOR, and to the NH1 and NH2 domains of CeSMG1. The FRB domain mediates the high-affinity interaction between mTOR and the antiproliferative FKBP12●rapamycin complex (Chen et al., Proc. Natl. Acad. Sci. USA, 92:4947-4951 (1995)). However, the FRB-related domain of ATX does not confer any detectable binding affinity for FKBP12●rapamycin (data not shown); hence, it is unlikely that ATX is a relevant target for rapamycin in intact cells. The expression of the short and long forms of ATX were compared after transient transfection of the respective cDNAs into human embryonic kidney 293T cells. The short ATX polypeptide was poorly expressed relative to long ATX (data not shown). However, these results do not exclude the possibility that the shorter form of ATX is expressed and contributes to the overall functions of ATX in mammalian cells.

In order to compare the translation product derived from the long ATX cDNA with the endogenously expressed ATX polypeptide, HEK 293T cells were transfected with HA-tagged expression plasmids encoding either wild-type ATX (HA-ATXWT) or a catalytically inactive ATX mutant (HA-ATXKI). The HA-ATXKI mutant contains an Asp>Ala substitution at a conserved residue (Asp-2195) in the ATX catalytic domain. Similar mutations in the catalytic domains of ATM, ATR, and DNA-PKCS have been shown to generate kinase-inactive (KI) versions of these PIKK family members (Canman et al., Science, 281:1677-1679 (1998); Cliby et al., EMBO Journal, 17:159-169 (1998); Hunter, supra, 1995).

For analyses of the endogenous ATX protein, two different rabbit polyclonal antibodies were prepared against GST fusion proteins containing peptide fragments derived from ATX. The first antibody (α-ATX Ab-1) was generated against a GST fusion protein containing amino acids 2281-2339 of ATX, while the second (α-ATX Ab-2) was obtained from immunizations with GST fused to amino acids 1691-1790 of ATX. The α-ATX Ab1 immunoblot analyses of whole cell extracts from non-transfected or HA-ATX-transfected HEK 293T cells revealed a single major immunoreactive band migrating, at the predicted molecular mass of ˜395 kDa (data not shown). An immunoreactive protein bearing a nearly identical electrophoretic mobility was detected in α-HA immunoprecipitates from transfected 293T cells. These results indicate that the molecular mass of the recombinant protein produced from the long ATX cDNA corresponds closely to that of the endogenous ATX protein. Consistent with the predicted size of ATX, the band recognized by the α-ATX antibodies migrated with a significantly lower electrophoretic mobility than either ATM (molecular mass, 370 kDa) or ATR (molecular mass, 305 kDa).

Methods:

Cloning

The longer ATX ORF was appended with an amino-terminal hemagglutinin (HA) epitope tag sequence (CYPYDVPDYASL), and was subsequently amplified as two partially overlapping fragments from Jurkat cDNA. The nucleotide at position 4,620 was mutated in each of the two PCR products to create a SacII site that could be utilized to ligate the two ATX fragments, which were inserted into the XhoI and NotI sites of pcDNA3.1 (Invitrogen) (HA-ATX). The mutation used to generate the SacII did not alter the ATX polypeptide sequence. The expression vector encoding the catalytically inactive ATX mutant (HA-ATXKI) contains an Ala substitution at Asp-2195, which was generated by site-directed mutagenesis with the QuickChange kit (Stratagene). All plasmid constructs were sequenced to insure the fidelity of the PCR and cloning procedures.

Cell Lines

U2OS osterosarcoma and human embryonic kidney (HEK) 293T cells were cultured in low-glucose Dulbecco's Modified Eagle's Medium (DMEM), supplemented with 10% fetal bovine serum. The ATM-deficient human fibroblast line, AT4BI, was maintained in DMEM/F-12 medium supplemented with 10% fetal bovine serum. Where indicated, cells were γ-irradiated with a 137Cs source or UV irradiated with a UV-B source (λmax, 305 nm).

Antibodies

ATX-specific antibodies were raised by immunizing rabbits (Cocalico Biologicals, Inc.) with the indicated glutathione S-transferase (GST) fusion protein. Anti-ATX Ab-1 was raised against a GST fusion protein containing ATX amino acids 2281-2339, and α-ATX Ab-2 was raised against a GST fusion protein containing ATX amino acids 1691-1790. For purification of α-ATX Ab-2, GST-reactive antibodies were first absorbed on GSH-agarose. The flow-through fraction was then affinity purified over Affi-Gel 15 (BIO-RAD) coupled to the GST-ATX1691-1790 fusion protein. The α-PLC-γ1 antiserum was prepared as described (Secrist et al., J. Biol. Chem., 268: 5886-5893 (1993)). The α-ATM (Ab-3), α-ATR (Ab-1), α-phospho-Ser15-p53, and α-p53 (Ab-6) reagents were obtained from Oncogene Science Research Products. Additional antibodies were obtained from (sources in parentheses): α-HA (clone 12CA5; BabCo), α-FLAG-M2 and α-tubulin (Sigma), α-Cds1/Chk2 (Upstate Biotechnology), and α-GAL4 (clone RK5C1; Santa Cruz Biotechnology).

Two-Dimensional Page

HEK 293 cells were lysed and protein analyzed as described {Pal, 2001 #1360}, except that cellular extracts were incubated for 2 h with α-FLAG-M2 mAb, followed by 2 h with protein G agarose (Sigma) to immunoprecipitate the FLAG-hUpf1 protein. Prior to elution, the immunoprecipitates were washed in lysis buffer as described {Pal et al.,Rna 7:5-15 (2001) #1360}.

Immunofluorescence

For immunofluorescence microscopy of endogenous ATX, 6×104 U2OS cells were plated onto 22-mm2 glass coverslips. After 40 h, cells were exposed to 400 J/m2 UV-B, then fixed 1, 4 or 8 hrs later in phosphate-buffered saline (PBS) containing 4% paraformaldehyde for 20 min, and incubated in methanol at −20° for 15 min. The coverslips were rehydrated in phosphate-buffered saline (PBS) and incubated overnight at 4° C. in blocking solution (PBS containing 3% BSA and 2% goat serum). Coverslips were subsequently overlayed for 1 h with affinity purified α-ATX Ab-2 (1 μg per ml) in blocking solution at room temperature. Coverslips were washed with PBS, 0.2% Tween-20, and overlayed for 45 min at room temperature with fluorescein isothiocyanate (FITC)—conjugated goat anti-rabbit IgG (Caltag) (1:2000 in blocking solution). Samples were then washed and incubated with 100 μg per ml RNaseA in PBS for 30 min, followed by 1 μg per ml propidium iodide for 5 min. After extensive washing in PBS containing 0.2% Tween-20, coverslips were mounted on slides with an aqueous anti-fade mounting reagent (Vectashield, Vector Laboratories). Immunofluorescence images were generated with a Carl Zeiss LSM410 scanning laser confocal microscope.

### EXAMPLE 3

### Characterization of ATX Kinase Activity

With the exception of the TOR proteins, the PIKK family members that bear functional catalytic domains phosphorylate substrates bearing the Ser/Thr-Gln motif (Tibbetts and Abraham, supra, 2000). To determine whether the ATX kinase domain displayed a similar phosphorylation site selectivity, HEK 293T cells were transfected with a plasmid vector encoding HA-tagged ATXWT, ATXKI, or, for comparative purposes, HA-ATMWT. Detergent extracts from the transfected cell populations were immunoprecipitated with α-HA antibody, and protein kinase assays were performed in buffer containing Mn2+, [γ-32P]ATP, and a GST fusion protein containing the first 70 amino acids of p53 (GST-p53₁₋₇₀) as the substrate (FIG. 1C). The GST-p53₁₋₇₀ protein was previously identified as a substrate for ATM and ATR in immune complex kinase assays (Tibbetts et al., Genes and Development 13:152-157 (1999)). Interestingly, the specific kinase activity of HA-ATXWT towards GST-p53₁₋₇₀ was significantly higher than that of HA-ATM (FIG. 1C, left panel). Based on the ratios of 32P incorporation into substrate to levels of HA-tagged protein kinase, it can be estimated that the specific kinase activity of ATXWT was approximately 3.5-fold higher than that of ATMWT. As observed with ATM as the test kinase (Banin et al., Science 281:1674-1677 (1998); Canman et al., Science 281:1677-1679 (1998); Tibbetts et al., supra, 1999), phosphorylation of GST-p53₁₋₇₀ by ATXWT was nearly abolished by substitution of the Ser-15 residue in p53 with Ala (FIG. 1C, right panel). Because Ser-15 resides in the optimal sequence (LSQE) for phosphorylation by ATM (O'Neill et al., J. Biol. Chem. 275:22719-22727 (2000)), this finding indicates that ATX is a Ser/Thr-Gln-directed kinase, with the potential to phosphorylate a set of substrates that overlaps with those modified by ATM. In contrast to the amino-terminal fragment, of p53, the PHAS-I/4E-BP1 protein, which is the prototypical substrate for mTOR, was poorly phosphorylated by HA-ATXWT in immune complex kinase assays (data not shown).

The amino acid sequences surrounding the four phosphorylation sites (LSQP, LSQD, LSQD, and LSQY) identified in this study resemble the consensus site for phosphorylation by ATM (O'Neill et al., supra, 2000). A GST fusion protein that contained the carboxyl-terminal region of hUpf1 (amino acids 1019-1118), including all four of the ATX phosphorylation sites was constructed. This GST-hUpf11019-1118 protein was tested as a substrate for HA-ATXWT versus HA-ATMWT in immune complex kinase assays. Once again, this substrate was phosphorylated by both ATM and ATX, with the latter protein kinase showing the higher specific catalytic activity under these in vitro assay conditions (FIG. 1C, left panel). Furthermore, the results of repeated assays indicated that GST-p53₁₋₇₀ was more avidly phosphorylated by ATX than was the GST-hUpf11019-1118 substrate.

The protein kinase activities of the mammalian PIKKs characteristically display a strong dependence on Mn2+ as a cofactor for the phosphotransferase reaction, and variable sensitivity to inhibition by wortmannin (Abraham, Genes and Development 15:2177-2196 (2001)). In our studies, the protein kinase activity of ATX was also dependent on the addition of Mn2+ the kinase reaction buffer (data not shown). In addition, pretreatment of the immunoprecipitated HA-ATXWT protein with wortmannin resulted in a concentration-dependent inhibition of GST-p53₁₋₇₀ phosphorylation. The drug concentration required for 50% inhibition (IC50) of ATX activity in vitro was between 10 and 100 nM (FIG. 1D), which is comparable to the previously published IC50 (80 nM) for wortmannin as an ATM inhibitor (Sarkaria et al., Cancer Res. 58:4375-4382 (1998)). Wortmannin is an irreversible inhibitor of PIKKs (Walker et al., Molecular Cells 6:909-919 (2000)) and can be used to assess the potency of this drug as an ATX inhibitor in intact cells. To this end, U2OS osteosarcoma cells were pretreated for 30 min with the indicated concentrations of wortmannin, followed by the preparation of cellular extracts for immunoprecipitation of endogenous ATX with α-ATX Ab-2. Under these conditions, wortmannin inhibited ATX kinase activity with an IC50 of 1-3 μM, which is considerably higher than that observed following direct treatment of the immunoprecipitated protein kinase with this drug (FIG. 1D). A similarly dramatic decrease in the inhibitory potency of wortmannin was observed with ATM as the target enzyme in intact cells (Sarkaria et al., supra, 1998).

Immune Complex Kinase Assays

Native or recombinant ATX proteins were immunoprecipitated from cell extracts as described above, and the immunoprecipitates were washed twice in lysis buffer, once in high-salt buffer (100 mM Tris-HCl, pH 7.4, 500 mM LiCl) and once in kinase buffer (10 mM Hepes, pH 7.4, 50 mM NaCl, 50 mM β-glycerol phosphate). Forty μl kinase buffer (containing 10% glycerol, 1 mM DTT, 10 mM MnCl₂, 20 nM microcystin, protease inhibitors, 1 μg of the indicated substrate, 10 μM ATP, and 10 μCi [γ-32P]ATP (6000 Ci/mmole) (NEN)] was added to each sample, and kinase reactions were performed for 30 min at 30° C. Reactions were terminated by addition of 40 μl of 4×-SDS-PAGE sample buffer, and heating to 100□C.

### EXAMPLE4

### Subcellular Localization of ATX

The subcellular localization of ATX was examined by biochemical fractionation of U2OS cells, followed by immunoprecipitation of crude nuclear and cytoplasmic fractions with α-ATX Ab-2. Comparable levels of ATX were found in the nuclear and cytoplasmic extracts from U2OS cells (data not shown). The integrity of these subcellular fractions was confirmed by immunoprecipitation and immunoblotting of parallel samples with antibodies specific for PLCγ1 and ATR, which are localized to the cytoplasm and nucleus, respectively. The presence of ATX in both the cytoplasmic and nuclear compartments was further documented by immunostaining of U2OS cells with affinity-purified α-ATX Ab-2. Exposure of cells to genotoxic agents triggers the appearance of DNA damage-induced nuclear foci containing either ATM or ATR (Andegeko et al., J. Biol. Chem. 276:38334-38230 (2001); Tibbetts et al., Genes and Development 14:2989-3002 (2000)). To determine whether ATX undergoes similar changes in subcellular localization in response to genotoxic stress, U2OS cells were treated with 400 J/m2 UV-B, and stained with α-ATX Ab-2. Exposure to UV triggered the appearance of ATX-containing nuclear foci. The ATX-positive foci were evident within 1 h after UV treatment, and continued to accumulate in the cells until at least 8 h post-treatment, at which time greater than 50% of the cells exhibited multiple ATX-containing foci. In contrast, the formation of ATX foci after treatment of U2OS cells with 20 Gy IR was not detected.

In addition, the effect of genotoxic stress on the protein kinase activity of ATX in immune complex assays was determined. Consistent with the results of the immunofluorescence staining experiments, treatment of the cells with IR failed to induce a reproducible increase in the protein kinase activity observed in α-ATX immunoprecipitates (data not shown). On the other hand, UV exposure caused a modest but consistent increase in ATX kinase activity that reached a maximal level at 4 h post-irradiation. Collectively, the results of the nuclear localization and protein kinase activity studies indicated that, like ATM and ATR, ATX participated in cellular responses to DNA damage or other forms of stress induced by UV irradiation.

Methods:

Cell Fractionation, Immunoprecipitation, and Immunoblotting

For subcellular fractionations, U2OS cells were resuspended in cold homogenization buffer (25 mM Hepes, pH 7.4, 250 mM sucrose, 1 mM EGTA, 5 mM MgCl2, 50 mM NaF, 1 mM DTT, plus protease inhibitors) and Dounce homogenized on ice with 40 strokes in a Tefloncoated homogenizer. The nuclei were pelleted at 500×g, and the supernatant was collected as the crude cytoplasmic fraction. Prior to immunoprecipitation, 150 mM NaCl and 1% (wt/vol) NP-40 (final concentration) were added to the crude cytoplasmic fractions. Nuclear extracts were prepared by suspending the nuclear pellets in extraction buffer (50 mM Tris-HCl, pH 7.4, 150 mM NaCl, 1% NP-40, 1 mM dithiothreitol), supplemented with protease inhibitors (10 μg per ml leupeptin, 10 μg per ml aprotinin, 1 μM pepstatin). After 15 min on ice, the samples were centrifuged, and the supernatant was collected for analysis. For immunoprecipitations, cell extracts were prepared by resuspending washed cell pellets in lysis buffer (50 mM Tris-HCl, pH 7.4, 150 mM NaCl, 1% NP-40, 1 mM dithiothreitol) plus protease inhibitors. When samples were prepared for immune complex kinase assays, the lysis buffer was modified to contain 1% Tween-20 detergent in place of NP-40, and additional phosphatase inhibitors (20 mM β-glycerol phosphate and 50 nM microcystin). Samples were incubated on ice for 15 min, and then clarified by centrifugation. HA-tagged recombinant proteins were immunoprecipitated from cell extracts with 4 μg of α-HA antibody. Endogenous ATX protein was immunoprecipitated with 8 μg of α-ATX Ab-2. After separation by SDS-PAGE, the proteins were detected by autoradiography (for kinase reaction products) or by immunoblotting. Proteins immunoblotted with rabbit and mouse antibodies were detected with protein A-horseradish peroxidase (HRP) (Amersham), and sheep anti-mouse IgG-HRP (Amersham), respectively. Immunoreactive proteins were illuminated with Renaissance chemiluminescence system (NEN).

### EXAMPLE 5

### Effect of Decreased ATX Function on Cellular Sensitivity to UV and IR

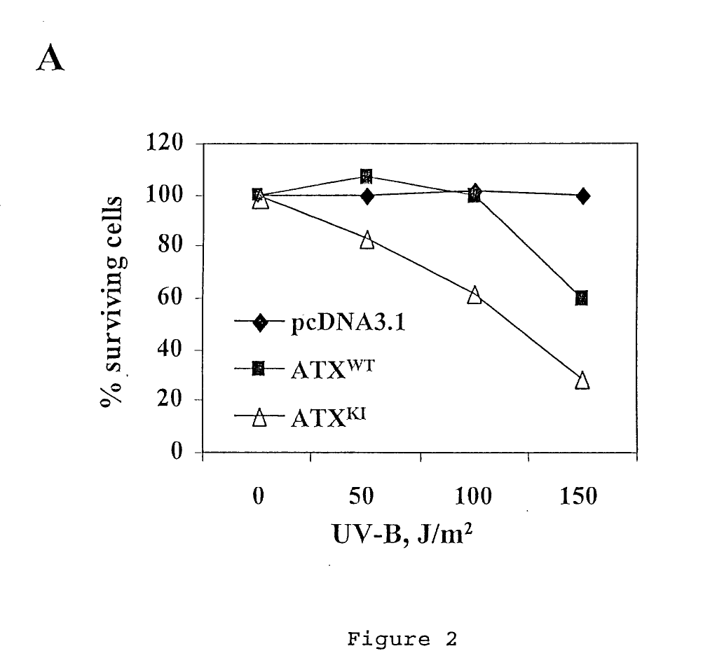

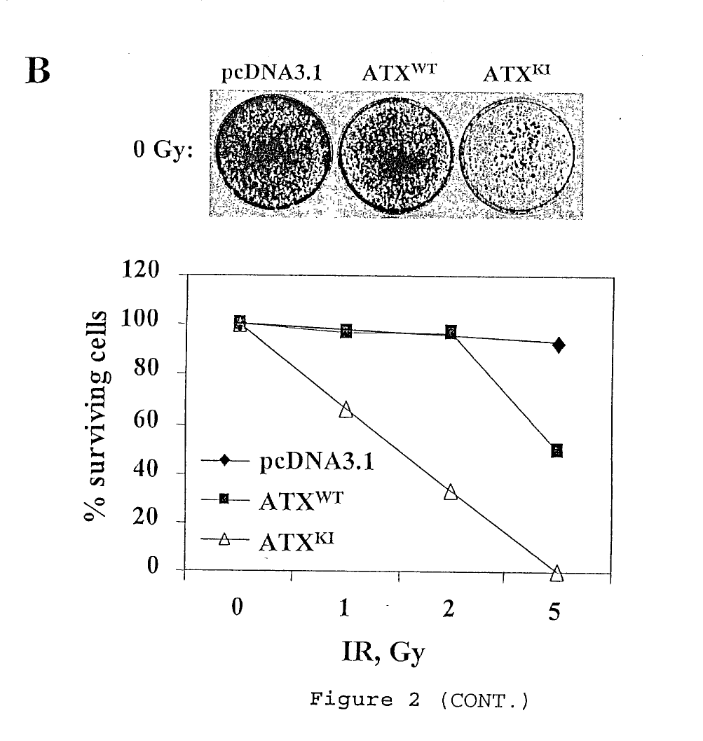

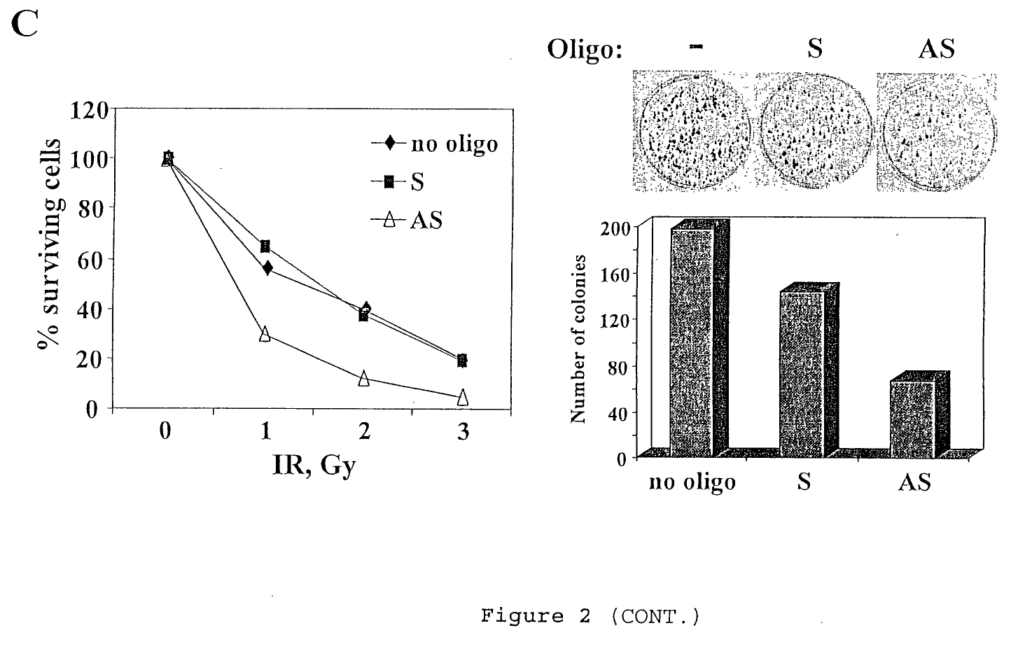

In order to gain further insights into the role of ATX in stress responses, U2OS cells were transfected with the kinase-inactive ATXKI mutant, and the UV- and IR-sensitivities of the transfected cells in clonogenic survival assays was examined. Control cell populations were transfected with either empty plasmid (pcDNA3.1) or with ATXWT-encoding plasmid. At 48 h post-transfection, the cells were treated with various doses of UV-B (FIG. 2A) or IR (FIG. 2B). The treated cells were then cultured in G418-containing medium in order to select for stably transfected cells. Cellular survival was determined after 10 days in culture by staining emergent colonies with Coomassie blue, followed by calorimetric quantitation of the amount of dye-bound protein present in each sample. Expression of ATXKI, but not ATXWT, reduced the basal survival of otherwise untreated U2OS cells (FIG. 2B, top panel). These results indicate that endogenous ATX function is required for the maintenance of normal cell viability or growth in culture. Furthermore, expression of ATXKI dramatically increased the sensitivity of U2OS cells to the cytostatic/cytotoxic effects of both UV and IR in these clonogenic survival assays.

To address concerns related to potential non-specific effects of ATXKI expression on cellular functions, an antisense oligonucleotide-based approach to reduce the level of ATX expression in U2OS cells was developed. The cells were mock transfected, or transfected with sense (S) or antisense (AS) oligonucleotides, and then treated for 24 h with various genotoxic agents. The cells were then trypsinized and replated, and clonogenic survival was analyzed after 10 days in culture. The results obtained with AS-treated cells were strikingly similar to those obtained with the ATXKI-expressing cells (FIGS. 2A-C). While transfection of U2OS cells with the S oligonucleotide reduced the basal level of colony outgrowth by 25%, treatment with the AS oligonucleotide decreased basal clonogenic activity by 75% (FIG. 2C, right panel). Hence, the AS-induced decrease in ATX protein expression was accompanied by a reduction in cell viability or proliferation in the absence of environmental stress. Furthermore, the AS-treated cells were significantly more sensitive to the suppressive effects of IR (FIG. 2C, left panel) and UV treatments (data not shown) on clonogenic survival. The reduction in ATX protein caused by AS treatment ranged from 50-90% in over 10 independent trials.

Methods:

Cell Transfections

To prepare recombinant HA-tagged ATX and ATM proteins, HEK 293T cells were plated onto 60-mm dishes (9×105 cells per dish), and were transfected with 5 μg pcDNA3.1 (empty vector), HA-ATXWT, HA-ATXKI, or HA-ATMWT plasmid DNAS. Transfections were performed with the Fugene 6 transfection reagent (Roche), according to the manufacturer's instructions. For NMD assays (see Example 9, below), U2OS cells were seeded onto 100 mm dishes (1×106 cells per dish). After 20 h, cells were transfected with 1.5 μg of pmCMV-Gl test plasmid, either Norm or 39Ter (Ishigaki et al., 2001); 1.5 μg of phCMV-MUP reference plasmid (Ishigaki et al., Cell 106:607-617 (2001)); and 7 μg of empty plasmid (pcDNA 3.1), plasmids encoding HA-ATMWT, HA-ATMKI, HA-ATXWT or HA-ATXKI. For antisense transfection experiments, U2OS cells were seeded onto 60 mm dishes (1×10⁵ cells per dish) in complete medium supplemented with penicillin/streptomycin. After 30 h, cells were either mock transfected or transfected with sense (S) or antisense (AS) phosphorothioate oligonucleotides (Genset Oligos, La Jolla, Calif.). The S oligonucleotide spans ATX nucleotides 210-229 (5′-GAGACAGGAGGGAGCTTGCT-3′), and the AS oligonucleotide is complementary to this sequence (5′-AGCAAGCTCCCTCCTGTCTC-3′). The cells were transfected with oligonucleotides at final concentrations of 8 μg/ml, with Fugene 6:DNA ratio of 1.6:1. Forty-eight hours after transfection, dishes were exposed to IR, UV-B, or 5-FU, and then harvested for immunoblotting, cell-cycle distribution, or cell survival assays. To examine ATX protein levels in oligonucleotide-treated cells, whole cell extracts were resolved by SDS-PAGE and immunoblotted with α-ATX Ab-1. When oligonucleotide-transfected cells were used for NMD assays, U2OS cells were seeded in culture dishes as described above. The cells were transfected using the Fugene reagent, with 1.5 μg of pmCMV-Gl test plasmid, 0.7 μg of phCMV-MUP reference plasmid, and 24 μg of S or AS oligonucleotide.

Clonogenic and G418 Survival Assays

U2OS cells were seeded into 60 mm dishes (1×105 cells per dish) in complete medium. After 48 h, cells were transfected as described above. Forty-eight hours after transfection, dishes were exposed to IR or UV-B, and G418 was added at 1 mg per ml in complete medium. G418-resistant cells were stained 10 days later with Coomassie Blue. To quantitate the outgrowth of drug-resistant cells, the Coomassie Blue-bound protein was solubilized at 37° C. with 0.1 M NaOH, and the soluble material was analyzed by absorbance spectroscopy at a wavelength of 590 nm. For AT4BI cell survival assays, cells were transfected with pcDNA3.1, pcDNA3.1-FLAG-ATM, or HA-ATX. After 48 h, the transfected cells were exposed to the indicated doses of IR, and G418 was added at 8 hours post-irradiation. Drug-resistant colonies were stained with Coomassie Blue after 10 days in culture, and the samples were analyzed with Image Pro Plus software to quantitate cell density. For clonogenic assays where oligonucleotides were used, the cells were plated and transfected with S or AS oligonucleotides as described above. Forty-eight hours after transfection, cells were exposed to IR or UV. Twenty-four hours after exposure to damaging agents, cells were replated at 1000 cells per 60 mm dish and colonies allowed to form for 10 days. Dishes were stained with Coomassie Blue, and the number of colonies (minimum size, 50 cells per colony) was counted by visual examination.

### EXAMPLE 6

### Role of ATX in p53 Activation

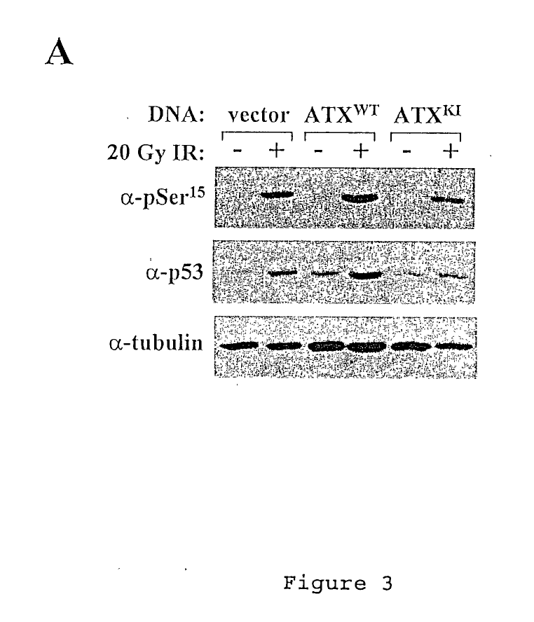

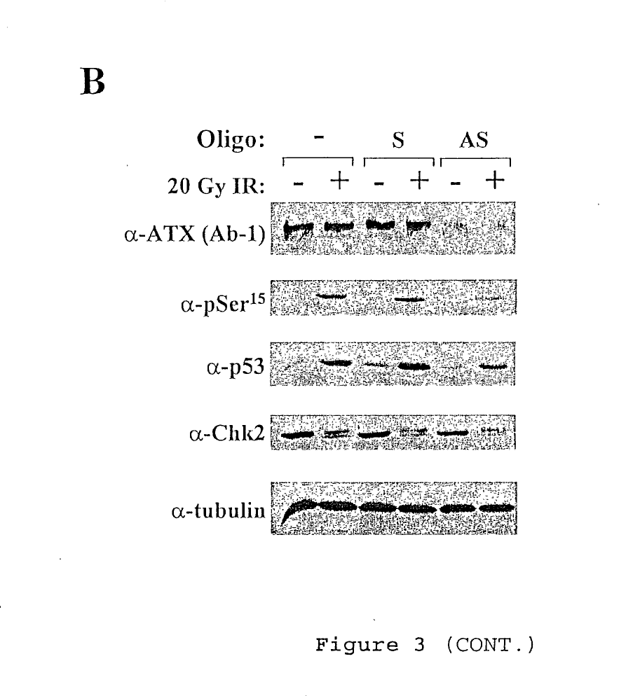

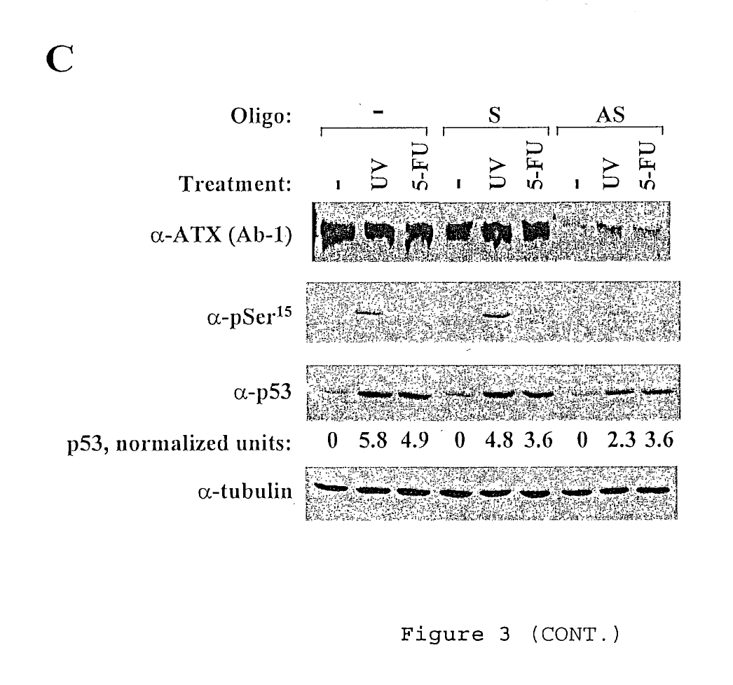

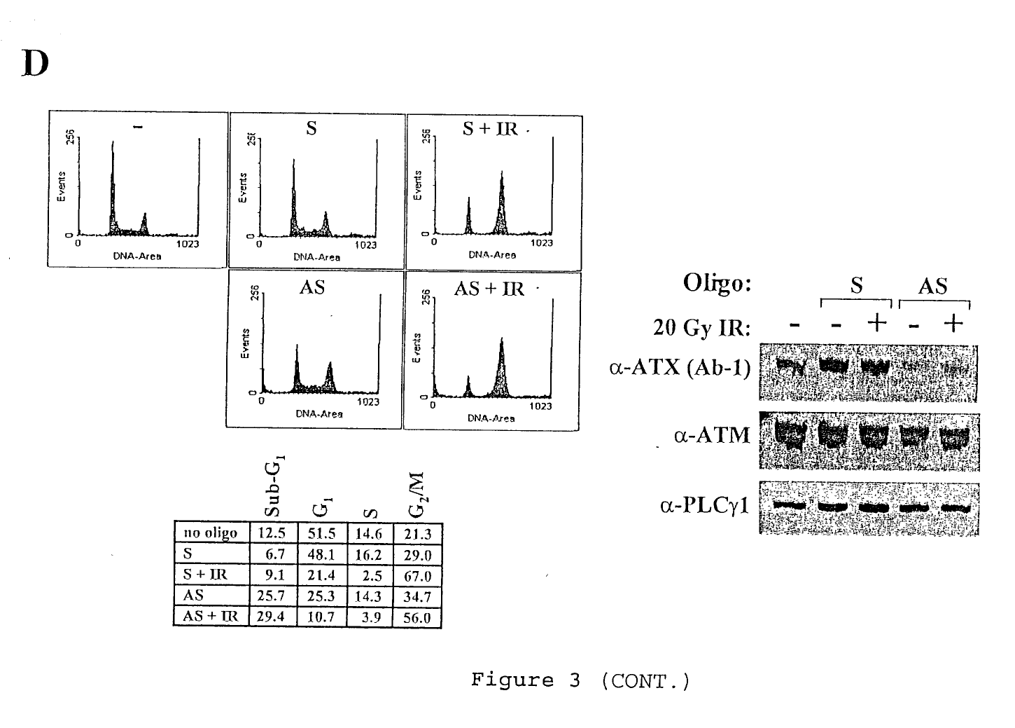

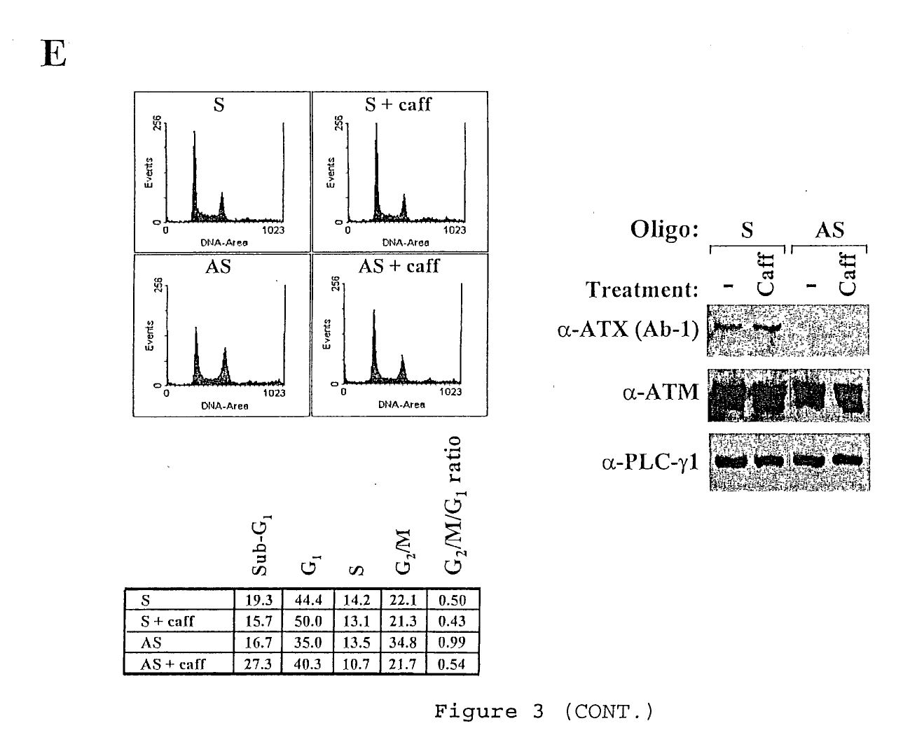

A major mediator of stress-induced signaling in mammalian cells is the tumor suppressor protein, p53 (Ko and Prives, Genes and Development 10:1054-1072, (1996); Ryan et al., Curr. Opin. Cell Biol. 13:332-337 (2001)). ATX phosphorylates p53 on Ser-15 (FIG. 1C), a site implicated in the regulation of p53 stability and transcriptional activity (Dumaz and Meek, Curr. Opin. Cell Biol. 13:225-231 (1999); Zhang and Xiong, Science 292:1910-1915 (2001)). Therefore, the possibility that these two proteins are functionally linked during cellular stress responses was investigated. U2OS cells were transiently transfected with a HA-ATXWT or HA-ATXKI expression plasmid, together with a GFP-encoding plasmid to allow for FACS-based enrichment of the transfected cells. The GFP+ cells were then examined for IR-induced stabilization of p53, as well as for specific phosphorylation of this protein on Ser-15. Expression of ATXKI strongly suppressed both the phosphorylation of Ser-15 and the overall accumulation of p53 in IR-treated cells (FIG. 3A). In contrast, overexpression of ATXWT enhanced both of these responses in cells exposed to IR. Consistent with findings presented above, treatment of U2OS cells with the AS oligonucleotide led to a decrease in endogenous ATX expression, and concomitantly reduced both the phosphorylation and stabilization of p53 induced by IR exposure (FIG. 3B). These results indicated that ATX exhibits functional overlap with ATM during IR-induced p53 activation.

Recent findings point toward ATM as a critical upstream regulator of the activity of the checkpoint kinase, hChk2, in IR-damaged cells (Ahn et al., Cancer Res. 60:5934-5936 (2000); Melchionna et al., Nat. Cell Biol. 2:762-765 (2000)). To determine whether ATX was also involved in hChk2 activation, the effect of AS oligonucleotide treatment on the IR-dependent phosphorylation of hChk2 was examined. In contrast to the p53 results, the AS-treated cells retained the ability to phosphorylate hChk2 in response to IR-induced stress (FIG. 3B). These results indicate that, while ATM and ATX serve as positive regulators of p53 function, ATX plays no identifiable role as an upstream activator of a distinct ATM target protein, the hChk2 kinase. Moreover, the differential effects of AS treatment on p53 expression versus hChk2 activation argue against the possibility that AS exposure leads to nonspecific inhibition of checkpoint signaling responses to IR-induced DNA damage.

Additional studies with AS oligonucleotide-treated cells demonstrated that, in contrast to ATM (Canman et al., Science 281, 1677-1679. 1998; Siliciano et al., Genes Dev 11, 3471-3481 (1997)), ATX plays a role in the phosphorylation and stabilization of p53 in cells exposed to UV light (FIG. 3C). As observed with IR as the stress-inducing agent, a reduction in ATX protein expression severely impaired both Ser-15 phosphorylation and p53 protein accumulation in UV-damaged cells. The recognition of UV light-induced DNA damage occurs primarily during S phase, when pyrimidine dimers and other bulky lesions interfere with replication fork progression (Friedberg, DNA Repair and Mutagenesis (Washington, D.C., ASM Press) (1995); Lindahl and Wood, Cell 103:1121-1131 (1999)).

In order to further define the potential linkage between DNA replicational stress and ATX, the response of AS-treated cells to 5-fluorouracil (5-FU), an S-phase specific cytotoxic agent was examined (Danenberg et al., Seminars in Oncology 26:621-631 (1999); Grem, Investigational New Drugs 18:299-313 (2000)). Previous findings indicated that the cytotoxic effects of 5-FU are strongly p53-dependent (Bunz et al., J. Clin. Invest. 104:263-269 (1999)). Treatment of U2OS cells with 5-FU increased p53 expression to levels similar to those observed in UV-irradiated cells (FIG. 3C). However, the accumulation of p53 induced by 5-FU exposure was not accompanied by an increase in Ser-15 phosphorylation. These findings indicate that the mechanism of p53 stabilization triggered by 5-FU does not involve upstream protein kinases that modify the Ser-15 site. Consistent with this conclusion, the level of p53 induction by 5-FU in AS-treated cells was identical to that observed in their S-treated counterparts. These results indicate that the inhibitory effect of the AS treatment on p53 activation is selective for those forms of stress that induce the phosphorylation of p53 at Ser-15.

Changes in phosphorylation at Ser-15 are typically accompanied by alterations in the expression of the p53 protein, which complicates the interpretation of results obtained by immunoblotting of whole cell extracts with phospho-Ser-15-specific antibodies. In order to confirm that reduced ATX expression interferes with stress-induced Ser-15 phosphorylation, U2OS cells were transfected with either the S or AS oligonucleotide, and then were pretreated with the proteasome inhibitor, LLnV, to stabilize p53. In the presence of LLnV, the p53 level in each test population was relatively unaffected by UV exposure (data not shown). However, the ratio of phospho-Ser-15 to total p53 protein was increased by UV irradiation of both the mock-transfected and S oligonucleotide-treated cells. Although AS treatment partially interfered with the accumulation of p53 under these conditions, the reduction in ATX expression effectively blocked the stoichiometric increase in Ser-15 phosphorylation triggered by UV-induced stress.

### EXAMPLE 7

### Role of ATX in IR-Induced Cell Cycle Arrest

Since p53 plays a central role in activation of the G1 checkpoint, and influences S, G2, and M checkpoints as well (Giaccia and Kastan, Genes & Development 12:2973-2983 (1998); Ko and Prives, supra, 1996), a functional deficiency of ATX might alter the cell-cycle arrest responses to IR and other genotoxic agents. To test this possibility, U2OS cells were pre-treated with S or AS oligonucleotides, exposed to IR, and cell-cycle distributions at 24 h post-irradiation were determined. In the absence of IR, AS treatment led to a reduction in the percentage of G1 phase cells, and a concomitant accumulation of G2/M phase cells, when compared to their S oligonucleotide-treated counterparts (FIG. 3D). The AS-treated cells also contained an increased subpopulation with <2N DNA content, which is indicative of apoptotic cells. After IR exposure, cells treated with the S oligonucleotide accumulated in both G1 and G2/M phases and were cleared out of S phase, a profile typical of p53-positive cells that retain G1 checkpoint function. In contrast, the AS-treated cells arrested primarily in G2/M phase after IR exposure. The cell-cycle distribution of the AS-treated cells was reminiscent of that observed in cells that have lost p53-dependent checkpoint function (North and Hainaut, Pathol. Biol. 48:255-270 (2000); Waldman et al., Cancer Res. 55:5187-5190 (1995)). Immunoblot analyses of the same cell populations confirmed that AS exposure led to a profound reduction in ATX protein levels in U2OS cells. In contrast, AS exposure caused no significant change in the expression levels of two control proteins, ATM and PLC-γ1.

The cell-cycle distribution results described in FIG. 3D demonstrated that ATX-deficient cells accumulate with 4N DNA content under both basal culture conditions and after IR-induced stress. This arrest state could reflect the activation of either a G2 or a mitotic checkpoint (or both checkpoints). To distinguish between these possibilities, the effects of caffeine, a known inhibitor of the G2 checkpoint (Powell et al., Cancer Res. 55:1643-1648 (1995); Russell et al., Cancer Res. 55:1639-1642 (1995); Yao et al., Nat, Med. 2:1140-1143 (1996)), on the cell cycle distribution of the AS-treated cells were examined. The G2 checkpoint inhibitor was added to the culture medium at 8 h prior to harvest for determination of cell-cycle distributions (FIG. 3E, left panel), and immunoblotting for ATX expression (right panel). Caffeine completely reversed the accumulation of G2/M phase cells induced by AS treatment, indicating that ATX deficiency triggered the activation of a caffeine-sensitive G2 checkpoint. The immunoblotting results confirmed that AS-treated cells displayed a marked, specific reduction in ATX protein expression. In addition, treatment of the AS cells with caffeine also resulted in an increase in the percentage of hypodiploid cells, which indicates that an intact G2 checkpoint partially protects the ATX-deficient cells from apoptotic death, for example, by preventing a catastrophic entry into M phase.

### EXAMPLE 8

### ATX Overexpression Complements IR Sensitivity in ATM-Deficient Cells

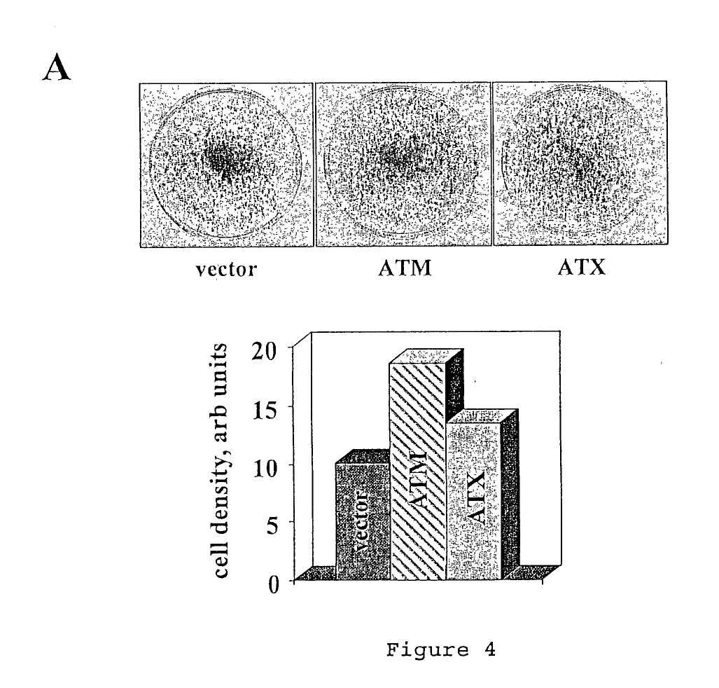

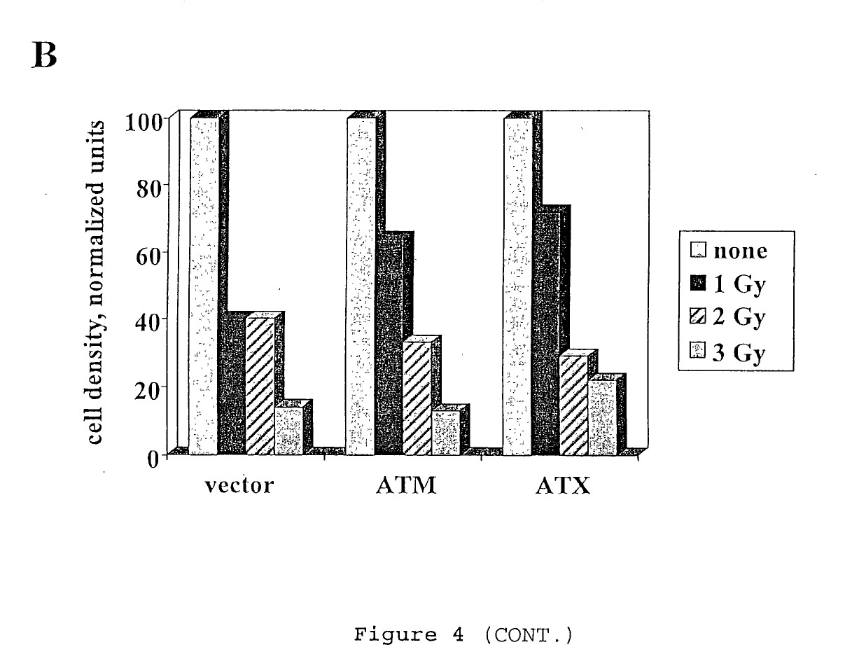

Based on the finding that ATM and ATX display overlapping functions as activators of p53, it was investigated whether ATX overexpression can complement the phenotypic defects found in cells from A-T patients. One characteristic defect of cells from A-T patients is reduced clonogenic survival in culture, even in the absence of DNA dsb-inducing agents (Rotman and Shiloh, Oncogene 18:6135-6144 (1998)). As shown in FIG. 4A, transient transfection of ATM-null AT4BI cells with an ATM expression plasmid increased the outgrowth of G418-resistant colonies by approximately 2-fold, relative to cells transfected with empty vector. The clonogenic defect of AT4BI cells was partially rescued (approximately 1.5-fold increase in colony survival) by transient expression of ATX. Thus, ATX overexpression partially complements the intrinsic clonogenic survival defect of ATM-null cells. Furthermore, low-dose (1 Gy) IR treatment sharply reduced the clonogenic survival of mock-transfected AT4BI cells, and this radiosensitive phenotype was rescued to equivalent degrees by transfection of the cells with ATM or ATX (FIG. 4B). Thus, overexpression of ATX complements, at least in part, the stress response defects observed in cells from A-T patients.

### EXAMPLE 9

### Roles of ATX and ATM in hUpf1 Phosphorylation and NMD

The Upf1 helicase undergoes serum-inducible phosphorylation in intact cells, as demonstrated by two-dimensional(2-D) gel electrophoresis (Pal et al., supra, 2001). Based on the evidence disclosed herein that ATX is a UV-responsive kinase, the possibility that UV light exposure triggers the phosphorylation of hUpf1 in ATXWT-transfected U2OS cells was investigated. Serum stimulation or UV treatment induced virtually identical shifts in the 2-D electrophoretic mobility of hUpf1, which indicates that these agents provoke the phosphorylation of this protein at similar sites (data not shown). In contrast, expression of the catalytically-inactive ATXKI protein blocked the appearance of the most highly shifted form of hUpf1, and caused the accumulation of a broad band with intermediate electrophoretic mobility. The latter hUpf1 species can be less phosphorylated forms of the protein. Thus, overexpression of ATXKI interferes with both the serum- and UV-induced phosphorylation of hUpf1; however, these results also indicate that the hUpf1 is targeted by at least one additional protein kinase in these cells.

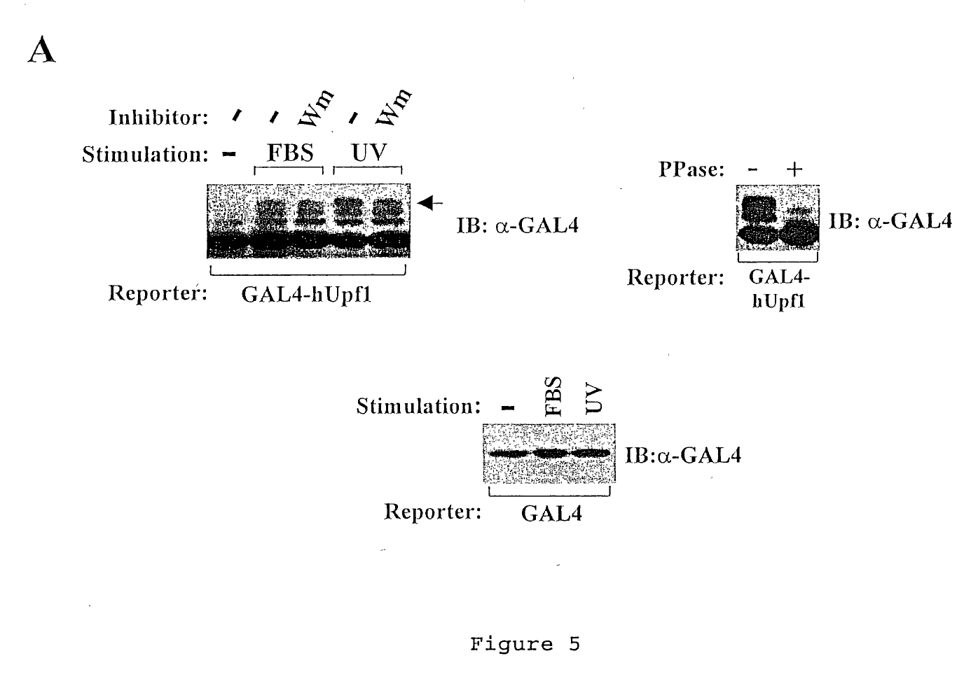

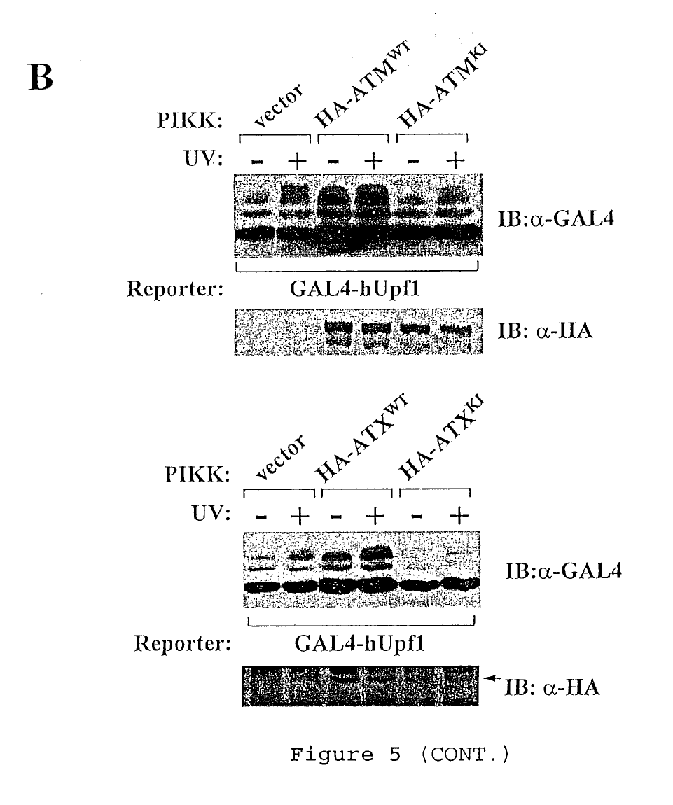

Based on the functional overlap between ATM and ATX during stress-induced p53 activation, it was possible that these PIKKs might also share the ability to regulate the RNA surveillance pathway leading to NMD. To focus our studies of hUpf1 phosphorylation on the Ser-Gln-rich region, a mammalian expression vector encoding GAL4 fused to the carboxyl terminus (amino acids 1019-1118) of hUpf1 (GAL4-hUpf11019-1118) was generated. Expression of GAL4-hUpf11019-1118 in U2OS cells generates a major immunoreactive band that migrates with a molecular mass of ˜35 kDa in serum-starved cells. Stimulation of the transfected cells with 10% fetal bovine serum or UV light leads to the increased expression of forms of GAL4-hUpf11019-1118 that display reduced electrophoretic mobility (FIG. 5A, left panel). In this experiment, cells were harvested at 6 h after serum or UV exposure; however, the GAL4-hUpf11019-1118 mobility shifts could be detected as early as 2 h after cellular stimulation with either agent. The appearance of these shifted GAL4-hUpf11019-1118 bands is due to phosphorylation, as treatment of the cell extracts with λ phosphatase collapses the complex pattern of α-Gal4-reactive species into the major ˜35 kDa band, which represents non-phosphorylated GAL4-hUpf11019-1118 (FIG. 5A, right panel). Moreover, the observed GAL4-hUpf11019-1118 mobility shifts were due to phosphorylation of the hUpf1 fragment, as the electrophoretic mobility GAL4 alone was not altered by cellular exposure to serum or UV (FIG. 5A, lower panel). In the experiment shown in FIG. 5A (left panel), selected samples were pretreated with 20 μM wortmannin in order to inhibit endogenous ATX and ATM kinase activities (Sarkaria et al., supra, 1998)). In the wortmannin-treated cells, the FBS- or UV-induced generation of the most slowly migrating form of GAL4-hUpf11019-1118 (indicated with an arrow) was preferentially inhibited. This drug effect was accompanied by an increase in the abundance of the less shifted bands, which can represent less phosphorylated forms of GAL4-hUpf11019-1118. These results indicate that, although a wortmannin-sensitive protein kinase(s) contributes to the inducible phosphorylation of the GAL4-hUpf11019-1118 reporter protein, the hUpf1 carboxyl-terminal region is also targeted for modification by at least one additional, wortmannin-resistant protein kinase.

To further examine the contributions of ATM and ATX to the phosphorylation of GAL4-hUpf11019-1118, U2OS cells were cotransfected with wild-type (WT) or kinase-inactive (KI) versions of HA-ATM or HA-ATX. Expression of either HA-ATMKI or HA-ATXKI strongly suppressed the phosphorylation of GAL4-hUpf11019-1118 in cells treated with UV light (FIG. 5B) or serum (data not shown). Expression of the catalytically active HA-ATMWT or HA-ATXWT proteins enhanced the phosphorylation of GAL4-hUpf11019-1118 in both unstimulated and stimulated cells. The latter results add further support to the notion that ATX and ATM are capable of phosphorylating hUpf1 carboxyl-terminal region in intact cells. In these experiments, the HA-tagged ATM and ATX proteins were overexpressed by approximately 2- and 1.5-fold, respectively, when compared to their endogenous counterparts (data not shown).

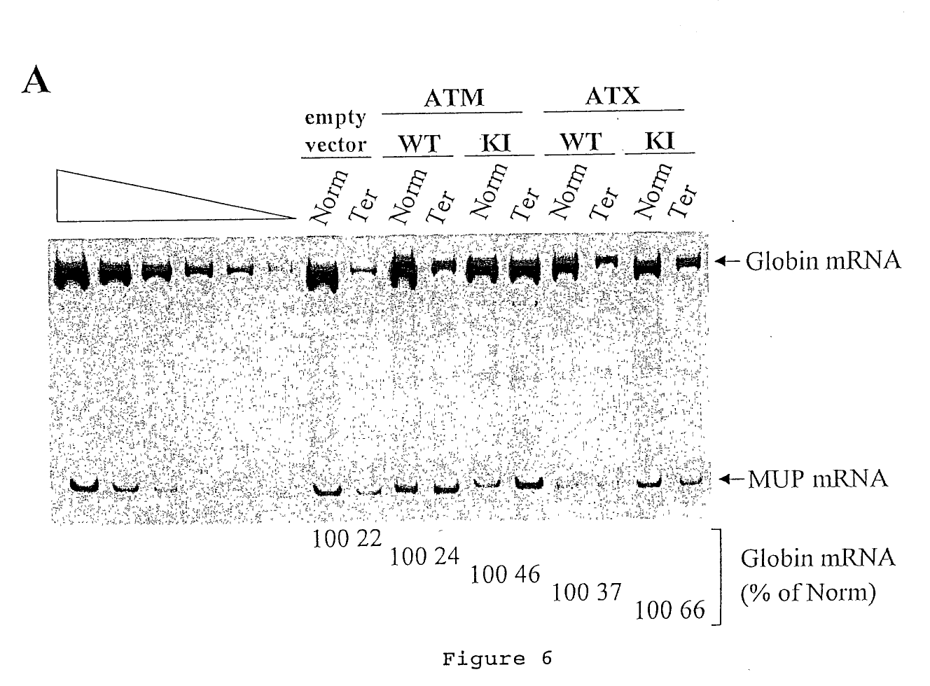

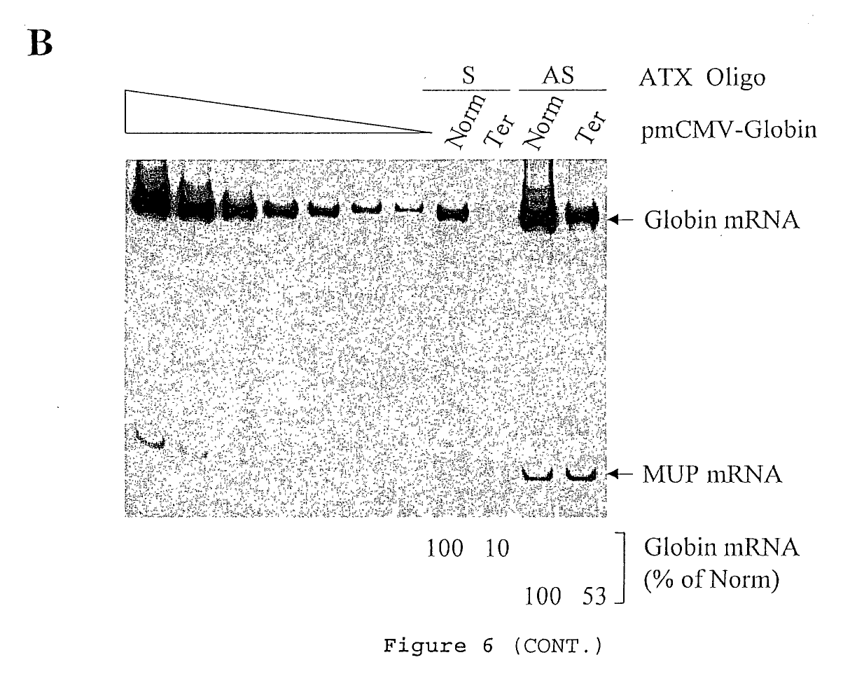

In addition, the effects of HA-ATXKI and HA-ATMKI overexpression on NMD were comparatively examined using an established assay (Sun et al., Proc. Natl. Acad. Sci. USA 95:10009-1998). U2OS cells were transfected with a plasmid encoding either the normal human β-globin gene (Norm) or a mutated β-globin gene bearing a premature termination codon (Ter), together with a reference plasmid encoding the mouse urinary protein (MUP). Where indicated, the cells were co-transfected with empty vector, or expression vectors encoding wild-type or kinase-inactive versions of ATM (HA-ATMWT, HA-ATMKI) or ATX (HA-ATXWT, HA-ATXKI) (FIG. 6A). Expression of the kinase-inactive HA-ATMKI or HA-ATXKI proteins abrogated NMD of the Ter-containing β-globin mRNA. Furthermore, treatment of the cells with the AS oligonucleotide to reduce endogenous ATX expression confirmed that ATX expression is required for maximal NMD activity under these assay conditions (FIG. 6B). Collectively, these results indicate that ATM and ATX function as shared components of the pathways leading to both NMD and p53 activation during UV- and IR-induced stress.

Methods:

Construction of GST and GAL4-hUpf1 Fusion Proteins

The hUpf11019-1118-BamHI fragment was generated by PCR amplification of full-length hUpf1 using the following primers: 5′-AGGAGGGGATCCGGACGCCAGAAGAACCGCTTTGGG-3′, 5′-AGGAGGGGATCCATACTGGGACAGCCCCGTCAC-3′. This fragment was subcloned into the BamHI site of pGEX-2T and pCMX-GAL4(N) to generate the GSThUpf11019-1118 and GAL4-hUpf11019-1118 fusion proteins, respectively.

GAL4-hUpf1 Mobility Shift Assays

U2OS cells were plated in 60 mm dishes (4×105 cells per dish), and then transfected with 0.5 μg pCMX-GAL4 or pCMX-GAL4-hUpf11019-1118, together with 4.5 μg pcDNA3.1-, HA-ATXWT, HA-ATXKI, HA-ATMWT, or HA-ATMKI plasmid DNAs. The HA-ATMKI protein contains an Asp-2870>Ala mutation that inactivates the kinase domain. Twenty hours after transfection, serum was removed from the medium, and the cells were cultured for an additional 24 h. The cells were then treated with 10% fetal bovine serum or 100 J/m2 UV-B. Where indicated, the serum-starved cells were pretreated for 30 min with 20 μM wortmannin prior to treatment with serum or UV. Cells were harvested in lysis buffer containing 25 mM Hepes, pH 7.4, 300 mM NaCl, 1.5 mM MgCl2, 1 mM EGTA, 1% Triton X-100, 20 mM β-glycerophosphate, 20 nM microcystin, 0.1 mM sodium orthovanadate, 1 mM DTT, plus protease inhibitors. For phosphatase treatment, 600 U λphosphatase was added to cellular extracts (New England Biolabs). Cell extracts were resolved on by SDS-PAGE, and were immunoblotted with α-GAL4 antibody.

RNA Isolation and Assays of NMD

Total or nuclear RNA was isolated using Trizol (Invitrogen) or the NE-PER kit (Pierce), respectively. The extent of NMD was determined by using RT-PCR to quantitate the levels of Globin and MUP mRNA as described previously (Ishigaki et al., Cell 83: 1-4 (2001)), except that 21 cycles of PCR were used when analyzing the effects of ATX-specific S and AS oligonucleotides.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention.

What is claimed is:

1. An isolated nucleic acid molecule, comprising substantially the same nucleotide sequence as SEQ ID NO:1.

2. The isolated nucleic acid molecule of claim 1, wherein said nucleic acid molecule encodes an ATX polypeptide comprising an amino acid sequence shown in SEQ ID NO:2.

3. A vector, comprising the isolated nucleic acid molecule of claim 1.

4. A host cell, comprising the vector of claim 3.

5. A method of producing an ATX polypeptide comprising:
a) growing the host cell according to claim 4 under conditions appropriate for expression of the ATX polypeptide, and
b) isolating the ATX polypeptide from the host cell or host cell growth medium.

6. An isolated oligonucleotide, comprising at least 15 contiguous nucleotides of a nucleotide sequence referenced as SEQ ID NO:11.

7. An isolated polypeptide, comprising substantially the same amino acid sequence as SEQ ID NO:2.

8. The polypeptide of claim 7, wherein said polypeptide comprises an amino acid sequence as referenced in SEQ ID NO:2.

9. An antibody, or antigen binding fragment thereof, which specifically binds to an ATX polypeptide comprising an amino acid sequence as referenced in SEQ ID NO:2.

10. A method for identifying a compound that specifically binds to an ATX polypeptide of claim 7, comprising:
a) contacting said ATX polypeptide with a compound, and
b) determining specific binding of said compound to said ATX polypeptide.

11. The method of claim 10, wherein said compound is a polypeptide.

12. A method for identifying an ATX-modulatory compound, comprising measuring the level of an ATX polypeptide in the presence of a test compound, wherein a difference in the level of said ATX polypeptide in the presence of said test compound compared to in the absence of said test compound indicating that said test compound is an ATX-modulatory compound, and wherein said ATX-modulatory compound is not caffeine or wortmannin.

13. The method of claim 12, wherein said ATX-modulatory compound decreases the level of ATX polypeptide.

14. The method of claim 12, wherein said ATX-modulatory compound increases the level of ATX polypeptide.

15. The method of claim 12, wherein said level of ATX polypeptide is measured by determining the kinase activity of said ATX polypeptide.

16. The method of claim 12, wherein said level of ATX polypeptide is measured by determining the phosphorylation of a p53 polypeptide or fragment.

17. The method of claim 12, wherein said level of ATX polypeptide is measured by determining the level of p53 polypeptide accumulation.

18. The method of claim 12, wherein said level of ATX polypeptide is measured by determining the level of non-sense mediated messenger RNA (mRNA) decay (NMD).

19. The method of claim 12, wherein said ATX-modulatory compound is an interfering RNA.

20. A method for modulating cell survival, comprising introducing a compound identified by the method of claim 12 into a cell in an amount effective to modulate survival of said cell.

21. The method of claim 20, wherein said compound decreases cell survival.

22. The method of claim 20, wherein said compound increases cell survival.

23. The method of claim 20, wherein said cell is exposed to a stressor agent.

24. The method of claim 23, wherein said stressor agent is selected from the group consisting of: UV light, ionizing radiation, and a chemical agent.

25. A method for decreasing cell survival, comprising introducing the antisense oligonucleotide according to claim 6 into a cell in an amount effective to decrease survival of said cell.


SEQUENCE LISTING (11 sequences)

SEQ ID NO: 1 - DNA, 12464 bp, Homo sapiens
  CDS (91)...(10656)
caaccttcaaattcagctgtggtgtctcggcaaaggcacgatgataccagagtccacgctgacatacagaatgacgaaaaggagagatcgatgtcttattgtgatgagtctcgactgtcgaatcttcttcggaggatcacccgggaagacgacagagaccgaagattggctactgtaaagcagttgaaagaatttattcagcaaccagaaaataagctggtactagttaaacaattggataatatcttggctgctgtacatgacgtgcttaatgaaagtagcaaattgcttcaggagttgagacaggagggagcttgctgtcttggccttctttgtgcttctctgagctatgaggctgagaagatcttcaagtggatttttagcaaatttagctcatctgcaaaagatgaagttaaactcctctacttatgtgccacctacaaagcactagagactgtaggagaaaagaaagccttttcatctgtaatgcagcttgtaatgaccagcctgcagtctattcttgaaaatgtggatacaccagaattgctttgtaaatgtgttaagtgcattcttttggtggctcgatgttaccctcatattttcagcactaattttagggatacagttgatatattagttggatggcatatagatcatactcagaaaccttcgctcacgcagcaggtatctgggtggttgcagagtttggagccattttgggtagctgatcttgcattttctactactcttcttggtcagtttctggaagacatggaagcatatgctgaggacctcagccatgtggcctctggggaatcagtggatgaagatgtccctcctccatcagtgtcattaccaaagctggctgcacttctccgggtatttagtactgtggtgaggagcattggggaacgcttcagcccaattcggggtcctccaattactgaggcatatgtaacagatgttctgtacagagtaatgagatgtgtgacggctgcaaaccaggtgtttttttctgaggctgtgttgacagctgctaatgagtgtgttggtgttttgctcggcagcttggatcctagcatgactatacattgtgacatggtcattacatatggattagaccaactggagaattgccagacttgtggtaccgattatatcatctcagtcttgaatttactcacgctgattgttgaacagataaatacgaaactgccatcatcatttgtagaaaaactgtttataccatcatctaaactactattcttgcgttatcataaagaaaaagaggttgttgctgtagcccatgctgtttatcaagcagtgctcagcttgaagaatattcctgttttggagactgcctataagttaatattgggagaaatgacttgtgccctaaacaacctcctacacagtctacaacttcctgaggcctgttctgaaataaaacatgaggcttttaagaatcatgtgttcaatgtagacaatgcaaaatttgtagttatatttgacctcagtgccctgactacaattggaaatgccaaaaactcactaatagggatgtgggcgctatctccaactgtctttgcacttctgagtaagaatctgatgattgtgcacagtgacctggctgttcacttccctgccattcagtatgctgtgctctacacattgtattctcattgtaccaggcatgatcactttatctctagtagcctcagttcttcctctccttctttgtttgatggagctgtgattagcactgtaactacggctacaaagaaacatttctcaattatattaaatcttctgggaatattacttaagaaagataaccttaaccaggacacgaggaaactgttaatgacttgggctttggaagcagctgttttaatgaagaagtctgaaacatacgcacctttattctctcttccgtctttccataaattttgcaaaggccttttagccaacactctcgttgaagatgtgaatatctgtctgcaggcatgcagcagtctacatgctctgtcctcttccttgccagatgatcttttacagagatgtgtcgatgtttgccgtgttcaactagtgcacagtggaactcgtattcgacaagcatttggaaaactgttgaaatcaattcctttagatgttgtcctaagcaataacaatcacacagaaattcaagaaatttctttagcattaagaagtcacatgagtaaagcaccaagtaatacattccacccccaagatttctctgatgttattagttttattttgtatgggaactctcatagaacagggaaggacaattggttggaaagactgttctatagctgccagagactggataagcgtgaccagtcaacaattccacgcaatctcctgaagacagatgctgtcctttggcagtgggccatatgggaagctgcacaattcactgttctttctaagctgagaaccccactgggcagagctcaagacaccttccagacaattgaaggtatcattcgaagtctcgcagctcacacattaaaccctgatcaggatgttagtcagtggacaactgcagacaatgatgaaggccatggtaacaaccaacttagacttgttcttcttctgcagtatctggaaaatctggagaaattaatgtataatgcatacgagggatgtgctaatgcattaacttcacctcccaaggtcattagaacttttttctataccaatcgccaaacttgtcaggactggctaacgcggattcgactctccatcatgagggtaggattgttggcaggccagcctgcagtgacagtgagacatggctttgacttgcttacagagatgaaaacaaccagcctatctcaggggaatgaattggaagtaaccattatgatggtggtagaagcattatgtgaacttcattgtcctgaagctatacagggaattgctgtctggtcatcatctattgttggaaaaaatcttctgtggattaactcagtggctcaacaggctgaagggaggtttgaaaaggcctctgtggagtaccaggaacacctgtgtgccatgacaggtgttgattgctgcatctccagctttgacaaatcggtgctcaccttagccaatgctgggcgtaacagtgccagcccgaaacattctctgaatggtgaatccagaaaaactgtgctgtccaaaccgactgactcttcccctgaggttataaattatttaggaaataaagcatgtgagtgctacatctcaattgccgattgggctgctgtgcaggaatggcagaacgctatccatgacttgaaaaagagtaccagtagcacttccctcaacctgaaagctgacttcaactatataaaatcattaagcagctttgagtctggaaaatttgttgaatgtaccgagcaattagaattgttaccaggagaaaatatcaatctacttgctggaggatcaaaagaaaaaatagacatgaaaaaactgcttcctaacatgttaagtccggatccgagggaacttcagaaatccattgaagttcaattgttaagaagttctgtttgtttggcaactgctttaaacccgatagaacaagatcagaagtggcagtctataactgaaaatgtggtaaagtacttgaagcaaacatcccgcatcgctattggacctctgagactttctactttaacagtttcacagtctttgccagttctaagtaccttgcagctgtattgctcatctgctttggagaacacagtttctaacagactttcaacagaggactgtcttattccactcttcagtgaagctttacgttcatgtaaacagcatgacgtgaggccatggatgcaggcattaaggtatactatgtaccagaatcagttgttggagaaaattaaagaacaaacagtcccaattagaagccatctcatggaattaggtctaacagcagcaaaatttgctagaaaacgagggaatgtgtcccttgcaacaagactgctggcacagtgcagtgaagttcagctgggaaagaccaccactgcacaggatttagtccaacattttaaaaaactatcaacccaaggtcaagtggatgaaaaatgggggcccgaacttgatattgaaaaaaccaaattgctttatacagcaggccagtcaacacatgcaatggaaatgttgagttcttgtgccatatctttctgcaagtctgtgaaagctgaatatgcagttgctaaatcaattctgacactggctaaatggatccaggcagaatggaaagagatttcaggacagctgaaacaggtttacagagctcagcaccaacagaacttcacaggtctttctactttgtctaaaaacatactcactctaatagaactgccatctgttaatacgatggaagaagagtatcctcggatcgagagtgaatctacagtgcatattggagttggagaacctgacttcattttgggacagttgtatcacctgtcttcagtacaggcacctgaagtagccaaatcttgggcagcgttggccagctgggcttataggtggggcagaaaggtggttgacaatgccagtcagggagaaggtgttcgtctgctgcctagagaaaaatctgaagttcagaatctacttccagacactataactgaggaagagaaagagagaatatatggtattcttggacaggctgtgtgtcggccggcggggattcaggatgaagatataacacttcagataactgagagtgaagacaacgaagaagatgacatggttgatgttatctggcgtcagttgatatcaagctgcccatggctttcagaacttgatgaaagtgcaactgaaggagttattaaagtgtggaggaaagttgtagatagaatattcagcctgtacaaactctcttgcagtgcatactttactttccttaaactcaacgctggtcaaattcctttagatgaggatgaccctaggctgcatttaagtcacagagtggaacagagcactgatgacatgattgtgatggccacattgcgcctgctgcggttgctcgtgaagcatgctggtgagcttcggcagtatctggagcacggcttggagacaacacccactgcaccatggagaggaattattccgcaacttttctcacgcttaaaccaccctgaagtgtatgtgcgccaaagtatttgtaaccttctctgccgtgtggctcaagattccccacatctcatattgtatcctgcaatagtgggtaccatatcgcttagtagtgaatcccaggcttcaggaaataaattttccactgcaattccaactttacttggcaatattcaaggagaagaattgctggtttctgaatgtgagggaggaagtcctcctgcatctcaggatagcaataaggatgaacctaaaagtggattaaatgaagaccaagccatgatgcaggattgttacagcaaaattgtagataagctgtcctctgcaaaccccaccatggtattacaggttcagatgctcgtggctgaactgcgcagggtcactgtgctctgggatgagctctggctgggagttttgctgcaacaacacatgtatgtcctgagacgaattcagcagcttgaagatgaggtgaagagagtccagaacaacaacaccttacgcaaagaagagaaaattgcaatcatgagggagaagcacacagctttgatgaagcccatcgtatttgctttggagcatgtgaggagtatcacagcggctcctgcagaaacacctcatgaaaaatggtttcaggataactatggtgatgccattgaaaatgccctagaaaaactgaagactccattgaaccctgcaaagcctgggagcagctggattccatttaaagagataatgctaagtttgcaacagagagcacagaaacgtgcaagttacatcttgcgtcttgaagaaatcagtccatggttggctgccatgactaacactgaaattgctcttcctggggaagtctcagccagagacactgtcacaatccatagtgtgggcggaaccatcacaatcttaccgactaaaaccaagccaaagaaacttctctttcttggatcagatgggaagagctatccttatcttttcaaaggactggaggatttacatctggatgagagaataatgcagttcctatctattgtgaataccatgtttgctacaattaatcgccaagaaacaccccggttccatgctcgacactattctgtaacaccactaggaacaagatcaggactaatccagtgggtagatggagccacacccttatttggtctttacaaacgatggcaacaacgggaagctgccttacaagcacaaaaggcccaagattcctaccaaactcctcagaatcctggaattgtaccccgtcctagtgaactttattacagtaaaattggccctgctttgaaaacagttgggcttagcctggatgtgtcccgtcgggattggcctcttcatgtaatgaaggcagtattggaagagttaatggaggccacacccccgaatctccttgccaaagagctctggtcatcttgcacaacacctgatgaatggtggagagttacgcagtcttatgcaagatctactgcagtcatgtctatggttggatacataattggccttggagacagacatctggataatgttcttatagatatgacgactggagaagttgttcacatagattacaatgtttgctttgaaaaaggtaaaagccttagagttcctgagaaagtaccttttcgaatgacacaaaacattgaaacagcactgggtgtaactggagtagaaggtgtatttaggctttcatgtgagcaggttttacacattatgcggcgtggcagagagaccctgctgacgctgctggaggcctttgtgtacgaccctctggtggactggacagcaggaggcgaggctgggtttgctggtgctgtctatggtggaggtggccagcaggccgagagcaagcagagcaagagagagatggagcgagagatcacccgcagcctgttttcttctagagtagctgagattaaggtgaactggtttaagaatagagatgagatgctggttgtgcttcccaagttggacggtagcttagatgaatacctaagcttgcaagagcaactgacagatgtggaaaaactgcagggcaaactactggaggaaatagagtttctagaaggagctgaaggggtggatcatccttctcatactctgcaacacaggtattctgagcacacccaactacagactcagcaaagagctgttcaggaagcaatccaggtgaagctgaatgaatttgaacaatggataacacattatcaggctgcattcaataatttagaagcaacacagcttgcaagcttgcttcaagagataagcacacaaatggaccttggtcctccaagttacgtgccagcaacagcctttctgcagaatgctggtcaggcccacttgattagccagtgcgagcagctggagggggaggttggtgctctcctgcagcagaggcgctccgtgctccgtggctgtctggagcaactgcatcactatgcaaccgtggccctgcagtatccgaaggccatatttcagaaacatcgaattgaacagtggaagacctggatggaagagctcatctgtaacaccacagtagagcgttgtcaagagctctataggaaatatgaaatgcaatatgctccccagccacccccaacagtgtgtcagttcatcactgccactgaaatgaccctgcagcgatacgcagcagacatcaacagcagacttattagacaagtggaacgcttgaaacaggaagctgtcactgtgccagtttgtgaagatcagttgaaagaaattgaacgttgcattaaagttttccttcatgagaatggagaagaaggatctttgagtctagcaagtgttattatttctgccctttgtacccttacaaggcgtaacctgatgatggaaggtgcagcgtcaagtgctggagaacagctggttgatctgacttctcgggatggagcctggttcttggaggaactctgcagtatgagcggaaacgtcacctgcttggttcagttactgaagcagtgccacctggtgccacaggacttagatatcccgaaccccatggaagcgtctgagacagttcacttagccaatggagtgtatacctcacttcaggaattgaattcgaatttccggcaaatcatatttccagaagcacttcgatgtttaatgaaaggggaatacacgttagaaagtatgctgcatgaactggacggtcttattgagcagaccaccgatggcgttcccctgcagactctagtggaatctcttcaggcctacttaagaaacgcagctatgggactggaagaagaaacacatgctcattacatcgatgttgccagactactacatgctcagtacggtgaattaatccaaccgagaaatggttcagttgatgaaacacccaaaatgtcagctggccagatgcttttggtagcattcgatggcatgtttgctcaagttgaaactgctttcagcttattagttgaaaagttgaacaagatggaaattcccatagcttggcgaaagattgacatcataagggaagccaggagtactcaagttaatttttttgatgatgataatcaccggcaggtgctagaagagattttctttctaaaaagactacagactattaaggagttcttcaggctctgtggtaccttttctaaaacattgtcaggatcaagttcacttgaagatcagaatactgtgaatgggcctgtacagattgtcaatgtgaaaaccctttttagaaactcttgtttcagtgaagaccaaatggccaaacctatcaaggcattcacagctgactttgtgaggcagctcttgatagggctacccaaccaagccctcggactcacactgtgcagttttatcagtgctctgggtgtagacatcattgctcaagtagaggcaaaggactttggtgccgaaagcaaagtttctgttgatgatctctgtaagaaagcggtggaacataacatccagatagggaagttctctcagctggttatgaacagggcaactgtgttagcaagttcttacgacactgcctggaagaagcatgacttggtgcgaaggctagaaaccagtatttcttcttgtaagacaagcctgcagcgggttcagctgcatattgccatgtttcagtggcaacatgaagatctacttatcaatagaccacaagccatgtcagtcacacctcccccacggtctgctatcctaaccagcatgaaaaagaagctgcataccctgagccagattgaaacttctattgcaacagttcaggagaagctagctgcacttgaatcaagtattgaacagcgactcaagtgggcaggtggtgccaaccctgcattggcccctgtactacaagattttgaagcaacgatagctgaaagaagaaatcttgtccttaaagagagccaaagagcaagtcaggtcacatttctctgcagcaatatcattcattttgaaagtttacgaacaagaactgcagaagccttaaacctggatgcggcgttatttgaactaatcaagcgatgtcagcagatgtgttcgtttgcatcacagtttaacagttcagtgtctgagttagagcttcgtttattacagagagtggacactggtcttgaacatcctattggcagctctgaatggcttttgtcagcacacaaacagttgacccaggatatgtctactcagagggcaattcagacagagaaagagcagcagatagaaacggtctgtgaaacaattcagaatctggttgataatataaagactgtgctcactggtcataaccgacagcttggagatgtcaaacatctcttgaaagctatggctaaggatgaagaagctgctctggcagatggtgaagatgttccctatgagaacagtgttaggcagtttttgggtgaatataaatcatggcaagacaacattcaaacagttctatttacattagtccaggctatgggtcaggttcgaagtcaagaacacgttgaaatgctccaggaaatcactcccaccttgaaagaactgaaaacacaaagtcagagtatctataataatttagtgagttttgcatcacccttagtcaccgatgcaacaaatgaatgttcgagtccaacgtcatctgctacttatcagccatccttcgctgcagcagtccggagtaacactggccagaagactcagcctgatgtcatgtcacagaatgctagaaagctgatccagaaaaatcttgctacatcagctgatactccaccaagcaccgttccaggaactggcaagagtgttgcttgtagtcctaaaaaggcagtcagagaccctaaaactgggaaagcggtgcaagagagaaactcctatgcagtgagtgtgtggaagagagtgaaagccaagttagagggccgagatgttgatccgaataggaggatgtcagttgctgaacaggttgactatgtcattaaggaagcaactaatctagataacttggctcagctgtatgaaggttggacagcctgggtgtgaatggcaagacagtagatgagtctggttaagcgaggtcagacatccaccagaatcaactcagcctcaggcatccaaagccacaccacagtcggtggtgatgcaactgggggcttactctgaggaaacctaggaaatctcggtgcactaggaagtgaatcccgcaggacagctgcactcagggatacgcccaacaccatggcctgcaaccccagggtcaagggtgaaggaaagcaagctcaccgcctgaacacggagattgtctttctgccacagaacagcagcagacgtgtcgggaggttagctgcggaaagaaatcgggatgccgcggagcacagagtgatttggaactccattccacctgaccctgtgtgtacaatccaggaaaaaaacaaaccccactcagaaacagagaaaactggggtcgcgaagaaatcacagccaaggaagatttgatgcattcagattctcgtgtaacacttgttgcttggcaacagtactggttgggttgaccagtaagtagaaaaaggctaaaggctatgcgatatgaatttcagaaatggactgaaaatggagagctatgtaacagatacactacagtagaagaacttacttctgaaatgaagggaaaaaaaccaccccatcgttccctactcctccccaccacttacccgttccccctttacctaatctagtagattagccatctttcaaattcacttttatttcagtccttatatttcatatacttccgtctcgatgctgttaacaacttctgataacatggaaaattcaaggattgtttaaaggtctgatgatcacacacaaaatgtaattccggttatttaagtcatttctgtgattctatcatgtacagtttccagaattgtcactgtgcattcaaaagtaatgaatctaacagacatttgatttaatgtacactcccttttgcttatagtgtgcattttttttggaggtcattcaaattttccctcttctgtgatagctgtagtttctttcatagaaagtagctaatccagtgtaatcttttacctttttaaaaaccaagatagagtatctattagagttttacattgttgatgatagattaacaataaagtgatgttctggtggaggtagactgaaatttttttaattcatgtttttcatttgatacttttaatttacacttagtaaattaaaagttgtttaatttacttggcattttaggacatgtacatgaaacagtgaaaatgagatccaccaacatcttttattaagttcagttattagtctgtgaagtgctttactttttgcacaattttaatagcttgctattcagtaatacattatagtgaattcatgatcaaggtttccttaaatttagcattgcatttcagtactgactgtgtaagctaaattgctgatccaaaataaaaacccagactagaatagggttcttaaaatcaagtatcaatacaaaatagaacacaattaaaatcttaattgttggctgggcacagtggctcacgcctgtaatcccagcactttgggaggccgaggcgggcggatcatgaggttaggagagcgagaccatcctggctaacacggtgaaaccccgtctttactaaaatacaaaaaaaattagccgggtgtggtggcgggcgcctgtagtcccagctactcgggaggctgaggcaggagaatggcgtgaacccaggaggcggagcttgcagtgagccgagattgtgccactgcactccagcctgggcaacagagctagactctgtgtcaaaaataaatgactagat

SEQ ID NO: 2 - protein, 3521 aa, Homo sapiens [sequence omitted]

SEQ ID NO: 3 - DNA, 2629 bp, Homo sapiens
  CDS (252)...(1433)
acttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggtaggggcccacttcaccgtactaaccagggaagctgtccctcatgttgaattttctctaacttcaaggcccatatctgtgaaatgctggcatttgcacctacctcacagagtgcattgtgagggttaatgaaataatgtacatctggccttgaaaccaccttttattacatggggtctaaaacttgacccccttgagggtgcctgttccctctccctctccctgttggctggtgggttggtagtttctacagttgggcagctggttaggtagagggagttgtcaagtcttgctggcccagccaaaccctgtctgacaacctcttggtcgaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtggagctggaagggtcaacatcttttacattctgcaagcacatctgcattttcaccccacccttcccctccttctccctttttatatcccatttttatatcgatctcttattttacaataaaactttgctgcca

SEQ ID NO: 4 - protein, 393 aa, Homo sapiens [sequence omitted]

SEQ ID NO: 5 - DNA, 12606 bp, Homo sapiens
  CDS (1703)...(10798)
attgacttgatgcacaacatcacaaaggcgatttttgagaactgatagtgcatcagccgacccaggtaatttaaaatattcttcatccagagatagaggtggttcttcctcttacggactgcaaccttcaaattcagctgtggtgtctcggcaaaggcacgatgataccagagtccacgctgacatacagaatgacgaaaaggagagatcgatgtcttattgtgatgagtctcgactgtcgaatcttcttcggaggatcacccgggaagacgacagagaccgaagattggctactgtaaagcagttgaaagaatttattcagcaaccagaaaataagctggtactagttaaacaattggataatatcttggctgctgtacatgacgtgcttaatgaaagtagcaaattgcttcaggagttgagacaggagggagcttgctgtcttggccttctttgtgcttctctgagctatgaggctgagaagatcttcaagtggatttttagcaaatttagctcatctgcaaaagatgaagttaaactcctctacttatgtgccacctacaaagcactagagactgtaggagaaaagaaagccttttcatctgtaatgcagcttgtaatgaccagcctgcagtctattcttgaaaatgtggatacaccagaattgctttgtaaatgtgttaagtgcattcttttggtggctcgatgttaccctcatattttcagcactaattttagggatacagttgatatattagttggatggcatatagatcatactcagaaaccttcgctcacgcagcaggtatctgggtggttgcagagtttggagccattttgggtagctgatcttgcattttctactactcttcttggtcagtttctggaagacatggaagcatatgctgaggacctcagccatgtggcctctggggaatcagtggatgaagatgtccctcctccatcagtgtcattaccaaagctggctgcacttctccgggtatttagtactgtggtgaggagcattggggaacgcttcagcccaattcggggtcctccaattactgaggcatatgtaacagatgttctgtacagagtaatgagatgtgtgacggctgcaaaccaggtgtttttttctgaggctgtgttgacagctgctaatgagtgtgttggtgttttgctcggcagcttggatcctagcatgactatacattgtgacatggtcattacatatggattagaccaactggagaattgccagacttgtggtaccgattatatcatctcagtcttgaatttactcacgctgattgttgaacagataaatacgaaactgccatcatcatttgtagaaaaactgtttataccatcatctaaactactattcttgcgttatcataaagaaaaagaggttgttgctgtagcccatgctgtttatcaagcagtgctcagcttgaagaatattcctgttttggagactgcctataagttaatattgggagaaatgacttgtgccctaaacaacctcctacacagtctacaacttcctgaggcctgttctgaaataaaacatgaggcttttaagaatcatgtgttcaatgtagacaatgcaaaatttgtagttatatttgacctcaattgactacaaaatttgtagttaaatttgactacaattggaaatgccaaaaactcactaatagggatgtgggcgctatctccaactgtctttgcacttctgagtaagaatctgatgattgtgcacagtgacctggctgttcacttccctgccattcagtatgctgtgctctacacattgtattctcattgtaccaggcatgatcactttatctctagtagcctcagttcttcctctccttctttgtttgatggagctgtgattagcactgtaactacggctacaaagaaacatttctcaattatattaaatcttctgggaatattacttaagaaagataaccttaaccaggacacgaggaaactgttaatgacttgggctttggaagcagctgttttaatgaagaagtctgaaacatacgcacctttattctctcttccgtctttccataaattttgcaaaggccttttagccaacactctcgttgaagatgtgaatatctgtctgcaggcatgcagcagtctacatgctctgtcctcttccttgccagatgatcttttacagagatgtgtcgatgtttgccgtgttcaactagtgcacagtggaactcgtattcgacaagcatttggaaaactgttgaaatcaattcctttagatgttgtcctaagcaataacaatcacacagaaattcaagaaatttctttagcattaagaagtcacatgagtaaagcaccaagtaatacattccacccccaagatttctctgatgttattagttttattttgtatgggaactctcatagaacagggaaggacaattggttggaaagactgttctatagctgccagagactggataagcgtgaccagtcaacaattccacgcaatctcctgaagacagatgctgtcctttggcagtgggccatatgggaagctgcacaattcactgttctttctaagctgagaaccccactgggcagagctcaagacaccttccagacaattgaaggtatcattcgaagtctcgcagctcacacattaaaccctgatcaggatgttagtcagtggacaactgcagacaatgatgaaggccatggtaacaaccaacttagacttgttcttcttctgcagtatctggaaaatctggagaaattaatgtataatgcatacgagggatgtgctaatgcattaacttcacctcccaaggtcattagaacttttttctataccaatcgccaaacttgtcaggactggctaacgcggattcgactctccatcatgagggtaggattgttggcaggccagcctgcagtgacagtgagacatggctttgacttgcttacagagatgaaaacaaccagcctatctcaggggaatgaattggaagtaaccattatgatggtggtagaagcattatgtgaacttcattgtcctgaagctatacagggaattgctgtctggtcatcatctattgttggaaaaaatcttctgtggattaactcagtggctcaacaggctgaagggaggtttgaaaaggcctctgtggagtaccaggaacacctgtgtgccatgacaggtgttgattgctgcatctccagctttgacaaatcggtgctcaccttagccaatgctgggcgtaacagtgccagcccgaaacattctctgaatggtgaatccagaaaaactgtgctgtccaaaccgactgactcttcccctgaggttataaattatttaggaaataaagcatgtgagtgctacatctcaattgccgattgggctgctgtgcaggaatggcagaacgctatccatgacttgaaaaagagtaccagtagcacttccctcaacctgaaagctgacttcaactatataaaatcattaagcagctttgagtctggaaaatttgttgaatgtaccgagcaattagaattgttaccaggagaaaatatcaatctacttgctggaggatcaaaagaaaaaatagacatgaaaaaactgcttcctaacatgttaagtccggatccgagggaacttcagaaatccattgaagttcaattgttaagaagttctgtttgtttggcaactgctttaaacccgatagaacaagatcagaagtggcagtctataactgaaaatgtggtaaagtacttgaagcaaacatcccgcatcgctattggacctctgagactttctactttaacagtttcacagtctttgccagttctaagtaccttgcagctgtattgctcatctgctttggagaacacagtttctaacagactttcaacagaggactgtcttattccactcttcagtgaagctttacgttcatgtaaacagcatgacgtgaggccatggatgcaggcattaaggtatactatgtaccagaatcagttgttggagaaaattaaagaacaaacagtcccaattagaagccatctcatggaattaggtctaacagcagcaaaatttgctagaaaacgagggaatgtgtcccttgcaacaagactgctggcacagtgcagtgaagttcagctgggaaagaccaccactgcacaggatttagtccaacattttaaaaaactatcaacccaaggtcaagtggatgaaaaatgggggcccgaacttgatattgaaaaaaccaaattgctttatacagcaggccagtcaacacatgcaatggaaatgttgagttcttgtgccatatctttctgcaagtctgtgaaagctgaatatgcagttgctaaatcaattctgacactggctaaatggatccaggcagaatggaaagagatttcaggacagctgaaacaggtttacagagctcagcaccaacagaacttcacaggtctttctactttgtctaaaaacatactcactctaatagaactgccatctgttaatacgatggaagaagagtatcctcggatcgagagtgaatctacagtgcatattggagttggagaacctgacttcattttgggacagttgtatcacctgtcttcagtacaggcacctgaagtagccaaatcttgggcagcgttggccagctgggcttataggtggggcagaaaggtggttgacaatgccagtcagggagaaggtgttcgtctgctgcctagagaaaaatctgaagttcagaatctacttccagacactataactgaggaagagaaagagagaatatatggtattcttggacaggctgtgtgtcggccggcggggattcaggatgaagatataacacttcagataactgagagtgaagacaacgaagaagatgacatggttgatgttatctggcgtcagttgatatcaagctgcccatggctttcagaacttgatgaaagtgcaactgaaggagttattaaagtgtggaggaaagttgtagatagaatattcagcctgtacaaactctcttgcagtgcatactttactttccttaaactcaacgctggtcaaattcctttagatgaggatgaccctaggctgcatttaagtcacagagtggaacagagcactgatgacatgattgtgatggccacattgcgcctgctgcggttgctcgtgaagcatgctggtgagcttcggcagtatctggagcacggcttggagacaacacccactgcaccatggagaggaattattccgcaacttttctcacgcttaaaccaccctgaagtgtatgtgcgccaaagtatttgtaaccttctctgccgtgtggctcaagattccccacatctcatattgtatcctgcaatagtgggtaccatatcgcttagtagtgaatcccaggcttcaggaaataaattttccactgcaattccaactttacttggcaatattcaaggagaagaattgctggtttctgaatgtgagggaggaagtcctcctgcatctcaggatagcaataaggatgaacctaaaagtggattaaatgaagaccaagccatgatgcaggattgttacagcaaaattgtagataagctgtcctctgcaaaccccaccatggtattacaggttcagatgctcgtggctgaactgcgcagggtcactgtgctctgggatgagctctggctgggagttttgctgcaacaacacatgtatgtcctgagacgaattcagcagcttgaagatgaggtgaagagagtccagaacaacaacaccttacgcaaagaagagaaaattgcaatcatgagggagaagcacacagctttgatgaagcccatcgtatttgctttggagcatgtgaggagtatcacagcggctcctgcagaaacacctcatgaaaaatggtttcaggataactatggtgatgccattgaaaatgccctagaaaaactgaagactccattgaaccctgcaaagcctgggagcagctggattccatttaaagagataatgctaagtttgcaacagagagcacagaaacgtgcaagttacatcttgcgtcttgaagaaatcagtccatggttggctgccatgactaacactgaaattgctcttcctggggaagtctcagccagagacactgtcacaatccatagtgtgggcggaaccatcacaatcttaccgactaaaaccaagccaaagaaacttctctttcttggatcagatgggaagagctatccttatcttttcaaaggactggaggatttacatctggatgagagaataatgcagttcctatctattgtgaataccatgtttgctacaattaatcgccaagaaacaccccggttccatgctcgacactattctgtaacaccactaggaacaagatcaggactaatccagtgggtagatggagccacacccttatttggtctttacaaacgatggcaacaacgggaagctgccttacaagcacaaaaggcccaagattcctaccaaactcctcagaatcctggaattgtaccccgtcctagtgaactttattacagtaaaattggccctgctttgaaaacagttgggcttagcctggatgtgtcccgtcgggattggcctcttcatgtaatgaaggcagtattggaagagttaatggaggccacacccccgaatctccttgccaaagagctctggtcatcttgcacaacacctgatgaatggtggagagttacgcagtcttatgcaagatctactgcagtcatgtctatggttggatacataattggccttggagacagacatctggataatgttcttatagatatgacgactggagaagttgttcacatagattacaatgtttgctttgaaaaaggtaaaagccttagagttcctgagaaagtaccttttcgaatgacacaaaacattgaaacagcactgggtgtaactggagtagaaggtgtatttaggctttcatgtgagcaggttttacacattatgcggcgtggcagagagaccctgctgacgctgctggaggcctttgtgtacgaccctctggtggactggacagcaggaggcgaggctgggtttgctggtgctgtctatggtggaggtggccagcaggccgagagcaagcagagcaagagagagatggagcgagagatcacccgcagcctgttttcttctagagtagctgagattaaggtgaactggtttaagaatagagatgagatgctggttgtgcttcccaagttggacggtagcttagatgaatacctaagcttgcaagagcaactgacagatgtggaaaaactgcagggcaaactactggaggaaatagagtttctagaaggagctgaaggggtggatcatccttctcatactctgcaacacaggtattctgagcacacccaactacagactcagcaaagagctgttcaggaagcaatccaggtgaagctgaatgaatttgaacaatggataacacattatcaggctgcattcaataatttagaagcaacacagcttgcaagcttgcttcaagagataagcacacaaatggaccttggtcctccaagttacgtgccagcaacagcctttctgcagaatgctggtcaggcccacttgattagccagtgcgagcagctggagggggaggttggtgctctcctgcagcagaggcgctccgtgctccgtggctgtctggagcaactgcatcactatgcaaccgtggccctgcagtatccgaaggccatatttcagaaacatcgaattgaacagtggaagacctggatggaagagctcatctgtaacaccacagtagagcgttgtcaagagctctataggaaatatgaaatgcaatatgctccccagccacccccaacagtgtgtcagttcatcactgccactgaaatgaccctgcagcgatacgcagcagacatcaacagcagacttattagacaagtggaacgcttgaaacaggaagctgtcactgtgccagtttgtgaagatcagttgaaagaaattgaacgttgcattaaagttttccttcatgagaatggagaagaaggatctttgagtctagcaagtgttattatttctgccctttgtacccttacaaggcgtaacctgatgatggaaggtgcagcgtcaagtgctggagaacagctggttgatctgacttctcgggatggagcctggttcttggaggaactctgcagtatgagcggaaacgtcacctgcttggttcagttactgaagcagtgccacctggtgccacaggacttagatatcccgaaccccatggaagcgtctgagacagttcacttagccaatggagtgtatacctcacttcaggaattgaattcgaatttccggcaaatcatatttccagaagcacttcgatgtttaatgaaaggggaatacacgttagaaagtatgctgcatgaactggacggtcttattgagcagaccaccgatggcgttcccctgcagactctagtggaatctcttcaggcctacttaagaaacgcagctatgggactggaagaagaaacacatgctcattacatcgatgttgccagactactacatgctcagtacggtgaattaatccaaccgagaaatggttcagttgatgaaacacccaaaatgtcagctggccagatgcttttggtagcattcgatggcatgtttgctcaagttgaaactgctttcagcttattagttgaaaagttgaacaagatggaaattcccatagcttggcgaaagattgacatcataagggaagccaggagtactcaagttaatttttttgatgatgataatcaccggcaggtgctagaagagattttctttctaaaaagactacagactattaaggagttcttcaggctctgtggtaccttttctaaaacattgtcaggatcaagttcacttgaagatcagaatactgtgaatgggcctgtacagattgtcaatgtgaaaaccctttttagaaactcttgtttcagtgaagaccaaatggccaaacctatcaaggcattcacagctgactttgtgaggcagctcttgatagggctacccaaccaagccctcggactcacactgtgcagttttatcagtgctctgggtgtagacatcattgctcaagtagaggcaaaggactttggtgccgaaagcaaagtttctgttgatgatctctgtaagaaagcggtggaacataacatccagatagggaagttctctcagctggttatgaacagggcaactgtgttagcaagttcttacgacactgcctggaagaagcatgacttggtgcgaaggctagaaaccagtatttcttcttgtaagacaagcctgcagcgggttcagctgcatattgccatgtttcagtggcaacatgaagatctacttatcaatagaccacaagccatgtcagtcacacctcccccacggtctgctatcctaaccagcatgaaaaagaagctgcataccctgagccagattgaaacttctattgcaacagttcaggagaagctagctgcacttgaatcaagtattgaacagcgactcaagtgggcaggtggtgccaaccctgcattggcccctgtactacaagattttgaagcaacgatagctgaaagaagaaatcttgtccttaaagagagccaaagagcaagtcaggtcacatttctctgcagcaatatcattcattttgaaagtttacgaacaagaactgcagaagccttaaacctggatgcggcgttatttgaactaatcaagcgatgtcagcagatgtgttcgtttgcatcacagtttaacagttcagtgtctgagttagagcttcgtttattacagagagtggacactggtcttgaacatcctattggcagctctgaatggcttttgtcagcacacaaacagttgacccaggatatgtctactcagagggcaattcagacagagaaagagcagcagatagaaacggtctgtgaaacaattcagaatctggttgataatataaagactgtgctcactggtcataaccgacagcttggagatgtcaaacatctcttgaaagctatggctaaggatgaagaagctgctctggcagatggtgaagatgttccctatgagaacagtgttaggcagtttttgggtgaatataaatcatggcaagacaacattcaaacagttctatttacattagtccaggctatgggtcaggttcgaagtcaagaacacgttgaaatgctccaggaaatcactcccaccttgaaagaactgaaaacacaaagtcagagtatctataataatttagtgagttttgcatcacccttagtcaccgatgcaacaaatgaatgttcgagtccaacgtcatctgctacttatcagccatccttcgctgcagcagtccggagtaacactggccagaagactcagcctgatgtcatgtcacagaatgctagaaagctgatccagaaaaatcttgctacatcagctgatactccaccaagcaccgttccaggaactggcaagagtgttgcttgtagtcctaaaaaggcagtcagagaccctaaaactgggaaagcggtgcaagagagaaactcctatgcagtgagtgtgtggaagagagtgaaagccaagttagagggccgagatgttgatccgaataggaggatgtcagttgctgaacaggttgactatgtcattaaggaagcaactaatctagataacttggctcagctgtatgaaggttggacagcctgggtgtgaatggcaagacagtagatgagtctggttaagcgaggtcagacatccaccagaatcaactcagcctcaggcatccaaagccacaccacagtcggtggtgatgcaactgggggcttactctgaggaaacctaggaaatctcggtgcactaggaagtgaatcccgcaggacagctgcactcagggatacgcccaacaccatggcctgcaaccccagggtcaagggtgaaggaaagcaagctcaccgcctgaacacggagattgtctttctgccacagaacagcagcagacgtgtcgggaggttagctgcggaaagaaatcgggatgccgcggagcacagagtgatttggaactccattccacctgaccctgtgtgtacaatccaggaaaaaaacaaaccccactcagaaacagagaaaactggggtcgcgaagaaatcacagccaaggaagatttgatgcattcagattctcgtgtaacacttgttgcttggcaacagtactggttgggttgaccagtaagtagaaaaaggctaaaggctatgcgatatgaatttcagaaatggactgaaaatggagagctatgtaacagatacactacagtagaagaacttacttctgaaatgaagggaaaaaaaccaccccatcgttccctactcctccccaccacttacccgttccccctttacctaatctagtagattagccatctttcaaattcacttttatttcagtccttatatttcatatacttccgtctcgatgctgttaacaacttctgataacatggaaaattcaaggattgtttaaaggtctgatgatcacacacaaaatgtaattccggttatttaagtcatttctgtgattctatcatgtacagtttccagaattgtcactgtgcattcaaaagtaatgaatctaacagacatttgatttaatgtacactcccttttgcttatagtgtgcattttttttggaggtcattcaaattttccctcttctgtgatagctgtagtttctttcatagaaagtagctaatccagtgtaatcttttacctttttaaaaaccaagatagagtatctattagagttttacattgttgatgatagattaacaataaagtgatgttctggtggaggtagactgaaatttttttaattcatgtttttcatttgatacttttaatttacacttagtaaattaaaagttgtttaatttacttggcattttaggacatgtacatgaaacagtgaaaatgagatccaccaacatcttttattaagttcagttattagtctgtgaagtgctttactttttgcacaattttaatagcttgctattcagtaatacattatagtgaattcatgatcaaggtttccttaaatttagcattgcatttcagtactgactgtgtaagctaaattgctgatccaaaataaaaacccagactagaatagggttcttaaaatcaagtatcaatacaaaatagaacacaattaaaatcttaattgttggctgggcacagtggctcacgcctgtaatcccagcactttgggaggccgaggcgggcggatcatgaggttaggagagcgagaccatcctggctaacacggtgaaaccccgtctttactaaaatacaaaaaaaattagccgggtgtggtggcgggcgcctgtagtcccagctactcgggaggctgaggcaggagaatggcgtgaacccaggaggcggagcttgcagtgagccgagattgtgccactgcactccagcctgggcaacagagctagactctgtgtcaaaaataaatgactagat

SEQ ID NO: 6 - protein, 3031 aa, Homo sapiens
MWALSPTVFALLSKNLMIVHSDLAVHFPAIQYAVLYTLYSHCTRHDHFISSSLSSSSPSLFDGAVISTVTTATKKHFSIILNLLGILLKKDNLNQDTRKLLMTWALEAAVLMKKSETYAPLFSLPSFHKFCKGLLANTLVEDVNICLQACSSLHALSSSLPDDLLQRCVDVCRVQLVHSGTRIRQAFGKLLKSIPLDVVLSNNNHTEIQEISLALRSHMSKAPSNTFHPQDFSDVISFILYGNSHRTGKDNWLERLFYSCQRLDKRDQSTIPRNLLKTDAVLWQWAIWEAAQFTVLSKLRTPLGRAQDTFQTIEGIIRSLAAHTLNPDQDVSQWTTADNDEGHGNNQLRLVLLLQYLENLEKLMYNAYEGCANALTSPPKVIRTFFYTNRQTCQDWLTRIRLSIMRVGLLAGQPAVTVRHGFDLLTEMKTTSLSQGNELEVTIMMVVEALCELHCPEAIQGIAVWSSSIVGKNLLWINSVAQQAEGRFEKASVEYQEHLCAMTGVDCCISSFDKSVLTLANAGRNSASPKHSLNGESRKTVLSKPTDSSPEVINYLGNKACECYISIADWAAVQEWQNAIHDLKKSTSSTSLNLKADFNYIKSLSSFESGKFVECTEQLELLPGENINLLAGGSKEKIDMKKLLPNMLSPDPRELQKSIEVQLLRSSVCLATALNPIEQDQKWQSITENVVKYLKQTSRIAIGPLRLSTLTVSQSLPVLSTLQLYCSSALENTVSNRLSTEDCLIPLFSEALRSCKQHDVRPWMQALRYTMYQNQLLEKIKEQTVPIRSHLMELGLTAAKFARKRGNVSLATRLLAQCSEVQLGKTTTAQDLVQHFKKLSTQGQVDEKWGPELDIEKTKLLYTAGQSTHAMEMLSSCAISFCKSVKAEYAVAKSILTLAKWIQAEWKEISGQLKQVYRAQHQQNFTGLSTLSKNILTLIELPSVNTMEEEYPRIESESTVHIGVGEPDFILGQLYHLSSVQAPEVAKSWAALASWAYRWGRKVVDNASQGEGVRLLPREKSEVQNLLPDTITEEEKERIYGILGQAVCRPAGIQDEDITLQITESEDNEEDDMVDVIWRQLISSCPWLSELDESATEGVIKVWRKVVDRIFSLYKLSCSAYFTFLKLNAGQIPLDEDDPRLHLSHRVEQSTDDMIVMATLRLLRLLVKHAGELRQYLEHGLETTPTAPWRGIIPQLFSRLNHPEVYVRQSICNLLCRVAQDSPHLILYPAIVGTISLSSESQASGNKFSTAIPTLLGNIQGEELLVSECEGGSPPASQDSNKDEPKSGLNEDQAMMQDCYSKIVDKLSSANPTMVLQVQMLVAELRRVTVLWDELWLGVLLQQHMYVLRRIQQLEDEVKRVQNNNTLRKEEKIAIMREKHTALMKPIVFALEHVRSITAAPAETPHEKWFQDNYGDAIENALEKLKTPLNPAKPGSSWIPFKEIMLSLQQRAQKRASYILRLEEISPWLAAMTNTEIALPGEVSARDTVTIHSVGGTITILPTKTKPKKLLFLGSDGKSYPYLFKGLEDLHLDERIMQFLSIVNTMFATINRQETPRFHARHYSVTPLGTRSGLIQWVDGATPLFGLYKRWQQREAALQAQKAQDSYQTPQNPGIVPRPSELYYSKIGPALKTVGLSLDVSRRDWPLHVMKAVLEELMEATPPNLLAKELWSSCTTPDEWWRVTQSYARSTAVMSMVGYIIGLGDRHLDNVLIDMTTGEVVHIDYNVCFEKGKSLRVPEKVPFRMTQNIETALGVTGVEGVFRLSCEQVLHIMRRGRETLLTLLEAFVYDPLVDWTAGGEAGFAGAVYGGGGQQAESKQSKREMEREITRSLFSSRVAEIKVNWFKNRDEMLVVLPKLDGSLDEYLSLQEQLTDVEKLQGKLLEEIEFLEGAEGVDHPSHTLQHRYSEHTQLQTQQRAVQEAIQVKLNEFEQWITHYQAAFNNLEATQLASLLQEISTQMDLGPPSYVPATAFLQNAGQAHLISQCEQLEGEVGALLQQRRSVLRGCLEQLHHYATVALQYPKAIFQKHRIEQWKTWMEELICNTTVERCQELYRKYEMQYAPQPPPTVCQFITATEMTLQRYAADINSRLIRQVERLKQEAVTVPVCEDQLKEIERCIKVFLHENGEEGSLSLASVIISALCTLTRRNLMMEGAASSAGEQLVDLTSRDGAWFLEELCSMSGNVTCLVQLLKQCHLVPQDLDIPNPMEASETVHLANGVYTSLQELNSNFRQIIFPEALRCLMKGEYTLESMLHELDGLIEQTTDGVPLQTLVESLQAYLRNAAMGLEEETHAHYIDVARLLHAQYGELIQPRNGSVDETPKMSAGQMLLVAFDGMFAQVETAFSLLVEKLNKMEIPIAWRKIDIIREARSTQVNFFDDDNHRQVLEEIFFLKRLQTIKEFFRLCGTFSKTLSGSSSLEDQNTVNGPVQIVNVKTLFRNSCFSEDQMAKPIKAFTADFVRQLLIGLPNQALGLTLCSFISALGVDIIAQVEAKDFGAESKVSVDDLCKKAVEHNIQIGKFSQLVMNRATVLASSYDTAWKKHDLVRRLETSISSCKTSLQRVQLHIAMFQWQHEDLLINRPQAMSVTPPPRSAILTSMKKKLHTLSQIETSIATVQEKLAALESSIEQRLKWAGGANPALAPVLQDFEATIAERRNLVLKESQRASQVTFLCSNIIHFESLRTRTAEALNLDAALFELIKRCQQMCSFASQFNSSVSELELRLLQRVDTGLEHPIGSSEWLLSAHKQLTQDMSTQRAIQTEKEQQIETVCETIQNLVDNIKTVLTGHNRQLGDVKHLLKAMAKDEEAALADGEDVPYENSVRQFLGEYKSWQDNIQTVLFTLVQAMGQVRSQEHVEMLQEITPTLKELKTQSQSIYNNLVSFASPLVTDATNECSSPTSSATYQPSFAAAVRSNTGQKTQPDVMSQNARKLIQKNLATSADTPPSTVPGTGKSVACSPKKAVRDPKTGKAVQERNSYAVSVWKRVKAKLEGRDVDPNRRMSVAEQVDYVIKEATNLDNLAQLYEGWTAWV

SEQ ID NO: 7 - DNA, 12539 bp, Homo sapiens
  CDS (141)...(10730)
gtggctacagtgtcaatggaggatctggggaaaatacttatggtcggaagtcgttggggcaagagctgagggttaacaatgtgaccagccctgagttcaccagtgttcagcatggcagtcgtgctttagccaccaaagacatgaggaaatcacaggagagatcgatgtcttattctgatgagtctcgactgtcgaatcttcttcggaggatcacccgggaagacgacagagaccgaagattggctactgtaaagcagttgaaagaatttattcagcaaccagaaaataagctggtactagttaaacaattggataatatcttggctgctgtacatgacgtgcttaatgaaagtagcaaattgcttcaggagttgagacaggagggagcttgctgtcttggccttctttgtgcttctctgagctatgaggctgagaagatcttcaagtggatttttagcaaatttagctcatctgcaaaagatgaagttaaactcctctacttatgtgccacctacaaagcactagagactgtaggagaaaagaaagccttttcatctgtaatgcagcttgtaatgaccagcctgcagtctattcttgaaaatgtggatacaccagaattgctttgtaaatgtgttaagtgcattcttttggtggctcgatgttaccctcatattttcagcactaattttagggatacagttgatatattagttggatggcatatagatcatactcagaaaccttcgctcacgcagcaggtatctgggtggttgcagagtttggagccattttgggtagctgatcttgcattttctactactcttcttggtcagtttctggaagacatggaagcatatgctgaggacctcagccatgtggcctctggggaatcagtggatgaagatgtccctcctccatcagtgtcattaccaaagctggctgcacttctccgggtatttagtactgtggtgaggagcattggggaacgcttcagcccaattcggggtcctccaattactgaggcatatgtaacagatgttctgtacagagtaatgagatgtgtgacggctgcaaaccaggtgtttttttctgaggctgtgttgacagctgctaatgagtgtgttggtgttttgctcggcagcttggatcctagcatgactatacattgtgacatggtcattacatatggattagaccaactggagaattgccagacttgtggtaccgattatatcatctcagtcttgaatttactcacgctgattgttgaacagataaatacgaaactgccatcatcatttgtagaaaaactgtttataccatcatctaaactactattcttgcgttatcataaagaaaaagaggttgttgctgtagcccatgctgtttatcaagcagtgctcagcttgaagaatattcctgttttggagactgcctataagttaatattgggagaaatgacttgtgccctaaacaacctcctacacagtctacaacttcctgaggcctgttctgaaataaaacatgaggcttttaagaatcatgtgttcaatgtagacaatgcaaaatttgtagttaaatttgacctcagtgccctgactacaattggaaatgccaaaaactcactaatagggatgtgggcgctatctccaactgtctttgcacttctgagtaagaatctgatgattgtgcacagtgacctggctgttcacttccctgccattcagtatgctgtgctctacacattgtattctcattgtaccaggcatgatcactttatctctagtagcctcagttctgcctctccttctttgtttgatggagctgtgattagcactgtaactacggctacaaagaaacatttctcaattatattaaatcttctgggaatattacttaagaaagataaccttaaccaggacacgaggaaactgttaatgacttgggctttggaagcagctgttttaatgaggaagtctgaaacatacgcacctttattctctcttccgtctttccataaattttgcaaaggccttttagccaacactctcgttgaagatgtgaatatctgtctgcaggcatgcagcagtctacatgctctgtcctcttccttgccagatgatcttttacagagatgtgtcgatgtttgccgtgttcaactagtgcacagtggaactcgtattcgacaagcatttggaaaactgttgaaatcaattcctttagatgttgtcctaagcaataacaatcacacagaaattcaagaaatttctttagcattaagaagtcacatgagtaaagcaccaagtaatacattccacccccaagatttctctgatgttattagttttattttgtatgggaactctcatagaacagggaaggacaattggttggaaagactgttctatagctgccagagactggataagcgtgaccagtcaacaattccacgcaatctcctgaagacagatgctgtcctttggcagtgggccatatgggaagctgcacaattcactgttctttctaagctgagaaccccactgggcagagctcaagacaccttccagacaattgaaggtatcattcgaagtctcgcagctcacacattaaaccctgatcaggatgttagtcagtggacaactgcagacaatgatgaaggccatggtaacaaccaacttagacttgttcttcttctgcagtatctggaaaatctggagaaattaatgtataatgcatacgagggatgtgctaatgcattaacttcacctcccaaggtcattagaacttttttctataccaatcgccaaacttgtcaggactggctaacgcggattcgactctccatcatgagggtaggattgttggcaggccagcctgcagtgacagtgagacatggctttgacttgcttacagagatgaaaacaaccagcctatctcaggggaatgaattggaagtaaccattatgatggtggtagaagcattatgtgaacttcattgtcctgaagctatacagggaattgctgtctggtcatcatctattgttggaaaaaatcttctgtggattaactcagtggctcaacaggctgaagggaggtttgaaaaggcctctgtggagtaccaggaacacctgtgtgccatgacaggtgttgattgctgcatctccagctttgacaaatcggtgctcaccttagccaatgctgggcgtaacagtgccagcccgaaacattctctgaatggtgaatccagaaaaactgtgctgtccaaaccgactgactcttcccctgaggttataaattatttaggaaataaagcatgtgagttctacatctcaattgccgattgggctgctgtgcaggaatggcagaacgctatccatgacttgaaaaagagtaccagtagcacttccctcaacctgaaagctgacttcaactatataaaatcattaagcagctttgagtctggaaaatttgttgaatgtaccgagcagttagaattgttaccaggagaaaatatcaatctacttgctggaggatcaaaagaaaaaatagacatgaaaaaactgcttcctaacatgttaagtccggatccgagggaacttcagaaatccattgaagttcaattgttaagaagttctgtttgtttggcaactgctttaaacccgatagaacaagatcagaagtggcagtctataactgaaaatgtggtaaagtacttgaagcaaacatcccgcatcgctattggacctctgagactttctactttaacagtttcacagtctttgccagttctaagtaccttgcagctgtattgctcatctgctttggagaacacagtttctaacagactttcaacagaggactgtcttattccactcttcagtgaagctttacgttcatgtaaacagcatgacgtgaggccatggatgcaggcattaaggtatactatgtaccagaatcagttgttggagaaaattaaagaacaaacagtcccaattagaagccatctcatggaattaggtctaacagcagcaaaatttgctagaaaacgagggaatgtgtcccttgcaacaagactgctggcacagtgcagtgaagttcagctgggaaagaccaccactgcacaggatttagtccaacattttaaaaaactatcaacccaaggtcaagtggatgaaaaatgggggcccgaacttgatattgaaaaaaccaaattgctttatacagcaggccagtcaacacatgcaatggaaatgttgagttcttgtgccatatctttctgcaagtctgtgaaagctgaatatgcagttgctaaatcaattctgacactggctaaatggatccaggcagaatggaaagagatttcaggacagctgaaacaggtttacagagctcagcaccaacagaacttcacaggtctttctactttgtctaaaaacatactcactctaatagaactgccatctgttaatacgatggaagaagagtatcctcggatcgagagtgaatctacagtgcatattggagttggagaacctgacttcattttgggacagttgtatcacctgtcttcagtacaggcacctgaagtagccaaatcttgggcagcgttggccagctgggcttataggtggggcagaaaggtggttgacaatgccagtcagggagaaggtgttcgtctgctgcctagagaaaaatctgaagttcagaatctacttccagacactataactgaggaagagaaagagagaatatatggtattcttggacaggctgtgtgtcggccggcggggattcaggatgaagatataacacttcagataactgagagtgaagacaacgaagaagatgacatggttgatgttatctggcgtcagttgatatcaagctgcccatggctttcagaacttgatgaaagtgcaactgaaggagttattaaagtgtggaggaaagttgtagatagaatattcagcctgtacaaactctcttgcagtgcatactttactttccttaaactcaacgctggtcaaattcctttagatgaggatgaccctaggctgcatttaagtcacagagtggaacagagcactgatgacatgattgtgatggccacattgcgcctgctgcggttgctcgtgaagcatgctggtgagcttcggcagtatctggagcacggcttggagacaacacccactgcaccatggaggggaattattccgcaacttttctcacgcttaaaccaccctgaagtgtatgtgcgccaaagtatttgtaaccttctctgccgtgtggctcaagattccccacatctcatattgtatcctgcaatagtgggtaccatatcgcttagtagtgaatcccaggcttcaggaaataaattttccactgcaattccaactttacttggcaatattcaaggagaagaattgctggtttctgaatgtgagggaggaagtcctcctgcatctcaggatagcaataaggatgaacctaaaagtggattaaatgaagaccaagccatgatgcaggattgttacagcaaaattgtagataagctgtcctctgcaaaccccaccatggtattacaggttcagatgctcgtggctgaactgcgcagggtcactgtgctctgggatgagctctggctgggagttttgctgcaacaacacatgtatgtcctgagacgaattcagcagcttgaagatgaggtgaagagagtccagaacaacaacaccttacgcaaagaagagaaaattgcaatcatgagggagaggcacacagctttgatgaagcccatcgtatttgctttggagcatgtgaggagtatcacagcggctcctgcagaaacacctcatgaaaaatggtttcaggataactatggtgatgccattgaaaatgccctagaaaaactgaagactccattgaaccctgcaaagcctgggagcagctggattccatttaaagagataatgctaagtttgcaacagagagcacagaaacgtgcaagttacatcttgcgtcttgaagaaatcagtccatggttggctgccatgactaacactgaaattgctcttcctggggaagtctcagccagagacactgtcacaatccatagtgtgggcggaaccatcacaatcttaccgactaaaaccaagccaaagaaacttctctttcttggatcagatgggaagagctatccttatcttttcaaaggactggaggatttacatctggatgagagaataatgcagttcctatctattgtgaataccatgtttgctacaattaatcgccaagaaacaccccggttccatgctcgacactattctgtaacaccactaggaacaagatcaggactaatccagtgggtagatggagccacacccttatttggtctttacaaacgatggcaacaacgggaagctgccttacaagcacaaaaggcccaagattcctaccaaactcctcagaatcctggaattgtaccccgtcctagtgaactttattacagtaaaattggccctgctttgaaaacagttgggcttagcctggatgtgtcccgtcgggattggcctcttcatgtaatgaaggcagtattggaagagttaatggaggccacacccccgaatctccttgccaaagagctctggtcatcttgcacaacacctgatgaatggtggagagttacgcagtcttatgcaagatctactgcagtcatgtctatggttggatacataattggccttggagacagacatctggataatgttcttatagatatgacgactggagaagttgttcacatagattacaatgtttgctttgaaaaaggtaaaagccttagagttcctgagaaagtaccttttcgaatgacacaaaacattgaaacagcactgggtgtaactggagtagaaggtgtatttaggctttcatgtgagcaggttttacacattatgcggcgtggcagagagaccctgctgacgctgctggaggcctttgtgtacgaccctctggtggactggacagcaggaggcgaggctgggtttgctggtgctgtctatggtggaggtggccagcaggccgagagcaagcagagcaagagagagatggagcgagagatcacccgcagcctgttttcttctagagtagctgagattaaggtgaactggtttaagaatagagatgagatgctggttgtgcttcccaagttggacggtagcttagatgaatacctaagcttgcaagagcaactgacagatgtggaaaaactgcagggcaaactactggaggaaatagagtttctagaaggagctgaaggggtggatcatccttctcatactctgcaacacaggtattctgagcacacccaactacagactcagcaaagagctgttcaggaagcaatccaggtgaagctgaatgaatttgaacaatggataacacattatcaggctgcattcaataatttagaagcaacacagcttgcaagcttgcttcaagagataagcacacaaatggaccttggtcctccaagttacgtgccagcaacagcctttctgcagaatgctggtcaggcccacttgattagccagtgcgagcagctggagggggaggttggtgctctcctgcagcagaggcgctccgtgctccgtggctgtctggagcaactgcatcactatgcaaccgtggccctgcagtatccgaaggccatatttcagaaacatcgaattgaacagtggaagacctggatggaagagctcatctgtaacaccacagtagagcgttgtcaagagctctataggaaatatgaaatgcaatatgctccccagccacccccaacagtgtgtcagttcatcactgccactgaaatgaccctgcagcgatacgcagcagacatcaacagcagacttattagacaagtggaacgcttgaaacaggaagctgtcactgtgccagtttgtgaagatcagttgaaagaaattgaacgttgcattaaagttttccttcatgagaatggagaagaaggatctttgagtctagcaagtgttattatttctgccctttgtacccttacaaggcgtaacctgatgatggaaggtgcagcgtcaagtgctggagaacagctggttgatctgacttctcgggatggagcctggttcttggaggaactctgcagtatgagcggaaacgtcacctgcttggttcagttactgaagcagtgccacctggtgccacaggacttagatatcccgaaccccatggaagcgtctgagacagttcacttagccaatggagtgtatacctcacttcaggaattgaattcgaatttccggcaaatcatatttccagaagcacttcgatgtttaatgaaaggggaatacacgttagaaagtatgctgcatgaactggacggtcttattgagcagaccaccgatggcgttcccctgcagactctagtggaatctcttcaggcctacttaagaaacgcagctatgggactggaagaagaaacacatgctcattacatcgatgttgccagactactacatgctcagtacggtgaattaatccaaccgagaaatggttcagttgatgaaacacccaaaatgtcagctggccagatgcttttggtagcattcgatggcatgtttgctcaagttgaaactgctttcagcttattagttgaaaagttgaacaagatggaaattcccatagcttggcgaaagattgacatcataagggaagccaggagtactcaagttaatttttttgatgatgataatcaccggcaggtgctagaagagattttctttctaaaaagactacagactattaaggagttcttcaggctctgtggtaccttttctaaaacattgtcaggatcaagttcacttgaagatcagaatactgtgaatgggcctgtacagattgtcaatgtgaaaaccctttttagaaactcttgtttcagtgaagaccaaatggccaaacctatcaaggcattcacagctgactttgtgaggcagctcttgatagggctacccaaccaagccctcggactcacactgtgcagttttatcagtgctctgggtgtagacatcattgctcaagtagaggcaaaggactttggtgccgaaagcaaagtttctgttgatgatctctgtaagaaagcggtggaacataacatccagatagggaagttctctcagctggttatgaacagggcaactgtgttagcaagttcttacgacactgcctggaagaagcatgacttggtgcgaaggctagaaaccagtatttcttcttgtaagacaagcctgcagcgggttcagctgcatattgccatgtttcagtggcaacatgaagatctacttatcaatagaccacaagccatgtcagtcacacctcccccacggtctgctatcctaaccagcatgaaaaagaagctgcataccctgagccagattgaaacttctattgcgacagttcaggagaagctagctgcacttgaatcaagtattgaacagcgactcaagtgggcaggtggtgccaaccctgcattggcccctgtactacaagattttgaagcaacgatagctgaaagaagaaatcttgtccttaaagagagccaaagagcaagtcaggtcacatttctctgcagcaatatcattcattttgaaagtttacgaacaagaactgcagaagccttaaacctggatgcggcgttatttgaactaatcaagcgatgtcagcagatgtgttcgtttgcatcacagtttaacagttcagtgtctgagttagagcttcgtttattacagagagtggacactggtcttgaacatcctattggcagctctgaatggcttttgtcagcacacaaacagttgacccaggatatgtctactcagagggcaattcagacagagaaagagcagcagatagaaacggtctgtgaaacaattcagaatctggttgataatataaagactgtgctcactggtcataaccgacagcttggagatgtcaaacatctcttgaaagctatggctaaggatgaagaagctgctctggcagatggtgaagatgttccctatgagaacagtgttaggcagtttttgggtgaatataaatcatggcaagacaacattcaaacagttctatttacattagtccaggctatgggtcaggttcgaagtcaagaacacgttgaaatgctccaggaaatcactcccaccttgaaagaactgaaaacacaaagtcagagtatctataataatttagtgagttttgcatcacccttagtcaccgatgcaacaaatgaatgttcgagtccaacgtcatctgctacttatcagccatccttcgctgcagcagtccggagtaacactggccagaagactcagcctgatgtcatgtcacagaatgctagaaagctgatccagaaaaatcttgctacatcagctgatactccaccaagcaccgttccaggaactggcaagagtgttgcttgtagtcctaaaaaggcagtcagagaccctaaaactgggaaagcggtgcaagagagaaactcctatgcagtgagtgtgtggaagagagtgaaagccaagttagagggccgagatgttgatccgaataggaggatgtcagttgctgaacaggttgactatgtcattaaggaagcaactaatctagataacttggctcagctgtatgaaggttggacagcctgggtgtgaatggcaagacagtagatgagtctggttaagcgaggtcagacatccaccagaatcaactcagcctcaggcatccaaagccacaccacagtcggtggtgatgcaactgggggcttactctgaggaaacctaggaaatctcggtgcactaggaagtgaatcccgcaggacagctgcactcagggatacgcccaacaccatggcctgcaaccccagggtcaagggtgaaggaaagcaaagctcaccgcctgaacacggagattgtctttctgccacagaacagcagcagacgtgtcgggaggttagctgcggaaagaaatcgggatgccgcggagcacagagtgatttggaactccattccacctgaccctgtgtgtacaatccaggaaaaaaacaaaccccactcagaaacagagaaaactggggtcgcgaagaaatcacagccaaggaagatttgatgcattcagattctcgtgtaacacttgttgcttggcaacagtactggttgggttgaccagtaagtagaaaaaggctaaaggctatgcgatatgaatttcagaaatggactgaaaatggagagctatgtaacagatacactacagtagaagaacttacttctgaaatgaagggaaaaaaaccaccccatcgttccctactcctccccaccacttacccgttccccctttacctaatctagtagattagccatctttcaaattcacttttatttcagtccttatatttcatatacttccgtctcgatgctgttaacaacttctgataacatggaaaattcaaggattgtttaaaggtctgatgatcacacacaaaatgtaattccggttatttaagtcatttctgtgattctatcatgtacagtttccagaattgtcactgtgcattcaaaagtaatgaatctaacagacatttgatttaatgtacactcccttttgcttatagtgtgcattttttttggaggtcattcaaattttccctcttctgtgatagctgtagtttctttcatagaaagtagctaatccagtgtaatcttttacctttttaaaaaccaagatagagtatctattagagttttacattgttgatgatagattaacaataaagtgatgttctggtggaggtagactgaaatttttttaattcatgtttttcatttgatacttttaatttacacttagtaaattaaaagttgtttaatttacttggcattttaggacatgtacatgaaacagtgaaaatgagatccaccaacatcttttattaagttcagttattagtctgtgaagtgctttactttttgcacaattttaatagcttgctattcagtaatacattatagtgaattcatgatcaaggtttccttaaatttagcattgcatttcagtactgactgtgtaagctaaattgctgatccaaaataaaaacccagactagaatagggttcttaaaatcaagtatcaatacaaaatagaacacaattaaaatcttaattgttggctgggcacagtggctcacgcctgtaatcccagcactttgggaggccgaggcgggcggatcatgaggttaggagagcgagaccatcctggctaacacggtgaaaccccgtctttactaaaatacaaaaaaaattagccgggtgtggtggcgggcgcctgtagtcccagctactcgggaggctgaggcaggagaatggcgtgaacccaggaggcggagcttgcagtgagccgagattgtgccactgcactccagcctgggcaacagagctagactctgtgtcaaaaataaatgactagat

SEQ ID NO: 8 - protein, 3529 aa, Homo sapiens
MRKSQERSMSYSDESRLSNLLRRITREDDRDRRLATVKQLKEFIQQPENKLVLVKQLDNILAAVHDVLNESSKLLQELRQEGACCLGLLCASLSYEAEKIFKWIFSKFSSSAKDEVKLLYLCATYKALETVGEKKAFSSVMQLVMTSLQSILENVDTPELLCKCVKCILLVARCYPHIFSTNFRDTVDILVGWHIDHTQKPSLTQQVSGWLQSLEPFWVADLAFSTTLLGQFLEDMEAYAEDLSHVASGESVDEDVPPPSVSLPKLAALLRVFSTVVRSIGERFSPIRGPPITEAYVTDVLYRVMRCVTAANQVFFSEAVLTAANECVGVLLGSLDPSMTIHCDMVITYGLDQLENCQTCGTDYIISVLNLLTLIVEQINTKLPSSFVEKLFIPSSKLLFLRYHKEKEVVAVAHAVYQAVLSLKNIPVLETAYKLILGEMTCALNNLLHSLQLPEACSEIKHEAFKNHVFNVDNAKFVVKFDLSALTTIGNAKNSLIGMWALSPTVFALLSKNLMIVHSDLAVHFPAIQYAVLYTLYSHCTRHDHFISSSLSSASPSLFDGAVISTVTTATKKHFSIILNLLGILLKKDNLNQDTRKLLMTWALEAAVLMRKSETYAPLFSLPSFHKFCKGLLANTLVEDVNICLQACSSLHALSSSLPDDLLQRCVDVCRVQLVHSGTRIRQAFGKLLKSIPLDVVLSNNNHTEIQEISLALRSHMSKAPSNTFHPQDFSDVISFILYGNSHRTGKDNWLERLFYSCQRLDKRDQSTIPRNLLKTDAVLWQWAIWEAAQFTVLSKLRTPLGRAQDTFQTIEGIIRSLAAHTLNPDQDVSQWTTADNDEGHGNNQLRLVLLLQYLENLEKLMYNAYEGCANALTSPPKVIRTFFYTNRQTCQDWLTRIRLSIMRVGLLAGQPAVTVRHGFDLLTEMKTTSLSQGNELEVTIMMVVEALCELHCPEAIQGIAVWSSSIVGKNLLWINSVAQQAEGRFEKASVEYQEHLCAMTGVDCCISSFDKSVLTLANAGRNSASPKHSLNGESRKTVLSKPTDSSPEVINYLGNKACEFYISIADWAAVQEWQNAIHDLKKSTSSTSLNLKADFNYIKSLSSFESGKFVECTEQLELLPGENINLLAGGSKEKIDMKKLLPNMLSPDPRELQKSIEVQLLRSSVCLATALNPIEQDQKWQSITENVVKYLKQTSRIAIGPLRLSTLTVSQSLPVLSTLQLYCSSALENTVSNRLSTEDCLIPLFSEALRSCKQHDVRPWMQALRYTMYQNQLLEKIKEQTVPIRSHLMELGLTAAKFARKRGNVSLATRLLAQCSEVQLGKTTTAQDLVQHFKKLSTQGQVDEKWGPELDIEKTKLLYTAGQSTHAMEMLSSCAISFCKSVKAEYAVAKSILTLAKWIQAEWKEISGQLKQVYRAQHQQNFTGLSTLSKNILTLIELPSVNTMEEEYPRIESESTVHIGVGEPDFILGQLYHLSSVQAPEVAKSWAALASWAYRWGRKVVDNASQGEGVRLLPREKSEVQNLLPDTITEEEKERIYGILGQAVCRPAGIQDEDITLQITESEDNEEDDMVDVIWRQLISSCPWLSELDESATEGVIKVWRKVVDRIFSLYKLSCSAYFTFLKLNAGQIPLDEDDPRLHLSHRVEQSTDDMIVMATLRLLRLLVKHAGELRQYLEHGLETTPTAPWRGIIPQLFSRLNHPEVYVRQSICNLLCRVAQDSPHLILYPAIVGTISLSSESQASGNKFSTAIPTLLGNIQGEELLVSECEGGSPPASQDSNKDEPKSGLNEDQAMMQDCYSKIVDKLSSANPTMVLQVQMLVAELRRVTVLWDELWLGVLLQQHMYVLRRIQQLEDEVKRVQNNNTLRKEEKIAIMRERHTALMKPIVFALEHVRSITAAPAETPHEKWFQDNYGDAIENALEKLKTPLNPAKPGSSWIPFKEIMLSLQQRAQKRASYILRLEEISPWLAAMTNTEIALPGEVSARDTVTIHSVGGTITILPTKTKPKKLLFLGSDGKSYPYLFKGLEDLHLDERIMQFLSIVNTMFATINRQETPRFHARHYSVTPLGTRSGLIQWVDGATPLFGLYKRWQQREAALQAQKAQDSYQTPQNPGIVPRPSELYYSKIGPALKTVGLSLDVSRRDWPLHVMKAVLEELMEATPPNLLAKELWSSCTTPDEWWRVTQSYARSTAVMSMVGYIIGLGDRHLDNVLIDMTTGEVVHIDYNVCFEKGKSLRVPEKVPFRMTQNIETALGVTGVEGVFRLSCEQVLHIMRRGRETLLTLLEAFVYDPLVDWTAGGEAGFAGAVYGGGGQQAESKQSKREMEREITRSLFSSRVAEIKVNWFKNRDEMLVVLPKLDGSLDEYLSLQEQLTDVEKLQGKLLEEIEFLEGAEGVDHPSHTLQHRYSEHTQLQTQQRAVQEAIQVKLNEFEQWITHYQAAFNNLEATQLASLLQEISTQMDLGPPSYVPATAFLQNAGQAHLISQCEQLEGEVGALLQQRRSVLRGCLEQLHHYATVALQYPKAIFQKHRIEQWKTWMEELICNTTVERCQELYRKYEMQYAPQPPPTVCQFITATEMTLQRYAADINSRLIRQVERLKQEAVTVPVCEDQLKEIERCIKVFLHENGEEGSLSLASVIISALCTLTRRNLMMEGAASSAGEQLVDLTSRDGAWFLEELCSMSGNVTCLVQLLKQCHLVPQDLDIPNPMEASETVHLANGVYTSLQELNSNFRQIIFPEALRCLMKGEYTLESMLHELDGLIEQTTDGVPLQTLVESLQAYLRNAAMGLEEETHAHYIDVARLLHAQYGELIQPRNGSVDETPKMSAGQMLLVAFDGMFAQVETAFSLLVEKLNKMEIPIAWRKIDIIREARSTQVNFFDDDNHRQVLEEIFFLKRLQTIKEFFRLCGTFSKTLSGSSSLEDQNTVNGPVQIVNVKTLFRNSCFSEDQMAKPIKAFTADFVRQLLIGLPNQALGLTLCSFISALGVDIIAQVEAKDFGAESKVSVDDLCKKAVEHNIQIGKFSQLVMNRATVLASSYDTAWKKHDLVRRLETSISSCKTSLQRVQLHIAMFQWQHEDLLINRPQAMSVTPPPRSAILTSMKKKLHTLSQIETSIATVQEKLAALESSIEQRLKWAGGANPALAPVLQDFEATIAERRNLVLKESQRASQVTFLCSNIIHFESLRTRTAEALNLDAALFELIKRCQQMCSFASQFNSSVSELELRLLQRVDTGLEHPIGSSEWLLSAHKQLTQDMSTQRAIQTEKEQQIETVCETIQNLVDNIKTVLTGHNRQLGDVKHLLKAMAKDEEAALADGEDVPYENSVRQFLGEYKSWQDNIQTVLFTLVQAMGQVRSQEHVEMLQEITPTLKELKTQSQSIYNNLVSFASPLVTDATNECSSPTSSATYQPSFAAAVRSNTGQKTQPDVMSQNARKLIQKNLATSADTPPSTVPGTGKSVACSPKKAVRDPKTGKAVQERNSYAVSVWKRVKAKLEGRDVDPNRRMSVAEQVDYVIKEATNLDNLAQLYEGWTAWV

SEQ ID NO: 9 - DNA, 13110 bp, Homo sapiens
  CDS (328)...(11301)
ggggaagcagtggccgtgtgagcgtgaggagctgccgccaccgcctgctcctcgtcctcctcgtcctccggggccccagcgtcgtgggccgcgcacggccctggaagagacgtcgcctcgccttcatccgcctctctcaccgcgccgctccctcgtcctgccctgcgggctcaggcggaacccggaacggccgtcctcttcccccgccctccgccgccgcctcctcctcctccttctcggcttcctcctcagccccgggccggagcggggtgtcggcggcggccggttcgggcggcggcgcttggccatgtcgtgtcggggaaggtaatgagccgcagagccccggggtctcggctgagcagcggcggcaccaactattcgcggagctggaatgactggcaacccagaactgatagtgcatcagctgacccaggtaatttaaaatattcttcatccagagatagaggtggttcttcctcttacggactgcaaccttcaaattcagctgtggtgtctcggcaaaggcacgatgataccagagtccacgctgacatacagaatgacgaaaagggtggctacagtgtcaatggaggatctggggaaaatacttatggtcggaagtcgttggggcaagagctgagggttaacaatgtgaccagccctgagttcaccagtgttcagcatggcagtcgtgctttagccaccaaagacatgaggaaatcacaggagagatcgatgtcttattctgatgagtctcgactgtcgaatcttcttcggaggatcacccgggaagacgacagagaccgaagattggctactgtaaagcagttgaaagaatttattcagcaaccagaaaataagctggtactagttaaacaattggataatatcttggctgctgtacatgacgtgcttaatgaaagtagcaaattgcttcaggagttgagacaggagggagcttgctgtcttggccttctttgtgcttctctgagctatgaggctgagaagatcttcaagtggatttttagcaaatttagctcatctgcaaaagatgaagttaaactcctctacttatgtgccacctacaaagcactagagactgtaggagaaaagaaagccttttcatctgtaatgcagcttgtaatgaccagcctgcagtctattcttgaaaatgtggatacaccagaattgctttgtaaatgtgttaagtgcattcttttggtggctcgatgttaccctcatattttcagcactaattttagggatacagttgatatattagttggatggcatatagatcatactcagaaaccttcgctcacgcagcaggtatctgggtggttgcagagtttggagccattttgggtagctgatcttgcattttctactactcttcttggtcagtttctggaagacatggaagcatatgctgaggacctcagccatgtggcctctggggaatcagtggatgaagatgtccctcctccatcagtgtcattaccaaagctggctgcacttctccgggtatttagtactgtggtgaggagcattggggaacgcttcagcccaattcggggtcctccaattactgaggcatatgtaacagatgttctgtacagagtaatgagatgtgtgacggctgcaaaccaggtgtttttttctgaggctgtgttgacagctgctaatgagtgtgttggtgttttgctcggcagcttggatcctagcatgactatacattgtgacatggtcattacatatggattagaccaactggagaattgccagacttgtggtaccgattatatcatctcagtcttgaatttactcacgctgattgttgaacagataaatacgaaactgccatcatcatttgtagaaaaactgtttataccatcatctaaactactattcttgcgttatcataaagaaaaagaggttgttgctgtagcccatgctgtttatcaagcagtgctcagcttgaagaatattcctgttttggagactgcctataagttaatattgggagaaatgacttgtgccctaaacaacctcctacacagtctacaacttcctgaggcctgttctgaaataaaacatgaggcttttaagaatcatgtgttcaatgtagacaatgcaaaatttgtagttaaatttgacctcagtgccctgactacaattggaaatgccaaaaactcactaatagggatgtgggcgctatctccaactgtctttgcacttctgagtaagaatctgatgattgtgcacagtgacctggctgttcacttccctgccattcagtatgctgtgctctacacattgtattctcattgtaccaggcatgatcactttatctctagtagcctcagttctgcctctccttctttgtttgatggagctgtgattagcactgtaactacggctacaaagaaacatttctcaattatattaaatcttctgggaatattacttaagaaagataaccttaaccaggacacgaggaaactgttaatgacttgggctttggaagcagctgttttaatgaggaagtctgaaacatacgcacctttattctctcttccgtctttccataaattttgcaaaggccttttagccaacactctcgttgaagatgtgaatatctgtctgcaggcatgcagcagtctacatgctctgtcctcttccttgccagatgatcttttacagagatgtgtcgatgtttgccgtgttcaactagtgcacagtggaactcgtattcgacaagcatttggaaaactgttgaaatcaattcctttagatgttgtcctaagcaataacaatcacacagaaattcaagaaatttctttagcattaagaagtcacatgagtaaagcaccaagtaatacattccacccccaagatttctctgatgttattagttttattttgtatgggaactctcatagaacagggaaggacaattggttggaaagactgttctatagctgccagagactggataagcgtgaccagtcaacaattccacgcaatctcctgaagacagatgctgtcctttggcagtgggccatatgggaagctgcacaattcactgttctttctaagctgagaaccccactgggcagagctcaagacaccttccagacaattgaaggtatcattcgaagtctcgcagctcacacattaaaccctgatcaggatgttagtcagtggacaactgcagacaatgatgaaggccatggtaacaaccaacttagacttgttcttcttctgcagtatctggaaaatctggagaaattaatgtataatgcatacgagggatgtgctaatgcattaacttcacctcccaaggtcattagaacttttttctataccaatcgccaaacttgtcaggactggctaacgcggattcgactctccatcatgagggtaggattgttggcaggccagcctgcagtgacagtgagacatggctttgacttgcttacagagatgaaaacaaccagcctatctcaggggaatgaattggaagtaaccattatgatggtggtagaagcattatgtgaacttcattgtcctgaagctatacagggaattgctgtctggtcatcatctattgttggaaaaaatcttctgtggattaactcagtggctcaacaggctgaagggaggtttgaaaaggcctctgtggagtaccaggaacacctgtgtgccatgacaggtgttgattgctgcatctccagctttgacaaatcggtgctcaccttagccaatgctgggcgtaacagtgccagcccgaaacattctctgaatggtgaatccagaaaaactgtgctgtccaaaccgactgactcttcccctgaggttataaattatttaggaaataaagcatgtgagttctacatctcaattgccgattgggctgctgtgcaggaatggcagaacgctatccatgacttgaaaaagagtaccagtagcacttccctcaacctgaaagctgacttcaactatataaaatcattaagcagctttgagtctggaaaatttgttgaatgtaccgagcagttagaattgttaccaggagaaaatatcaatctacttgctggaggatcaaaagaaaaaatagacatgaaaaaactgcttcctaacatgttaagtccggatccgagggaacttcagaaatccattgaagttcaattgttaagaagttctgtttgtttggcaactgctttaaacccgatagaacaagatcagaagtggcagtctataactgaaaatgtggtaaagtacttgaagcaaacatcccgcatcgctattggacctctgagactttctactttaacagtttcacagtctttgccagttctaagtaccttgcagctgtattgctcatctgctttggagaacacagtttctaacagactttcaacagaggactgtcttattccactcttcagtgaagctttacgttcatgtaaacagcatgacgtgaggccatggatgcaggcattaaggtatactatgtaccagaatcagttgttggagaaaattaaagaacaaacagtcccaattagaagccatctcatggaattaggtctaacagcagcaaaatttgctagaaaacgagggaatgtgtcccttgcaacaagactgctggcacagtgcagtgaagttcagctgggaaagaccaccactgcacaggatttagtccaacattttaaaaaactatcaacccaaggtcaagtggatgaaaaatgggggcccgaacttgatattgaaaaaaccaaattgctttatacagcaggccagtcaacacatgcaatggaaatgttgagttcttgtgccatatctttctgcaagtctgtgaaagctgaatatgcagttgctaaatcaattctgacactggctaaatggatccaggcagaatggaaagagatttcaggacagctgaaacaggtttacagagctcagcaccaacagaacttcacaggtctttctactttgtctaaaaacatactcactctaatagaactgccatctgttaatacgatggaagaagagtatcctcggatcgagagtgaatctacagtgcatattggagttggagaacctgacttcattttgggacagttgtatcacctgtcttcagtacaggcacctgaagtagccaaatcttgggcagcgttggccagctgggcttataggtggggcagaaaggtggttgacaatgccagtcagggagaaggtgttcgtctgctgcctagagaaaaatctgaagttcagaatctacttccagacactataactgaggaagagaaagagagaatatatggtattcttggacaggctgtgtgtcggccggcggggattcaggatgaagatataacacttcagataactgagagtgaagacaacgaagaagatgacatggttgatgttatctggcgtcagttgatatcaagctgcccatggctttcagaacttgatgaaagtgcaactgaaggagttattaaagtgtggaggaaagttgtagatagaatattcagcctgtacaaactctcttgcagtgcatactttactttccttaaactcaacgctggtcaaattcctttagatgaggatgaccctaggctgcatttaagtcacagagtggaacagagcactgatgacatgattgtgatggccacattgcgcctgctgcggttgctcgtgaagcatgctggtgagcttcggcagtatctggagcacggcttggagacaacacccactgcaccatggaggggaattattccgcaacttttctcacgcttaaaccaccctgaagtgtatgtgcgccaaagtatttgtaaccttctctgccgtgtggctcaagattccccacatctcatattgtatcctgcaatagtgggtaccatatcgcttagtagtgaatcccaggcttcaggaaataaattttccactgcaattccaactttacttggcaatattcaaggagaagaattgctggtttctgaatgtgagggaggaagtcctcctgcatctcaggatagcaataaggatgaacctaaaagtggattaaatgaagaccaagccatgatgcaggattgttacagcaaaattgtagataagctgtcctctgcaaaccccaccatggtattacaggttcagatgctcgtggctgaactgcgcagggtcactgtgctctgggatgagctctggctgggagttttgctgcaacaacacatgtatgtcctgagacgaattcagcagcttgaagatgaggtgaagagagtccagaacaacaacaccttacgcaaagaagagaaaattgcaatcatgagggagaggcacacagctttgatgaagcccatcgtatttgctttggagcatgtgaggagtatcacagcggctcctgcagaaacacctcatgaaaaatggtttcaggataactatggtgatgccattgaaaatgccctagaaaaactgaagactccattgaaccctgcaaagcctgggagcagctggattccatttaaagagataatgctaagtttgcaacagagagcacagaaacgtgcaagttacatcttgcgtcttgaagaaatcagtccatggttggctgccatgactaacactgaaattgctcttcctggggaagtctcagccagagacactgtcacaatccatagtgtgggcggaaccatcacaatcttaccgactaaaaccaagccaaagaaacttctctttcttggatcagatgggaagagctatccttatcttttcaaaggactggaggatttacatctggatgagagaataatgcagttcctatctattgtgaataccatgtttgctacaattaatcgccaagaaacaccccggttccatgctcgacactattctgtaacaccactaggaacaagatcaggactaatccagtgggtagatggagccacacccttatttggtctttacaaacgatggcaacaacgggaagctgccttacaagcacaaaaggcccaagattcctaccaaactcctcagaatcctggaattgtaccccgtcctagtgaactttattacagtaaaattggccctgctttgaaaacagttgggcttagcctggatgtgtcccgtcgggattggcctcttcatgtaatgaaggcagtattggaagagttaatggaggccacacccccgaatctccttgccaaagagctctggtcatcttgcacaacacctgatgaatggtggagagttacgcagtcttatgcaagatctactgcagtcatgtctatggttggatacataattggccttggagacagacatctggataatgttcttatagatatgacgactggagaagttgttcacatagattacaatgtttgctttgaaaaaggtaaaagccttagagttcctgagaaagtaccttttcgaatgacacaaaacattgaaacagcactgggtgtaactggagtagaaggtgtatttaggctttcatgtgagcaggttttacacattatgcggcgtggcagagagaccctgctgacgctgctggaggcctttgtgtacgaccctctggtggactggacagcaggaggcgaggctgggtttgctggtgctgtctatggtggaggtggccagcaggccgagagcaagcagagcaagagagagatggagcgagagatcacccgcagcctgttttcttctagagtagctgagattaaggtgaactggtttaagaatagagatgagatgctggttgtgcttcccaagttggacggtagcttagatgaatacctaagcttgcaagagcaactgacagatgtggaaaaactgcagggcaaactactggaggaaatagagtttctagaaggagctgaaggggtggatcatccttctcatactctgcaacacaggtattctgagcacacccaactacagactcagcaaagagctgttcaggaagcaatccaggtgaagctgaatgaatttgaacaatggataacacattatcaggctgcattcaataatttagaagcaacacagcttgcaagcttgcttcaagagataagcacacaaatggaccttggtcctccaagttacgtgccagcaacagcctttctgcagaatgctggtcaggcccacttgattagccagtgcgagcagctggagggggaggttggtgctctcctgcagcagaggcgctccgtgctccgtggctgtctggagcaactgcatcactatgcaaccgtggccctgcagtatccgaaggccatatttcagaaacatcgaattgaacagtggaagacctggatggaagagctcatctgtaacaccacagtagagcgttgtcaagagctctataggaaatatgaaatgcaatatgctccccagccacccccaacagtgtgtcagttcatcactgccactgaaatgaccctgcagcgatacgcagcagacatcaacagcagacttattagacaagtggaacgcttgaaacaggaagctgtcactgtgccagtttgtgaagatcagttgaaagaaattgaacgttgcattaaagttttccttcatgagaatggagaagaaggatctttgagtctagcaagtgttattatttctgccctttgtacccttacaaggcgtaacctgatgatggaaggtgcagcgtcaagtgctggagaacagctggttgatctgacttctcgggatggagcctggttcttggaggaactctgcagtatgagcggaaacgtcacctgcttggttcagttactgaagcagtgccacctggtgccacaggacttagatatcccgaaccccatggaagcgtctgagacagttcacttagccaatggagtgtatacctcacttcaggaattgaattcgaatttccggcaaatcatatttccagaagcacttcgatgtttaatgaaaggggaatacacgttagaaagtatgctgcatgaactggacggtcttattgagcagaccaccgatggcgttcccctgcagactctagtggaatctcttcaggcctacttaagaaacgcagctatgggactggaagaagaaacacatgctcattacatcgatgttgccagactactacatgctcagtacggtgaattaatccaaccgagaaatggttcagttgatgaaacacccaaaatgtcagctggccagatgcttttggtagcattcgatggcatgtttgctcaagttgaaactgctttcagcttattagttgaaaagttgaacaagatggaaattcccatagcttggcgaaagattgacatcataagggaagccaggagtactcaagttaatttttttgatgatgataatcaccggcaggtgctagaagagattttctttctaaaaagactacagactattaaggagttcttcaggctctgtggtaccttttctaaaacattgtcaggatcaagttcacttgaagatcagaatactgtgaatgggcctgtacagattgtcaatgtgaaaaccctttttagaaactcttgtttcagtgaagaccaaatggccaaacctatcaaggcattcacagctgactttgtgaggcagctcttgatagggctacccaaccaagccctcggactcacactgtgcagttttatcagtgctctgggtgtagacatcattgctcaagtagaggcaaaggactttggtgccgaaagcaaagtttctgttgatgatctctgtaagaaagcggtggaacataacatccagatagggaagttctctcagctggttatgaacagggcaactgtgttagcaagttcttacgacactgcctggaagaagcatgacttggtgcgaaggctagaaaccagtatttcttcttgtaagacaagcctgcagcgggttcagctgcatattgccatgtttcagtggcaacatgaagatctacttatcaatagaccacaagccatgtcagtcacacctcccccacggtctgctatcctaaccagcatgaaaaagaagctgcataccctgagccagattgaaacttctattgcgacagttcaggagaagctagctgcacttgaatcaagtattgaacagcgactcaagtgggcaggtggtgccaaccctgcattggcccctgtactacaagattttgaagcaacgatagctgaaagaagaaatcttgtccttaaagagagccaaagagcaagtcaggtcacatttctctgcagcaatatcattcattttgaaagtttacgaacaagaactgcagaagccttaaacctggatgcggcgttatttgaactaatcaagcgatgtcagcagatgtgttcgtttgcatcacagtttaacagttcagtgtctgagttagagcttcgtttattacagagagtggacactggtcttgaacatcctattggcagctctgaatggcttttgtcagcacacaaacagttgacccaggatatgtctactcagagggcaattcagacagagaaagagcagcagatagaaacggtctgtgaaacaattcagaatctggttgataatataaagactgtgctcactggtcataaccgacagcttggagatgtcaaacatctcttgaaagctatggctaaggatgaagaagctgctctggcagatggtgaagatgttccctatgagaacagtgttaggcagtttttgggtgaatataaatcatggcaagacaacattcaaacagttctatttacattagtccaggctatgggtcaggttcgaagtcaagaacacgttgaaatgctccaggaaatcactcccaccttgaaagaactgaaaacacaaagtcagagtatctataataatttagtgagttttgcatcacccttagtcaccgatgcaacaaatgaatgttcgagtccaacgtcatctgctacttatcagccatccttcgctgcagcagtccggagtaacactggccagaagactcagcctgatgtcatgtcacagaatgctagaaagctgatccagaaaaatcttgctacatcagctgatactccaccaagcaccgttccaggaactggcaagagtgttgcttgtagtcctaaaaaggcagtcagagaccctaaaactgggaaagcggtgcaagagagaaactcctatgcagtgagtgtgtggaagagagtgaaagccaagttagagggccgagatgttgatccgaataggaggatgtcagttgctgaacaggttgactatgtcattaaggaagcaactaatctagataacttggctcagctgtatgaaggttggacagcctgggtgtgaatggcaagacagtagatgagtctggttaagcgaggtcagacatccaccagaatcaactcagcctcaggcatccaaagccacaccacagtcggtggtgatgcaactgggggcttactctgaggaaacctaggaaatctcggtgcactaggaagtgaatcccgcaggacagctgcactcagggatacgcccaacaccatggcctgcaaccccagggtcaagggtgaaggaaagcaaagctcaccgcctgaacacggagattgtctttctgccacagaacagcagcagacgtgtcgggaggttagctgcggaaagaaatcgggatgccgcggagcacagagtgatttggaactccattccacctgaccctgtgtgtacaatccaggaaaaaaacaaaccccactcagaaacagagaaaactggggtcgcgaagaaatcacagccaaggaagatttgatgcattcagattctcgtgtaacacttgttgcttggcaacagtactggttgggttgaccagtaagtagaaaaaggctaaaggctatgcgatatgaatttcagaaatggactgaaaatggagagctatgtaacagatacactacagtagaagaacttacttctgaaatgaagggaaaaaaaccaccccatcgttccctactcctccccaccacttacccgttccccctttacctaatctagtagattagccatctttcaaattcacttttatttcagtccttatatttcatatacttccgtctcgatgctgttaacaacttctgataacatggaaaattcaaggattgtttaaaggtctgatgatcacacacaaaatgtaattccggttatttaagtcatttctgtgattctatcatgtacagtttccagaattgtcactgtgcattcaaaagtaatgaatctaacagacatttgatttaatgtacactcccttttgcttatagtgtgcattttttttggaggtcattcaaattttccctcttctgtgatagctgtagtttctttcatagaaagtagctaatccagtgtaatcttttacctttttaaaaaccaagatagagtatctattagagttttacattgttgatgatagattaacaataaagtgatgttctggtggaggtagactgaaatttttttaattcatgtttttcatttgatacttttaatttacacttagtaaattaaaagttgtttaatttacttggcattttaggacatgtacatgaaacagtgaaaatgagatccaccaacatcttttattaagttcagttattagtctgtgaagtgctttactttttgcacaattttaatagcttgctattcagtaatacattatagtgaattcatgatcaaggtttccttaaatttagcattgcatttcagtactgactgtgtaagctaaattgctgatccaaaataaaaacccagactagaatagggttcttaaaatcaagtatcaatacaaaatagaacacaattaaaatcttaattgttggctgggcacagtggctcacgcctgtaatcccagcactttgggaggccgaggcgggcggatcatgaggttaggagagcgagaccatcctggctaacacggtgaaaccccgtctttactaaaatacaaaaaaaattagccgggtgtggtggcgggcgcctgtagtcccagctactcgggaggctgaggcaggagaatggcgtgaacccaggaggcggagcttgcagtgagccgagattgtgccactgcactccagcctgggcaacagagctagactctgtgtcaaaaataaatgactagat

SEQ ID NO: 10 - protein, 3657 aa, Homo sapiens [sequence omitted]

SEQ ID NO: 11 - DNA, 20 bp, Artificial Sequence
  synthetic oligonucleotide
agcaagctccctcctgtctc